SEQUENCE LISTING (63 sequences)

SEQ ID NO: 1 - DNA, 1497 bp, Mus musculus
ctgcagcttgttctttaatgtcaggagactctcccttctgcttgtcctggtgggccctggggggagcggggagggaatacctaagagcaattggtagctggtacttctaatgcctcttcctcctccaacctccaagagtctgttttgggattgggttcaggaatgaaattctgcctgtgctaacctcctggggagccggtagacttgtctgttaaaaatcgcttctgcttttggagcctaaagcccggttccgaaaaacaagtggtatttaggggaaagaggggtcttcaaaggctacagtgagtcattccagccttcaaccatactacgccagcactacgttctctaaagccactctgcgctagcttgcggtgaggggaggggagaaaaggaaaggggaggggaggggaggggagggagaaaggaggtgggaaggcagagaggccggctgcgggggcgggaccgactcacaaactgttcgatttcgtttccacctcccagcgccccctcggagatccctaggagccagcctgctgggagaaccagagggtccggagcaaacctggaggctgagagggcatcagaggggaaaagactgagctagccactccagtgccatacagaagcttaagggacgcaccacgccagccccagcccagcgacagccaacgcctgttgcagagcggcggcttcgaagccgccgcccaggagctgccctttcctcttcggtgaagtttctaaaagctgcgggagactcagaggaagcaaggaaagtgtccggtaggactacggctgcctttgtcctcttcccctctacccttaccccctcctgggtcccctctccaggagctgactaggcaggctttctggccaaccctctcccctacacccccagctctgccagccagtttgcacagaggtaaactccctttggctgagagtaggggagcttgttgcacattgcaaggaaggcttttgggagcccagagactgaggagcaacagcacgcccaggagagtccctggttccaggttctcgcccctgcacctcctcctgcccgcccctcaccctgtgtgtggtgttagaaatgaaaagatgaaaaggcagctagggtttcagtagtcgaaagcaaaacaaaagctaaaagaaaacaaaaagaaaatagcccagttcttatttgcacctgcttcagtggactttgaatttggaaggcagaggatttccccttttccctcccgtcaaggtttgagcatcttttaatctgttcttcaagtatttagagacaaactgtgtaagtagcagggcagatcctgtcttgcgcgtgccttcctttactggagactttgaggttatctgggcactccccccacccaccccccctcctgcaagttttcttccccggagcttcccgcaggtgggcagctagctgcagatactacatcatcagtcaggagaactcttcagagcaagagacgaggaggcaggataagggaattc

SEQ ID NO: 2 - DNA, 600 bp, Mus musculus
ctgcagcttgttctttaatgtcaggagactctcccttctgcttgtcctggtgggccctggggggagcggggagggaatacctaagagcaattggtagctggtacttctaatgcctcttcctcctccaacctccaagagtctgttttgggattgggttcaggaatgaaattctgcctgtgctaacctcctggggagccggtagacttgtctgttaaaaatcgcttctgcttttggagcctaaagcccggttccgaaaaacaagtggtatttaggggaaagaggggtcttcaaaggctacagtgagtcattccagccttcaaccatactacgccagcactacgttctctaaagccactctgcgctagcttgcggtgaggggaggggagaaaaggaaaggggaggggaggggaggggagggagaaaggaggtgggaaggcagagaggccggctgcgggggcgggaccgactcacaaactgttcgatttcgtttccacctcccagcgccccctcggagatccctaggagccagcctgctgggagaaccagagggtccggagcaaacctggaggctgagagggcatcagaggggaaaagactgag

SEQ ID NO: 3 - DNA, 359 bp, Mus musculus
cccaagcgctagtgttctgttctctttttgtaatcttggaatcttttgttgctctaaatacaattaaaaatggcagaaacttgtttgttggaatacatgtgtgactcttggtttgtctctgcgtctggctttagaaatgtcatccattgtgtaaaatactggcttgttggtctgccagctaaaacttgccacagcccctgttgtgactgcaggctcaagttattgttaacaaagagccccaagaaaagctgctaatgtcctcttatcaccattgttaatttgttaaaacataaaacaatctaaaatttcagatgaatgtcatcagagttcttttcattagctctttttattggctgtct

SEQ ID NO: 4 - protein, 899 aa, Mus musculus
MEVQLGLGRVYPRPPSKTYRGAFQNLFQSVREAIQNPGPRHPEAANIAPPGACLQQRQETSPRRRRRQQHTEDGSPQAHIRGPTGYLALEEEQQPSQQQAASEGHPESSCLPEPGAATAPGKGLPQQPPAPPDQDDSAAPSTLSLLGPTFPGLSSCSADIKDILNEAGTMQLLQQQQQQQQHQQQHQQHQQQQEVISEGSSARAREATGAPSSSKDSYLGGNSTISDSAKELCKAVSVSMGLGVEALEHLSPGEQLRGDCMYASLLGGPPAVRPTPCAPLPECKGLPLDEGPGKSTEETAEYSSFKGGYAKGLEGESLGCSGSSEAGSSGTLEIPSSLSLYKSGALDEAAAYQNRDYYNFPLALSGPPHPPPPTHPHARIKLENPLDYGSAWAAAAAQCRYGDLGSLHGGSVAGPSTGSPPATTSSSWHTLFTAEEGQLYGPGGGGGSSSPSDAGPVAPYGYTRPPQGLTSQESDYSASEVWYPGGVVNRVPYPSPNCVKSEMGPWMENYSGPYGDMRLDSTRDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGENSNAGSPTEDPSQKMTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 5 - DNA, 2988 bp, Mus musculus
gcttcccgcaggtgggcagctagctgcagatactacatcatcagtcaggagaactcttcagagcaagagacgaggaggcaggataagggaattcggtggaagctacagacaagctcaaggatggaggtgcagttagggctgggaagggtctacccacggcccccatccaagacctatcgaggagcgttccagaatctgttccagagcgtgcgcgaagcgatccagaacccgggccccaggcaccctgaggccgctaacatagcacctcccggcgcctgtttacagcagaggcaggagactagcccccggcggcggcggcggcagcagcacactgaggatggttctcctcaagcccacatcagaggccccacaggctacctggccctggaggaggaacagcagccttcacagcagcaggcagcctccgagggccaccctgagagcagctgcctccccgagcctggggcggccaccgctcctggcaaggggctgccgcagcagccaccagctcctccagatcaggatgactcagctgccccatccacgttgtccctgctgggccccactttcccaggcttaagcagctgctccgccgacattaaagacattttgaacgaggccggcaccatgcaacttcttcagcagcagcaacaacagcagcagcaccaacagcagcaccaacagcaccaacagcagcaggaggtaatctccgaaggcagcagcgcaagagccagggaggccacgggggctccctcttcctccaaggatagttacctagggggcaattcaaccatatctgacagtgccaaggagttgtgtaaagcagtgtctgtgtccatgggattgggtgtggaagcattggaacatctgagtccaggggaacagcttcggggagactgcatgtacgcgtcgctcctgggaggtccacccgcggtgcgtcccactccttgtgcgccgctgcccgaatgcaaaggtcttcccctggacgaaggcccaggcaaaagcactgaagagactgctgagtattcctctttcaagggaggttacgccaaaggattggaaggtgagagcttggggtgctctggcagcagtgaagcaggtagctctgggacacttgagatcccgtcctctctgtctctgtataaatctggagcactagacgaggcagcagcataccagaatcgcgactactacaactttccgctggctctgtccgggccgccgcaccccccgccccctacccatccacacgcccgtatcaagctggagaacccattggactacggcagcgcctgggctgcggcggcagcgcaatgccgctatggggacttgggtagtctacatggagggagtgtagccgggcccagcactggatcgcccccagccaccacctcttcttcctggcatactctcttcacagctgaagaaggccaattatatgggccaggaggcgggggcggcagcagcagcccaagcgatgccgggcctgtagccccctatggctacactcggccccctcaggggctgacaagccaggagagtgactactctgcctccgaagtgtggtatcctggtggagttgtgaacagagtaccctatcccagtcccaattgtgtcaaaagtgaaatgggaccttggatggagaactactccggaccttatggggacatgcgtttggacagtaccagggaccatgttttacccatcgactattactttccaccccagaagacctgcctgatctgtggagatgaagcttctggctgtcactacggagctctcacttgtggcagctgcaaggtcttcttcaaaagagccgctgaagggaaacagaagtatctatgtgccagcagaaacgattgtaccattgataaatttcggaggaaaaattgcccatcttgtcgtctccggaaatgttatgaagcagggatgactctgggagctcgtaagctgaagaaacttggaaatctaaaactacaggaggaaggagaaaactccaatgctggcagccccactgaggacccatcccagaagatgactgtatcacacattgaaggctatgaatgtcagcctatctttcttaacgtcctggaagccattgagccaggagtggtgtgtgccggacatgacaacaaccaaccagattcctttgctgccttgttatctagcctcaatgagcttggagagaggcagcttgtgcatgtggtcaagtgggccaaggccttgcctggcttccgcaacttgcatgtggatgaccagatggcggtcattcagtattcctggatgggactgatggtatttgccatgggttggcggtccttcactaatgtcaactccaggatgctctactttgcacctgacttggttttcaatgagtaccgcatgcacaagtctcggatgtacagccagtgtgtgaggatgaggcacctgtctcaagagtttggatggctccaaataaccccccaggaattcctgtgcatgaaagcactgctgctcttcagcattattccagtggatgggctgaaaaatcaaaaattctttgatgaacttcgaatgaactacatcaaggaactcgatcgcatcattgcatgcaaaagaaagaatcccacatcctgctcaaggcgcttctaccagctcaccaagctcctggattctgtgcagcctattgcaagagagctgcatcagttcacttttgacctgctaatcaagtcccatatggtgagcgtggactttcctgaaatgatggcagagatcatctctgtgcaagtgcccaagatcctttctgggaaagtcaagcccatctatttccacacacagtgaagatttggaaaccctaatacccaaaacccaccttgttccctttccagatgtcttctgcctgttatataactctgcactacttctctgcagtgccttgggggaaattcctctactgatgtacagtcagacgtgaacaggttcctcagttctatttcctgggcttctcct

SEQ ID NO: 6 - protein, 899 aa, Mus musculus; identical to SEQ ID NO: 4

SEQ ID NO: 7 - DNA, 2988 bp, Mus musculus; identical to SEQ ID NO: 5

SEQ ID NO: 8 - protein, 899 aa, Mus musculus; identical to SEQ ID NO: 4

SEQ ID NO: 9 - DNA, 2700 bp, Mus musculus
atggaggtgcagttagggctgggaagggtctacccacggcccccatccaagacctatcgaggagcgttccagaatctgttccagagcgtgcgcgaagcgatccagaacccgggccccaggcaccctgaggccgctaacatagcacctcccggcgcctgtttacagcagaggcaggagactagcccccggcggcggcggcggcagcagcacactgaggatggttctcctcaagcccacatcagaggccccacaggctacctggccctggaggaggaacagcagccttcacagcagcaggcagcctccgagggccaccctgagagcagctgcctccccgagcctggggcggccaccgctcctggcaaggggctgccgcagcagccaccagctcctccagatcaggatgactcagctgccccatccacgttgtccctgctgggccccactttcccaggcttaagcagctgctccgccgacattaaagacattttgaacgaggccggcaccatgcaacttcttcagcagcagcaacaacagcagcagcaccaacagcagcaccaacagcaccaacagcagcaggaggtaatctccgaaggcagcagcgcaagagccagggaggccacgggggctccctcttcctccaaggatagttacctagggggcaattcaaccatatctgacagtgccaaggagttgtgtaaagcagtgtctgtgtccatgggattgggtgtggaagcattggaacatctgagtccaggggaacagcttcggggagactgcatgtacgcgtcgctcctgggaggtccacccgcggtgcgtcccactccttgtgcgccgctgcccgaatgcaaaggtcttcccctggacgaaggcccaggcaaaagcactgaagagactgctgagtattcctctttcaagggaggttacgccaaaggattggaaggtgagagcttggggtgctctggcagcagtgaagcaggtagctctgggacacttgagatcccgtcctctctgtctctgtataaatctggagcactagacgaggcagcagcataccagaatcgcgactactacaactttccgctggctctgtccgggccgccgcaccccccgccccctacccatccacacgcccgtatcaagctggagaacccattggactacggcagcgcctgggctgcggcggcagcgcaatgccgctatggggacttgggtagtctacatggagggagtgtagccgggcccagcactggatcgcccccagccaccacctcttcttcctggcatactctcttcacagctgaagaaggccaattatatgggccaggaggcgggggcggcagcagcagcccaagcgatgccgggcctgtagccccctatggctacactcggccccctcaggggctgacaagccaggagagtgactactctgcctccgaagtgtggtaccctggtggagttgtgaacagagtaccctatcccagtcccaattgtgtcaaaagtgaaatgggaccttggatggagaactactccggaccttatggggacatgcgtttggacagtaccagggaccatgttttacccatcgactattactttccaccccagaagacctgcctgatctgtggagatgaagcttctggctgtcactacggagctctcacttgtggcagctgcaaggtcttcttcaaaagagccgctgaagggaaacagaagtatctatgtgccagcagaaacgattgtaccattgataaatttcggaggaaaaattgcccatcttgtcgtctccggaaatgttatgaagcagggatgactctgggagctcgtaagctgaagaaacttggaaatctaaaactacaggaggaaggagaaaactccaatgctggcagccccactgaggacccatcccagaagatgactgtatcacacattgaaggctatgaatgtcagcctatctttcttaacgtcctggaagccattgagccaggagtggtgtgtgccggacatgacaacaaccaaccagattcctttgctgccttgttatctagcctcaatgagcttggagagaggcagcttgtgcatgtggtcaagtgggccaaggccttgcctggcttccgcaacttgcatgtggatgaccagatggcggtcattcagtattcctggatgggactgatggtatttgccatgggttggcggtccttcactaatgtcaactccaggatgctctactttgcacctgacttggttttcaatgagtaccgcatgcacaagtctcggatgtacagccagtgtgtgaggatgaggcacctgtctcaagagtttggatggctccaaataaccccccaggaattcctgtgcatgaaagcactgctgctcttcagcattattccagtggatgggctgaaaaatcaaaaattctttgatgaacttcgaatgaactacatcaaggaactcgatcgcatcattgcatgcaaaagaaagaatcccacatcctgctcaaggcgcttctaccagctcaccaagctcctggattctgtgcagcctattgcaagagagctgcatcagttcacttttgacctgctaatcaagtcccatatggtgagcgtggactttcctgaaatgatggcagagatcatctctgtgcaagtgcccaagatcctttctgggaaagtcaagcccatctatttccacacacagtga

SEQ ID NO: 10 - DNA, 2217 bp, Mus musculus
  misc_feature 626, 1791, 1795, 2193: n = A,T,C or G
cacccccccaatcccctacccacccactccccctttttggccctggcgttcccctgtactggggcatataaagtttgcaagtccaatgggcctctctctttgccatgatggccgactaggccatcttttgatacatatgcagctaaagacaagagctcccgggtactggttagttcatattgttgttccacctatagggttgcagttccctttagctccttgggtaatttctctagctcctccattaggggccgtgtgacccatccaatagctgactgtgatcatccacttctgtgtttgctaggccccgacatagtctcacaagagagagctataactgggtcctttcagcgaaatcttgctagtgtatgcaatggtgtcagcatttggaagctgattatgggatggatccctgcatatggcatctattacatttttgttacagaacagggaaagggacactgagagactcaagaagaaagaaaaggaattaatacaaaagaacagtgaaagctggtatgataatactaatttatcctttacttgtatattaatatcaagagtaactcatacatctgatttatgttgtcagagcaataactcagtactactggtagcaatattgntgtttttacagggtaagactctaggctccaagagctaaaatatataaaattcttctggtatttgataaggctgatcataggcctctctctggaagaagtaagatagagttatgttcatgccatttaatgactgtatatgtcgtcattaatgcatcacattaagttgataccttaacctctgcttaacttccttctcttacaaatgcagagctcatgagattggctattccctcagaacctgtttaattccttggcaggattcaaagtgtccataggaaaccttacaaacactctgtccagagaaggtctcaaaagagttcagctttacactgattcactcgagcaatccatagaatagtcacttggatgtatgtacagtttctcagaagaccgtagaattctgatcgatgtctgccatccactgacatatgttgctttgttctctctctgtctctgtgtgtgtcttttcagtttggacagtaccagggaccatgttttacccatcgactattactttccaccccagaagacctgcctgatctgtggagatgaagcttctggctgtcactacggagctctcacttgtggcagctgcaaggtcttcttcaaaagagccgctgaaggtaaaaagtcttacctacttcctgatattttccccttctcttttgcctagcagagaatgacagtgaccttccagggcattctgataatcccagagactgagtcattagcaagggccctctcacagtacatgtaagatcaaagaagcccatggttatatttgctgagctgtcttggctgccctggttgtacaagcaatgatggtgatgtaggtggtcccagctggtgcttggtggctcccaggactggaagcaaaattaatgatttgaaaaattaaatttccttcctgcttgttttcaactctgcttcctagtgaggaaaagaaaacttgtccttattagagaggttagaagtggagaaaccccaactgagtatacaggctgttttctgtagagaatatgagactgttccttagcaaaagcttcctggctttaaccccagaaaaggaagtgttctcactgttcagcagaccatcagtgtctgcacctgctccctcctgcttgctgcctctttgggacctctctttgcaataagggactccaangcangaaaaaaactcagagagaagcatcagaggactgctttcagggcatgacagttggttcaagaatcccaacgtaacttgcattttgtatccagctaagtgggatggagcctttacttgttatctgcactaattatgatgtttctaacctacatcatctagcagaaacacccactccaggcctttactgtagtcttagtgatccctcccttcttaatcacagggtgggggtgggagcttaaacctttattcatacactctactaccatccctcagtctggtactcctttctcaaagagtcactggaaagctgcccctacatggtctactgtggctgcagactcagttttaaagattcctttgcaactctgccctggtctctggcttcccaccaagggggancttccggccagggaggttttcctt

SEQ ID NO: 11 - DNA, 17 bp, Artificial Sequence
  synthetic construct of primer
cggaattcgtggaagct

SEQ ID NO: 12 - DNA, 22 bp, Artificial Sequence
  synthetic construct of primer
gaaagatctacatcagtagagg

SEQ ID NO: 13 - DNA, 66 bp, Artificial Sequence
  synthetic construct of primer
gtcgggccctatcccagtcccacttgctcgagcaagtgggactgggatagggctttttgaattcgc

SEQ ID NO: 14 - DNA, 25 bp, Artificial Sequence
  synthetic construct of primer
atcatccttctgctcacactgcatg

SEQ ID NO: 15 - DNA, 25 bp, Artificial Sequence
  synthetic construct of primer
acagttgtccgtgtccatgatacgc

SEQ ID NO: 16 - DNA, 23 bp, Artificial Sequence
  synthetic construct of primer
cctgtaaggagtcctgctttgtc

SEQ ID NO: 17 - DNA, 22 bp, Artificial Sequence
  synthetic construct of primer
atgctggctctgccttctgagt

SEQ ID NO: 18 - DNA, 30 bp, Artificial Sequence
  synthetic construct of primer
agacacgcactgaacccgagtctgatgcac

SEQ ID NO: 19 - DNA, 31 bp, Artificial Sequence
  synthetic construct of primer
agacagtacaggtaggggtagtagggatagc

SEQ ID NO: 20 - DNA, 40 bp, Artificial Sequence
  synthetically constructed primer
gtcgggccctatcccagtcccacttgctcgagcaagtggg

SEQ ID NO: 21 - DNA, 40 bp, Artificial Sequence
  synthetically constructed primer
gcgaattcaaaaagccctatcccagtcccacttgctcgag

SEQ ID NO: 22 - DNA, 20 bp, Artificial Sequence
  synthetically constructed primer
ggacagtaccagggaccatg

SEQ ID NO: 23 - DNA, 20 bp, Artificial Sequence
  synthetically constructed primer
tccgtagtgacagccagaag

SEQ ID NO: 24 - DNA, 20 bp, Artificial Sequence
  synthetically constructed primer
taatggtggaccgcaacaac

SEQ ID NO: 25 - DNA, 22 bp, Artificial Sequence
  synthetically constructed primer
gtattccgtctccttggttcag

SEQ ID NO: 26 - DNA, 18 bp, Artificial Sequence
  synthetically constructed primer
cagagcggagggtgaatg

SEQ ID NO: 27 - DNA, 19 bp, Artificial Sequence
  synthetically constructed primer
ggcgaaggcagcaattatc

SEQ ID NO: 28 - DNA, 20 bp, Artificial Sequence
  synthetically constructed primer
aggagtggacaatgaagatg

SEQ ID NO: 29 - DNA, 19 bp, Artificial Sequence
  synthetically constructed primer
tgagcagaagttggcatag

SEQ ID NO: 30 - DNA, 18 bp, Artificial Sequence
  synthetically constructed primer
cactatgagcgggttgtc

SEQ ID NO: 31 - DNA, 18 bp, Artificial Sequence
  synthetically constructed primer
ggtgctggtggcgttaga

SEQ ID NO: 32 - DNA, 18 bp, Artificial Sequence
  synthetically constructed primer
ctacacccactccattcc

SEQ ID NO: 33 - DNA, 19 bp, Artificial Sequence
  synthetically constructed primer
gcaggttccgagtaagtaa

SEQ ID NO: 34 - DNA, 18 bp, Artificial Sequence
  synthetically constructed primer
gggctttgaggctgtcta

SEQ ID NO: 35 - DNA, 19 bp, Artificial Sequence
  synthetically constructed primer
aagggtccattcaggtgta

SEQ ID NO: 36 - DNA, 19 bp, Artificial Sequence
  synthetically constructed primer
atctccacccgagttacca

SEQ ID NO: 37 - DNA, 22 bp, Artificial Sequence
  synthetically constructed primer
aacttagccctgtatgtcttca

SEQ ID NO: 38 - DNA, 18 bp, Artificial Sequence
  synthetically constructed primer
aatgctgcgaggagtggg

SEQ ID NO: 39 - DNA, 22 bp, Artificial Sequence
  synthetically constructed primer
ggctctaatcatagttgggtct

SEQ ID NO: 40 - DNA, 20 bp, Artificial Sequence
  synthetically constructed primer
tggtcctgctgctcctcttg

SEQ ID NO: 41 - DNA, 19 bp, Artificial Sequence
  synthetically constructed primer
ccgctgctgctcacacttc

SEQ ID NO: 42 - DNA, 18 bp, Artificial Sequence
  synthetically constructed primer
taccgagacctgaagtgg

SEQ ID NO: 43 - DNA, 21 bp, Artificial Sequence
  synthetically constructed primer
tctgagtcctgtagtagtggg

SEQ ID NO: 44 - DNA, 19 bp, Artificial Sequence
  synthetically constructed primer
agaggtacgctacgaagtc

SEQ ID NO: 45 - DNA, 18 bp, Artificial Sequence
  synthetically constructed primer
gcttgccatcaggattgc

SEQ ID NO: 46 - DNA, 21 bp, Artificial Sequence
  synthetically constructed primer
aggggcactgtcaataccatt

SEQ ID NO: 47 - DNA, 23 bp, Artificial Sequence
  synthetically constructed primer
cgtgaggatactgagaatcccaa

SEQ ID NO: 48 - DNA, 19 bp, Artificial Sequence
  synthetically constructed primer
ggtggatgctcttcagttc

SEQ ID NO: 49 - DNA, 18 bp, Artificial Sequence
  synthetically constructed primer
tttgtaggcttcagtggg

SEQ ID NO: 50 - DNA, 19 bp, Artificial Sequence
  synthetically constructed primer
tatttcaacaagcccacag

SEQ ID NO: 51 - DNA, 20 bp, Artificial Sequence
  synthetically constructed primer
atacatctccagcctcctta

SEQ ID NO: 52 - DNA, 18 bp, Artificial Sequence
  synthetically constructed primer
ggaacaccgacaaaccca

SEQ ID NO: 53 - DNA, 18 bp, Artificial Sequence
  synthetically constructed primer
tccccaaagcacagcaat

SEQ ID NO: 54 - DNA, 18 bp, Artificial Sequence
  synthetically constructed primer
aacggcggcttcttcctg

SEQ ID NO: 55 - DNA, 21 bp, Artificial Sequence
  synthetically constructed primer
tggcacacactcccttgatag

SEQ ID NO: 56 - DNA, 20 bp, Artificial Sequence
  synthetically constructed primer
tcctgccaactctgctctac

SEQ ID NO: 57 - DNA, 22 bp, Artificial Sequence
  synthetically constructed primer
ctttcactttgcctcgtttgtc

SEQ ID NO: 58 - DNA, 19 bp, Artificial Sequence
  synthetically constructed primer
ctgctgttgctgcttcttg

SEQ ID NO: 59 - DNA, 19 bp, Artificial Sequence
  synthetically constructed primer
tgaccttgccgttcttctc

SEQ ID NO: 60 - DNA, 19 bp, Artificial Sequence
  synthetically constructed primer
ctgtgcccttcagattgtt

SEQ ID NO: 61 - DNA, 18 bp, Artificial Sequence
  synthetically constructed primer
ggcggagtgtctttatgc

SEQ ID NO: 62 - DNA, 24 bp, Artificial Sequence
  synthetically constructed primer
tgtgcccatctacgaggggtatgc

SEQ ID NO: 63 - DNA, 21 bp, Artificial Sequence
  synthetically constructed primer
ggtacatggtgccgccagaca

(19) United States
(12) Patent Application Publication
(10) Pub. No.: US 2010/0017900 A1
(43) Pub. Date: Jan. 21, 2010

(54) Prostate Epithelial Androgen Receptor Suppresses Prostate Growth and Tumor Invasion

(75) Inventor: Chawnshang Chang, Pittsford, NY (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: University of Rochester, Rochester, NY (US)

(21) Appl. No.: 12/301,559

(22) PCT Filed: May 21, 2007
(86) PCT No.: PCT/US07/12083

§ 371 Date: Mar. 16, 2009

Related U.S. Application Data
(60) Provisional application No. 60/802,037, filed on May 19, 2006.

(51) Int. Cl. A01K 67/027; A61K 31/7088; A61K 39/395; A61K 31/7105; C12N 15/00; C12Q 1/02; C12N 5/06

(52) U.S. Cl. 800/10; 514/44 R; 424/174.1; 424/134.1; 514/44 A; 435/320.1; 435/29; 435/325; 800/13; 800/18

(57) ABSTRACT

Described herein is the generation of the first conditional knockout AR (pes-ARKO) mice that lack AR only in prostate epithelia. Additionally demonstrated herein, through AR gain- and loss-of-function experiments, are the novel growth and differentiation roles of epithelial AR within the normal and cancerous prostate cells.

### I. BACKGROUND

Androgens and epithelial-mesenchymal interactions are necessary for prostate growth and development. Androgen signaling occurs through the androgen receptor (AR) (Chang, C. S., et al. (1988) Science 240, 324-6; Quigley, C. A. et al. (1995) Endocr Rev 16, 271-321), which is found in both stroma and epithelium within the prostate of all mammals. Mice lacking a functional androgen/AR signaling system fail to develop normal prostate glands (Yeh, S. et al. (2002) Proc Natl Acad Sci USA 99, 13498-503; Wilson, J. D., et al. (1995) Recent Prog Horm Res 50, 349-64). Pioneering developmental studies showed that stroma but not epithelial AR is essential for epithelial cell identity, morphology, bud formation, ductal branching, proliferation, apoptosis, and regulation of secretory profile (Cunha, G. R. & Lung, B. (1978) J Exp Zool 205, 181-93; Cunha, G. R. et al. (2004) J Steroid Biochem Mol Biol 92, 221-36). Experimental evidence has led to the dogmatically held assumption that epithelial AR, when activated by androgen, increases cellular proliferation (Bello, D., et al. (1997) Carcinogenesis 18, 1215-23; Danielpour, D., et al. (1994) Cancer Res 54, 3413-21; Suzuki, H., et al. (2003) Endocr Relat Cancer 10, 209-16). This notion is the central premise for androgen ablation therapy, a key treatment for prostate disease. Although many studies demonstrate that stromal AR mediates key developmental events (Cunha, G. R. & Lung, B. (1978) J Exp Zool 205, 181-93; Cunha, G. R. et al. (2004) J Steroid Biochem Mol Biol 92, 221-36) these studies were typically evaluated over short periods of time, thus recapitulation of events that may take months to manifest a phenotype may not have been sufficiently examined.

In the adult prostate of all mammals, androgen deprivation leads to apoptosis and dedifferentiation of the epithelium, resulting in an increased number of basal cells and decreased population of luminal cells (Evans, G. S. & Chandler, J. (1987) Prostate 11, 339-51; Mirosevich, J. et al. (1999) J Endocrinol 162, 341-50). In benign prostatic hyperplasia and prostate cancer, androgen deprivation decreases growth, increases apoptosis, and reduces tumor volume. Often, however, the effect is temporary, and after removal of androgens, abnormal epithelial cells persist and inevitably grow independent of hormone. Every year more than 30,000 men die and many more suffer from this enigmatic process. To better understand the role of androgen/AR signaling in prostate biology as well as therapeutic targeting, determining the role of epithelial AR is imperative.

### II. SUMMARY

Disclosed are methods and compositions related to epithelial Androgen Receptor (AR).

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the characterization of pes-ARKO mice. FIG. 1a shows wild-type (WT, left) and pes-ARKO mice (KO, right) genotyping. IL-2 RNA occur in both mice and serve as internal positive controls. Transgenes: cre (110 bps) and floxAR (540 bps) are only in pes-ARKO mice. Only WT mice have wild-type AR gene. FIG. 1b shows RT-PCR from priming in exon 1 and exon 3 of the AR gene shows one band for WT AR (305 bps) in WT mice. In pes-ARKO mice (KO), both WT band (WT AR occurs in stroma) and the KO band (153 bps; deletion of exon 2) appears in dorso-lateral prostates (DLP) and ventral prostate (VP), but only the WT band appears in other tissues. FIG. 1c shows the results from external (c) and internal (d) organs looked the same in both strains, except for VP. Note larger size of VP from pes-ARKO mice. FIG. 1e shows the expression of AR (left) and probasin (right) in VPs of WT vs. pes-ARKO mice with age. Probasin levels decline along with epithelial AR. FIG. 1f shows the Pups/litter from WT female×WT males (red bar) or ×pes-ARKO (blue bar) males were similar, left. Serum testosterone levels (f, right) were similar in WT (red bar) and pes-ARKO (blue bar) male mice at week 12 and 24. Key: seminal vesicle (SV), kidney (Kid), ureter (U), anterior prostate (AP), dorsolateral prostate (DLP), ventral prostate (VP) all lobes of prostate (Pr), testes (T), glans penis (Pe); ¤=P<0.05, *=P<0.001.

FIG. 2 shows the histomorphological changes in the ventral prostate of pes-ARKO mice. FIG. 2a shows that the ventral prostate of WT mice show glandular infoldings (arrowheads) and tall secretory epithelium through week 32. In pes-ARKO (KO) littermates, these features were seen only at weeks 3 and 6. In week 9 pes-ARKO mice, some ventral prostate ducts lose glandular infoldings (*) and have short, poorly differentiated epithelial cells compared with WT littermates. The change in epithelia is evident in ˜50% of ducts within the ventral prostate of week 14 pes-ARKO mice. At week 24 (and older), in pes-ARKO mice nearly all glandular infolding and high secretory cells are lost and the enlarged ducts have many sloughed epithelial cells, fragmented nuclei, and immune cells. By week 32 pes-ARKO mice lack glandular infolding and have squat epithelial cells. FIG. 2b-e shows that at week 14, in pes-ARKO mice, the ventral prostate continues to lose glandular infolding. Layers of sloughed epithelial cells are abundant in the prostate lumen. FIG. 2b shows that in week 14 pes-ARKO mice, some glandular infoldings (arrow) appear. FIG. 2c shows that in pes-ARKO mice, infoldings (arrow) become smaller and shorter, and d, infoldings (arrow) lose cellular polarity and constrict (red arrow) at their base. Ultimately, in e, putative glandular infoldings (arrow) are lost and sloughed luminal cells (arrows) appear within the lumen.

FIG. 3 shows the localization of androgen receptor (AR) and probasin (Prb) within ventral prostate of wild-type (WT) vs. pes-ARKO (KO) mice. WT prostate expressed AR in both epithelial and stromal cells (arrows) at all time points evaluated. Cytoplasmic and luminal localization of probasin is first observed at week 3 and decreases from weeks 6-24 in KO vs. WT mice.

FIG. 4 shows that loss of epithelial androgen receptor leads to loss of androgen regulated protein and gene expression and increased proliferation. FIG. 4a shows that Androgen regulated gene transcription decreases as epithelial ARs are lost. Quantitative RT-PCR was done on ventral prostates from WT (red bar) and pes-ARKO (blue bar) mice. Androgen regulated genes probasin, PSP94, and Nkx3.1 are all down-regulated in pes-ARKO prostates compared to WT prostates. FIG. 4b shows that ventral prostates were collected from WT (red bar) and pes-ARKO (blue bar) mice during different stages and analyzed for proliferation. Proliferation, as determined by BrdU positive nuclei primarily occurs in epithelial cells at all stages evaluated and c, epithelial cell proliferation is significantly (P<0.01) higher in pes-ARKO than WT littermates. *=P<0.05.

FIG. 5 shows the localization of epithelial specific markers within the prostate. To determine which epithelial cell populations were increased due to cellular proliferation, immunohistochemistry for basal cell marker p63 a, and luminal cell marker cytokeratin-8 and 18 and epithelial marker pan-cytokeratin b, were performed. Note that an increased number of putative basal cells (arrows; p63 positive) are observed at 32 weeks in pes-ARKO vs. WT mice, whereas, epithelial and luminal cell markers appear to decrease in ventral prostates from pes-ARKO mice vs WT mice.

FIG. 6 shows the sloughing and apoptosis of epithelia in the prostate of pes-ARKO mice. Epithelial cell sloughing is rare in ventral prostates of wild-type (WT) at any time point or in pes-ARKO mice prior to week 16. FIG. 6a shows hematoxylin and eosin staining of pes-ARKO ventral prostates. Note the accumulation of sloughed layers of luminal cells (arrows), cellular debris, putative immune cells, and fragmented nuclei through week 24. FIG. 6b shows the identification of TUNEL-positive cells was performed at weeks 16, 24, and 32. Note that the prostatic cell layer (stroma and epithelium) is not TUNEL positive, whereas TUNEL-positive cells are found within the prostatic lumen. FIG. 6c shows efforts to determine if proliferating (BrdU-positive, green) cells were basal (CK5-positive, red) or luminal (CK8-positive, red) cells we evaluated double staining for BrdU+CK5 vs BrdU+CK8 in pes-ARKO and WT mice. Note that CK8+BrdU-positive cells are rarely seen but BrdU positive cells are found within the basal layer (arrows), whereas CK8+TUNEL positive cells (arrows) are primarily observed within the lumen of pes-ARKO mice with few TUNEL-positive cells observed in the intact epithelial layer. CK5+BrdU positive cells are observed accordingly in the basal layer (arrows) however not all CK5-positive cells are BrdU-positive (note arrowheads). Note that TUNEL-positive cells are CK5-negative (arrows). FIG. 6d shows BrdU-labeling index in different prostatic lobes from pes-ARKO mice at 24 weeks of age.

FIG. 7 shows the expression of T857A mutant androgen receptor transgene in pes-ARKO mice reverts ventral prostate phenotype to wild-type (WT). FIG. 7a shows hemotoxylin and eosin staining of 32 week-old ventral prostates from WT, pes-ARKO, and pes-ARKO/T857A mice. Note that epithelium in pes-ARKO/T857A mice are very similar in morphology, cell height, architecture, and glandular infolding to WT mice. FIG. 7b shows that pes-ARKO/T857A mice have normal AR gene transcription levels and proliferation rates. Quantitative RT-PCR for probasin (orange bars) expression in ventral prostates at 32 weeks. In pes-ARKO/T857A mice probasin expression is significantly increased compared to pes-ARKO mice, but not different compared to WT. Quantitative RT-PCR for PSP-94 (green bars) expression in ventral prostates at week 32. In pes-ARKO/T857A mice PSP-94 expression is significantly increased compared to pes-ARKO mice, but not different compared to WT. BrdU-labeling index (blue bars) in week 32 ventral prostates. In pes-ARKO/T857A mice epithelial cell proliferation is significantly decreased compared to pes-ARKO mice, but not different compared to WT; *=P<0.05, **=P<0.001

FIG. 8 shows Probasin cre and ARKO constructs. FIG. 8a shows the structure of the ARR2PB-Cre-SV40 transgene construct. The plasmid contained the ARR2PB composite PB promoter followed by the cDNA for Cre and SV40 polyadenylation sequence. Arrows indicate the position of the primers used in the PCR-based identification of transgenic mice. FIG. 8b shows the construction of the floxed AR fragment. The PKI vector is modified from the pBluescript plasmid. It contains a T7 promoter at the 3′ end, a T3 promoter at the 5′ end, two multiple cloning sites (MCS), two lox sites, a positive Neo selective marker (PKG-Neo), and a negative thymidine kinase selective marker (MCT-TK). For the cloning, the XhoI site at the 5′ end MCS was first destroyed. A 3-kb intron 2 fragment was introduced into the 3 EcoR1 cloning site (R1), followed by a fragment containing intron 1, exon 2, and a small fragment of intron 2 sequences into 5 XbaI site (X). A lox sequence plus an artificial KpnI site were finally inserted into the XhoI site shortly 5′ to the beginning of exon 2. The constructed plasmid was linearized by NotI before being electroporated into ES cells.

FIG. 9 shows that epithelial AR is a suppressor and stromal AR is a stimulator for the prostate cancer cell invasion in vitro. FIG. 9(a-c) shows knock-in AR in human prostate cancer PC3-v cells results in increased invasion ability. AR protein expression in different PC3-v cell lines transfected with AR cDNA under the control of a natural proximal AR promoter region PC3-AR9 or strong SV40 promoter PC3-AR2 (upper)(a). Transcriptional activity of AR using ARE-(4)-Luc in PC3-AR9 increased 5 fold with 1 nM DHT, addition of 1 μM HF resulted in suppression of DHT-induced transactivation (b). The invasion of PC3-AR9 was decreased in the presence of DHT and was increased with the addition of HF when compared similarly to PC3-v cells grown Matrigel coated Boyden chambers (c). FIG. 9(d-f) shows in vitro tissue recombination assays showed AR played a positive role in PC3-v and PC3-AR9 cell invasion. PC3-v or PC3-AR9 cells cultured on the upper-layer of the Boyden chamber, were co-cultured with WPMY1 vector (WPMY1-v) or WPMY1 AR knockdown (WPMY1-ARsi) cells, which were put in the lower-layer of the chamber as shown (d). WPMY1 cells with Wt AR significantly increase PC3-v or PC3-AR9 cells (upper panel) invasion as compared with WPMY1-ARsi cells (lower panel) (e), and the data were quantitated (f). FIG. 9g shows decreased AR in human prostate cancer CWR22R cells results in increased invasion ability. CWR22R-AR^{+/-} cells in which some alleles of AR gene were genetically disrupted were generated by homologous recombination strategy. Western blot shows that expression of AR is low in the presence or absence of 1 nM DHT in CWR22R-AR^{+/-} cells compared with CWR22R-AR^{+/+} (upper). Transcriptional activity of AR is diminished in CW2R-AR^{+/-} compared with CWR22R-AR^{+/+} in the presence 1 nM DHT (left lower). The invasion into matrigel increased in CWR22R-AR^{+/-} compared with parental CWR2R-AR^{+/+} (right lower). FIG. 9h shows knockdown AR in CWR22R cells using AR-siRNA increased cell invasion in vitro.

FIG. 10 shows the addition of functional AR in PC3-AR9 cells resulted in decreased invasion in in vivo mice models. FIG. 10a shows PC3-AR9 cells formed less osteoclytic lesions than PC3-v cells. Osteoclasts and osteoclast precursors (OC) on cortical bone wafers were cultured with PC3-v and PC3-AR9 cells. After ten days, the wafers were scraped, dried, and stained for tartrate-resistant acid phosphatase (TRAP) for OC cells. The extent of bone resorption with PC3(AR)9 cells decreased compared to PC3-v via measurement by the area of osteoclast lacunae on the bone wafers. OC alone and OC with PTH were used as a negative and positive control respectively. Data are mean±SD *P<0.05, **P<0.01 of three independent experiment and n=3 wild type nude rat (2 day old rat) to isolate the osteoclast precursors. FIG. 10(b, c) shows that PC3-AR9 cells had less ability in bone invasion than that of PC3-v cells. Effects of intra-tibial injection of PC3-v and PC3-AR9 cells in nude mice. PC3-v cells produced larger and more invasive tumors as measured by Dial Caliper in week 12 (b) and higher osteolytic activity in 6-8 weeks radiograph (X-ray) than PC3-AR9 cells (c, arrow). Data are mean±SD * P<0.05, **P<0.01. FIG. 10d shows that PC3-AR9 cells generated smaller metastatic tumors in the lymph nodes than that generated by PC3-v. 5×10⁵ PC3-v and PC3-AR9 cells suspended in 50 μl Matrigel were directly injected into anterior prostate of nude mice. 12 weeks following injection, the tumors were developed and the metastatic tumors in the lymph nodes were compared. FIG. 10e shows that PC3-AR9 cells combined with WPMY1-v and WPMY1-ARsi cells generated smaller metastatic tumors than their control PC3-v combine groups. 5×10 PC3-v or PC3-AR9 cells respectively combined with 5×10⁵ WPMY1-v or WPMY1-ARsi cells were suspended in 50Au Matrigel, and were directly injected into anterior prostate of nude mice. 12 weeks following injection, the tumors were developed and the metastatic tumors in the lymph nodes were compared.

FIG. 11 shows the generation of pes-ARKO-TRAMP Mice. FIG. 11a shows the loss of AR protein expression in prostate epithelium of week 12 pes-ARKO-TRAMP mice compared to WT-TRAMP demonstrated by immunohistochemistry using anti-AR(C19) antibody. FIG. 11b shows similar development of internal urogenital organs of WT-TRAMP and pes-ARKO-TRAMP mice at 6-weeks-old.

FIG. 12 shows that pes-ARKO-TRAMP mice that lack AR only in prostate epithelium develop more aggressive and invasive metastatic tumors. FIG. 12a shows that Pelvic lymph node (PLN) tumors are significantly larger in week 24-pes-ARKO-TRAMP mice compared to WT-TRAMP. FIG. 12b shows the weight of PLN isolated from 24 ws pes-ARKO-TRAMP and Wt TRAMP mice (n=7 mice in each group). FIG. 12c shows the number of liver tumor foci was increased in pes-ARKO-TRAMP mice compared to wt-TRAMP mice (n=4 mice in each group). FIG. 12d shows the expression of AR determined by Western blot analysis of AR protein in PLN tumor, from either week 24 WT-TRAMP or pes-ARKO-TRAMP mice. FIG. 12e shows the higher invasion from pes-ARKO-TRAMP mice PLN tumor primary culture cells compared to those from WT-TRAMP mice using Boyden chamber invasion assay. Addition of functional AR via pBabe virus expressed AR cDNA results in suppression of invasion. The purity/originality of PLN tumor primary culture cells was confirmed by the expression of pan-CK epithelial marker. Data are mean±SD * P<0.05, **P<0.01 of three independent experiment and n=5 mice in each group. FIG. 12f shows that survival was decreased in pes-ARKO-TRAMP (C57BIJ6/129×TRAMP-FVB, n=10) as compared to WT-TRAMP (C57BL/6/129×TRAMP-FVB, n=16).

FIG. 13 shows that ind-ARKO-TRAMP mice delayed in developing metastasis. FIG. 13a shows at 24 wks ind-ARKO-TRAMP mice, which significantly decreased AR expression in both prostate epithelium and stroma, developed smaller tumor with less aggression and metastasis comparing with same-aged Wt tumor. And the metastastic tumor size among groups followed such sequence: pes-ARKO-TRAMP>Wt TRAMP (with and without PIPC)>ind-ARKO-TRAMP. FIG. 13b shows that different tumor malignancy was demonstrated by comparing the metastasis status of similar-sized tumors from groups. In the age of about 22 ws, Wt TRAMP mice developed 1 cm diameter tumors, most of which were well-differentiated tumor with small pelvic lymph node metastasis. In the contrary, pes-ARKO-TRAMP tumor in the similar size developed much larger lymph nodes metastasis in multiple region, even in mesentery, in their earlier age of 18 ws. However, ind-ARKO-TRAMP tumor in 1 cm diameter, although formed as late as 36 ws, invaded into seminal vesicle and migrated to liver. HE staining showed Wt TRAMP tumor were much more well-differentiate than pes-ARKO-TRAMP tumor and ind-ARKO-TRAMP tumor (the second panel). Although AR staining in the pes-ARKO-TRAMP and ind-ARKO-TRAMP tumor was significantly reduced (the third panel), the T-antigen (T-ag) expression in these tumors were not significantly changed (lowest panel).

FIG. 14 shows loss of AR expression in human metastatic tumors compared with primary prostate tumors. FIG. 14a shows the area of high-grade primary tumor showing positive nuclear staining for AR. Immunostain. 400×. FIG. 14b shows the area of high-grade metastatic tumor showing negative staining for AR. Immunostain. 400×. FIG. 14c shows a summary of AR expression comparing primary versus metastatic tumor. None of the tumors displayed neuroendocrine differentiation, and all tissue were obtained from live patients, either as biopsies or lymph node dissections during concurrent prostatectomy. There was no selection for androgen independent metastatic disease. The data for treatment is not available, except in the case for lymph node metastasis, which were all non-treated cases. Metastatic sites included lymph nodes (10), bone (12), liver (1), lung (2), penis/urethra (2), bowel (1). **P<0.01

FIG. 15 shows the influence of AR on the different metastasis/invasion-related genes. FIG. 15a shows that western blot demonstrates a decrease in expression of NEP protein from PLN tumor of pes-ARKO-TRAMP and the PC3-v xenograft compared to those from WT-TRAMP and PC3-AR9 respectively. The transcriptional activity of NEP using NEP-Luc in PC3-AR9 was decreased in the presence of AR-siRNA (si-AR). FIG. 15b shows increased expression of Cox-2 protein in PLN tumor of pes-ARKO-TRAMP and the PC3-v xenograft compare to those from WT-TRAMP and PC3-AR9 tumor xenograft (upper). Transcriptional activity of Cox-2 using Cox-2-Luc in PC3-AR9 was decreased after restoration of functional AR via pBabe virus carrying AR cDNA (lower). FIG. 15c shows decreased p27 protein expression using Western blot from PLN tumor of pes-ARKO-TARMP mice and PC3-v xenografts as compared to those from WT-TRAMP and PC3-AR9 (upper). The increased stability of the cdk inhibitor p27 with AR (middle). PC3-v, and PC3-AR9 cells were treated 48 hr in presence or absence of 1 mM DHT. Cells were then treated with 50 μg/ml cycloheximide (CHX) for indicated times and 50 μg cell lysates were examined by Western blot analysis with an anti-p27 antibody (middle), and the quantitation of the data (middle lower). FIG. 15d shows decreased MMP-9 expression and activity with AR. MMP-9 increased in samples from PLN tumor pes-ARKO-TRAMP and PC3-v tumor xenografts compared to wt-TRAMP and PC3-AR9 tumor xenografts (upper). Enzyme assays of MMP-9 in PC3-AR9 cells further shows that 1 nM DHT can suppress and 1 μM HF can restore the gelatinase activity of MMP-9 (middle upper). Zymography assay for the activity of MMP-9 were increased after treatment with 10 nM TPA (NF-KB activator) and decreased after addition of 1 μg/ml parthenolide (NF-KB inhibitor) (middle lower). Transactivation assay using a NF-kB-Luc in the presence of 1 nM DHT suppresses NF-κB activity in PC-3(AR)9. 1μμM HF restored the effect of DHT (lower). FIG. 15e shows that Akt activity was increased as measured by phosphorylation at serine 473 using p-Akt-473 antibody in Western blot assay of PLN tumor from pes-ARKO-TRAMP and PC3-v xenograft compared to WT-TRAMP and PC3-AR9 (left) and PC3-v and PC3-AR9 derived tumors cells (right). Data are mean±SD * P<0.05, **P<0.01 of three independent experiment and n=3 mice each group. (f) stably knockdown AR expression in human stromal WPMY1 cells significantly changed stroma paracrine factor expressions. Western blots showed AR expression had been knocked down in WPMY1-ARsi cell lines by AR-siRNA (upper panel). Realtime RT-PCR measurement of stroma paracrine factors, which infects tumor metastasis (lower panel), showed the decreased expression of TGFβ1, TGFβ2, TGFβ1, SDF-1 and VEGF.

FIG. 16 shows knock-in AR in prostate cancer cell line PC3 suppressed xenograft tumor growth, and knockdown AR in the prostate stroma cell line WPMY1 also suppressed PC3 xenograft tumor generation in in vivo tissue recombination. FIG. 16a shows that PC3-AR9 cells grew slowly and generated smaller tumors in the anterior prostate than that generated by PC3. 5×10⁵ PC3-v and PC3-AR9 cells suspended in 50 μl Matrigel were directly injected into anterior prostate of nude mice. 12 weeks following injection, the tumors were harvested (upper panel). Ki67, which indicated tumor growth activity, was stained by immunohistochemistry. FIG. 16b shows that PC3 cells combined with WPMY1-v generated smaller tumors than tumors from PC3 and WPMY1-ARsi combination in vivo. 5×10⁵ PC3-v or PC3-AR9 cells respectively combined with 5×10⁵ WPMY1-v or WPMY1-ARsi cells were suspended in 50 μl Matrigel, and were directly injected into anterior prostate of nude mice. 12 weeks following injection, the xenograft tumors were harvested and compared (upper panel). H&E staining show PC3+WPMY1-v cells formed relatively poor differentiated tumor than tumor from PC3-AR9+WPMY1-v in term of lumen formation (middle panel). Ki67 staining showed PC3+WPMY1-v tumor had higher growth rate than PC3-AR9+WPMY1-v tumor (lower panel).

FIG. 17 shows the generation and confirmation of pes-ARKO-TRAMP mice and ind-ARKO-TRAMP mice. FIG. 17a shows the mating strategy of pes-ARKO-TRAMP(C57BL/6/129×TRAMP-FVB) and ind-ARKO-TRAMP mice(C57BL6/129×TRAMP-FVB). FIG. 17b shows the genotype screening of Mice from tail snip DNA. T-ag (SV40) primer was used to identify TRAMP mice at 12 weeks old (upper). Primers 2-3 and select that amplify AR exon2 region were used to identify the Flox/AR in pes-ARKO-TRAMP mice and ind-ARKO-TRAMPmice (middle). Primers specific for Pb-Cre and Mx-Cre were used to identify Pb-Cre and Mx-Cre transgene mice, respectively (lower). FIG. 17c shows that AR knockout were confirmed by detecting the exon2 deletion in AR mRNA. Using exon1 and exon3 primers, specific ARKO bands were shown by RT-PCR amplifying AR mRNA from different organs. In pes-ARKO-TRAMP mice, ARKO bands were shown in Dorsal Lateral Prostate (DLP), Ventral Prostate (VP) and Anterior Prostate (AP), but not significant in Seminal Vesicles (SV) compared to WT-TRAMP. In ind-ARKO-TRAMP mice, ARKO bands were shown in DLP, VP, AP, and SV.

FIG. 18 shows AR expression in pes-ARKO-TRAMP and ind-ARKO-TRAMP mice. FIG. 18a shows the use of Laser Capture Microdissection (LCM) to separate epithelium from stroma, AR exon 2 mRNA expressed in ventral prostate epithelium was amplified by Realtime RT-PCR. pes-ARKO-TRAMP mice lost AR mRNA expression from 6 ws (25%), gradually reached about 50% in 12 ws, and almost disappeared in 16 ws. FIG. 18b shows IHC AR(C-19) staining showed AR protein lost in ventral prostate epithelium (including basal and luminal cells) but not in stroma of 16 ws pes-ARKO-TRAMP mice compared to Wt TRAMP mice. FIG. 18c shows that AR was only knocked out in the epithelium but not in the stroma in ventral prostate of 16 ws pes-ARKO-TRAMP mice using Real-time RT-PCR of AR exon 2 after LCM separating epithelium from stroma. FIG. 18d shows 4 wks and 8 wks following PIPC injection, AR knockout was induced in different organs at various degrees by using Real-time RT-PCR to detect relative expression levels of AR exon 2 mRNA. FIG. 18d shows IHC AR staining showed AR protein partially lost in ventral prostate epithelium and stroma of 16 ws ind-ARKO-TRAMP mice compared to Wt TRAMP mice. FIG. 18f shows that AR was partially knocked out in both epithelium and stroma in ventral prostate of 16 wks ind-ARKO-TRAMP mice using Real-time RT-PCR of AR exon 2 after LCM separating epithelium from stroma.

FIG. 19 shows that ARKO leads to reproductive gross looking changes and cell population changes in prostate tumor. FIG. 19a shows the general gross looking changes of the reproductive organs were observed among 16 wks pes-ARKO-TRAMP, ind-ARKO-TRAMP, castrated TRAMP and Wt TRAMP mice. pes-ARKO-TRAMP mice had enlarged prostates compared with Wt TRAMP mice, with other reproductive organs unchanged. ind-ARKO-TRAMP and castrated TRAMP at 12 ws significantly shrank the size of all reproductive organs, including various lobes of prostates, seminal vesicles, and testis. FIG. 19b shows serum testosterone levels were detected sequentially at 12 ws (before PIPC injection or castration), 16 ws, 20 ws, and 24 ws. The serum T levels remained unchanged in pes-ARKO-TRAMP mice and significantly reduced in ind-ARKO-TRAMP and castrated TRAMP mice. FIG. 19c shows more intermediate cell like population had be observed in pes-ARKO-TRAMP, ind-ARKO-TRAMP, castrated TRAMP and castrated pes-ARKO-TRAMP mice compared with Wt TRAMP mice by double immunofluorescin staining CK5(green) and CK8(red) ventral prostate tumor. FIG. 19d shows that compared with Wt TRAMP tumors, pes-ARKO-TRAMP, ind-ARKO-TRAMP, castrated TRAMP and castrated pes-ARKO-TRAMP tumors expressed higher levels of CD44 cell marker.

FIG. 20 shows the AR negative role in the growth of epithelium tumor was dominated by AR stroma function, which positively stimulates epithelium proliferation through epithelium-stroma interaction. FIG. 20a shows the gross looking and H&E staining of different lobes of the prostates in 16 ws and 20 ws, pes-ARKO-TRAMP mice generated larger tumors than Wt TRAMP mice, while ind-ARKO-TRAMP and castrated TRAMP mice either didn't generate or generate much smaller tumor than their littermate Wt-TRAMP mice. FIG. 20b shows that mice had been sacrificed at different time points of 16 ws, 20 ws and 24 ws, and tumor weight differences had been measured. FIG. 20c shows the tumor growth rates were detected by BrdU incorporation. 24 hs before sacrificed, mice were injected intraperitoneally with BrdU for every 6 hrs. Paraffin fixed tissue sections were stained by special BrdU detecting Kit. FIG. 20d shows double immunofluorescin staining of Ki67(green) and CK5(red) located the proliferation in CK5 positive cells in pes-ARKO-TRAMP mice. Although ind-ARKO-TRAMP and castrated TRAMP also got high percentage of CK5 positive cells, the proliferation in their prostate was still low. FIG. 20e shows that using TUNEL assay, the apoptosis signals in pes-ARKO-TRAMP, ind-ARKO-TRAMP and castration TRAMP were higher than signals from Wt TRAMP. FIG. 20f shows the life span differences among Wt TRAMP, ind-ARKO-TRAMP, pes-ARKO-TRAMP and castrated TRAMP mice were statistically significant.

FIG. 21 shows the mechanism involved in AR suppressor role in the prostatic epithelium. FIG. 21a shows relative expression levels of TGFβ1, TGFβ2, TβR-II, FGF2, FGF7, FGF10, FGF-R1, EGF, EGF-R, SDF1, CXCR4 and VEGF were detected in 16 wks ventral prostate of WT TRAMP and pes-ARKO-TRAMP by Real-time RT-PCR method. FIG. 21b shows TGFβ1, TGFβ2 and TβR-II relative expression levels in 16 wks ventral prostatic epithelium and stroma, separated by laser capture microdissection (LCM), were detected by Real-time RT-PCR method. FIG. 21c shows the relative expression levels of TGFβ1, TGFβ2 and TβR-II in LNCaP cells DHT treated vs. non DHT treated, CWR22R-AR+/+ vs. CWR22R-AR+/− were detected by Real-time RT-PCR method. FIG. 21d shows TGFβ1, TβR-II and phospho-Smad2/3 protein in 16 weeks ventral prostate and 20 weeks prostate tumor were detected by Western blots and IHC staining. Consistent with elevated TGFβ1 and TβR-II protein levels, phospho-Smad2/3 protein was increased in cytoplasm (lower panel) in 16 wks and 20 wks pes-ARKO-TRAMP samples. FIG. 21e shows MAPKs signaling pathways including ERK1/2, JNK, and p38, which TGFβ signaling cross-talked with, had been enhanced in pes-ARKO-TRAMP 16 wks ventral prostates and 20 wks prostate tumors. FIG. 21f shows that in 16 wks ventral prostates and 20 wks prostate tumors of pes-ARKO-TRAMP mice, relatively higher levels of EGF-R, FGF-R1 and CXCR4, were observed, which explains why higher levels of phospho-Akt (shown by both W.B. and IHC) and phospho-CREB. FIG. 21g shows the consequence of over-activated MAPKs signaling and elevated phospho-Akt and phospho-CREB in pes-ARKO-TRAMP prostate results in lower levels of p16 and p21, and higher levels of cyclin D1 comparing with their littermates.

FIG. 22 shows the mechanism involved in AR stimulator role in the prostatic stroma. FIG. 22a shows Western Blots and Real-time RT-PCR showed AR had been knocked down in WPMY1-ARsi cells. FIG. 22b shows Realtime RT-PCR detected the relative expression levels of stromal paracrine factor FGF2, FGF7 and FGF10. FGF2, FGF7 and FGF10 expression were lower in WPMY1-ARsi cells and 16 wks ventral prostate of ind-ARKO-TRAMP mice compared with in WPMY1-v cells and 16 ws Wt TRAMP samples respectively. FIG. 22c shows the relative higher expressed levels of INHBA and BMP4 were observed in 16 wks ventral prostate of ind-ARKO-TRAMP and WPMY1-ARsi cells compared with 16 wks Wt TRAMP samples and WPMY1-v cells. TGFβ1 was also elevated in ind-ARKO-TRAMP mice. FIG. 22d shows the relative lower expression levels of HB-EGF, IGF1 and SDF1 were found in 16 wks ventral prostate of ind-ARKO-TRAMP mice than that of WT TRAMP mice, and had been confirmed as lower expression levels in WPMY1-ARsi cells.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms a, “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Androgen Receptor

Androgen receptor belongs to a superfamily of steroid hormone receptors and was first subcloned in 1988 (Chang, 1988). It contains an N-terminal transactivation domain, a central DNA binding domain (DBD) and a C-terminal ligand binding domain (LBD) (Umesono, 1995). By forming a homodimer and taking into account of the ligand and coregulators, the androgen receptors interact and regulate the transcription of numerous target genes (1 ng, 1992; Schulman, 1995; Beatp, 1996; Yeh, 1996; Glass, 1997, Shibata, 1997). Androgen is the strongest ligand of the androgen receptor. However, it is not the only ligand. Estradiol has been found to activate androgen receptor transactivation through the interaction with androgen receptor (Yeh, 1998). Also, androgen and androgen receptor do not only act in males. The increasing evidence has displayed that the androgen and androgen receptor (AR) may also play important role in female physiological processes, including the process of folliculogenesis, the bone metabolism and the maintenance of brain functions (Miller, 2001).

Androgen is the most conspicuous amount of steroid hormone in the ovaries (Risch H A, 1998). The concentrations of testosterone and estradiol in the late-follicular phase when estrogens are at their peak are 0.06-0.10 mg/day and 0.04-0.08 mg·day respectively (Risch H A, 1998). The ratio of androgens versus estrogens in the ovarian veins of postmenopausal women is 15 to 1 (Risch, 1998; Doldi N, 1998). Androgen receptor is expressed dominantly in granulosa cells of the ovary (Hiller S G, 1992; Hild-Petito S, 1991). With the overproduction of ovarian androgen, women with polycystic ovarian syndrome suffered from impairment of ovulatory function which is characterized with the increasing number of small antral follicles, but arrest in grafian follicles development (Kase, 1963; Futterweit W, 1986; Pache T D, 1991; Spinder T, 1989; Spinder T, 1989; Hughesdon P E, 1982). This symptom has suggested that AR may play a proliferative role in early folliculogenesis but turn to inhibitory effect in late folliculogenesis. The recent studies conducted in animals have supported this hypothesis (Harlow C R, 1988; Hillier S, 1988; Weil S, 1998; Vendola K, 1998; Weil S, 1999; Vendola K, 1999). Administration of dihydroxytestosterone (DHT) in rhesus monkeys has increased the number of primary, preantral and small antral follicles. Since DHT is the metabolite of testosterone and cannot be aromatized, the result suggested the proliferative effect was through AR system (Vendola K, 1999).

### C. Method of Treating Cancer

Disclosed herein is the concept that the Androgen ablation therapy currently used in the art indiscriminately antagonizes stromal AR to prevent proliferation and in doing so ignores the role of epithelial AR in prostate homeostasis. Epithelial AR acts as a suppressor to suppress epithelial proliferation as well as invasiveness and metastatic potential of prostate tumors. Loss of epithelial AR through Androgen ablation therapy results in a loss of AR and thus enhances invasiveness and metastatic potential. Additionally, loss of epithelial AR stimulates mitogenesis. This is in contrast to the established effects of androgen ablation therapy on stromal tissue.

Therefore, disclosed herein are methods of selectively inhibiting cellular proliferation or treating a cancer through targeted androgen or anti-androgen therapy. It is understood and herein contemplated that the disclosed methods can promote AR driven epithelial suppression. Thus, for example, disclosed herein are methods of inhibiting cellular proliferation in a subject comprising administering to the subject AR directed to the epithelial cells. Also disclosed are methods of inhibiting cellular proliferation in a subject comprising administering to the subject androgen directed to the epithelial cells. Also disclosed are methods of treating cancer in a subject comprising administering to the subject androgen directed to the epithelial cells.

It is further understood that one way to prevent the inhibitin of the suppressive effects epithelial AR on cellular proliferation is through the targeted application of anti-androgen therapy directed specifically to the stromal cells. Thus disclosed herein are methods of treating a cancer or inhibiting cellular proliferation in a subject comprising administering to the an anti-androgen or anti-androgen receptor directed to the stromal cells.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Treatment,” “treat,” or “treating” mean a method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression. For example, a disclosed method for reducing the effects of prostate cancer is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition.

A “decrease” can refer to any change that results in a smaller amount of a composition or compound, such as AR. Thus, a “decrease” can refer to a reduction in an activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed.

An “increase” can refer to any change that results in a larger amount of a composition or compound, such as AR relative to a control. Thus, for example, an increase in the amount in AR can include but is not limited to a 10%, 20%, 30%, 40%, 50%, 60%, 70%, 80%, 90%, or 100% increase.

It is understood and herein contemplated that the androgen or androgen receptor can be administered directly or comprised in a vector. It is also understood that the vector can be targeted directly to epithelial cells or the androgen gene or androgen receptor gene encoded on the vector can be operably linked to a tissue specific promoter. Therefore, disclosed herein are vectors comprising androgen or androgen receptor, wherein the androgen or androgen receptor is operably linked to a epithelial tissue specific promoter such as probasin. Thus, it is herein contemplated that the epithelial tissue specific promoter can be specific the prostatic epithelial tissue. It is also contemplated herein that the vector itself can be targeted to a tissue specific site and the androgen gene and/or androgen receptor gene is operably linked to its native promoter. Thus, disclosed herein are methods of inhibiting cellular proliferation or treating cancer in a subject comprising administering to the subject a vector comprising androgen or androgen receptor. Also disclosed are methods of inhibiting cellular proliferation or treating cancer in a subject comprising administering to the subject a vector comprising androgen or androgen receptor, wherein the vector is targeted to the epithelial tissue Also disclosed are methods of inhibiting cellular proliferation or treating cancer in a subject comprising administering to the subject a vector comprising androgen or androgen receptor, wherein the androgen or androgen receptor is operably linked to a tissue specific promoter.

It is understood that androgen promotion of AR driven suppression in epithelial cells can inhibit disregulated cellular proliferation such as cancer. It is also understood that anti-androgen treatment that targets stromal tissue will also inhibit disregulated cellular proliferation such as cancer. Disclosed herein are methods of treating a cancer comprising administering to a subject an anti-androgen agent, wherein the agent inhibits the interaction of androgen and androgen receptor in stromal cells, and wherein the agent does not inhibit the interaction of androgen and androgen receptor in epithelial cells. It is understood that such agent can be any composition that inhibits the interaction of androgen and androgen receptor. Thus, for example, the agent can comprise a siRNA, small molecule, antibody or nonfunctional androgen receptor or androgen. Thus, for example, disclosed herein are agents wherein the agent is an anti-androgen or anti-androgen receptor antibody fusion protein that is targeted to stromal tissue or an anti-androgen or anti-androgen receptor antibody or siRNA that is delivered to the stromal cells via a vector. Thus, disclosed herein are methods of inhibiting cellular proliferation or treating cancer in a subject comprising administering to the subject a vector comprising an anti-androgen or anti-androgen receptor agent, siRNA, or antibody. Also disclosed are methods of inhibiting cellular proliferation or treating cancer in a subject comprising administering to the subject a vector comprising an anti-androgen or anti-androgen receptor, wherein the vector is targeted to the stromal tissue. Also disclosed are methods of inhibiting cellular proliferation or treating cancer in a subject comprising administering to the subject a vector comprising an anti-androgen or anti-androgen receptor, wherein the androgen or androgen receptor is operably linked to a tissue specific promoter.

It is understood and herein contemplated that the disclosed treatment directed to epithelial tissue can be combined with treatments directed to stromal tissue. It is further understood that the treatments can be administered simultaneously or sequentially as the progression of disease dictates. It is understood that those of skill in the art can determine whether to administer androgen therapy to epithelial tissue or anti-androgen therapy to stromal tissue. For example, one of skill in the art can administer an androgen therapy that does not target either epithelial or stromal prostate tissue early in disease progression.

The disclosed compositions can be used to treat any disease where uncontrolled cellular proliferation occurs such as cancers. A non-limiting list of different types of cancers is as follows: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer. It is also understood that the disclosed treatments can be used to treat any known cancer. Thus, for example, it is understood that the disclosed treatments can be used to treat prostate cancer.

Compounds disclosed herein may also be used for the treatment of precancer conditions such as cervical and anal dysplasias, other dysplasias, severe dysplasias, hyperplasias, atypical hyperplasias, and neoplasias.

It is contemplated herein that a tissue-specific agent that modulates androgen androgen receptor interaction or AR—with androgen receptor associated proteins (ARAs) can be used to treat cancer or inhibit disregulated cellular proliferation. Thus, for example, an agent that inhibits the interaction of androgen with AR or AR-ARA in the stroma can be used to treat cancer. Also, for example, an agent that promotes the interaction of androgen with AR or AR-ARA in the epithelia can be used to treat cancer. Disclosed herein are methods of screening for an agent that inhibits prostate growth comprising administering the agent to a prostate cell and monitoring the level of epithelial androgen receptor on the cell, wherein an increase in epithelial androgen receptor relative to a control indicates an agent that inhibits prostate growth. Also disclosed are methods of screening for an agent that inhibits androgen dependent tumor growth comprising administering the agent to a prostate cell and monitoring the level of epithelial androgen receptor on the cell, wherein an increase in epithelial androgen receptor relative to a control indicates an agent that inhibits prostate growth. It is understood that such agents can also be screened for using ex vivo methods. Thus, for example, disclosed herein are methods of screening for an agent that inhibits prostate growth comprising obtaining a tissue sample from a subject, administering the agent to the tissue sample, and monitoring the level of epithelial androgen receptor on the cell, wherein an increase in epithelial androgen receptor relative to a control indicates an agent that inhibits prostate growth.

“Obtaining a tissue sample” or “obtain a tissue sample” means to collect a sample of tissue from a subject or measure a tissue in a subject. It is understood and herein contemplated that tissue samples can be obtained by any means known in the art including invasive and non-invasive techniques. It is also understood that methods of measurement can be direct or indirect. Examples of methods of obtaining or measuring a tissue sample can include but are not limited to tissue biopsy, tissue lavage, aspiration, tissue swab, spinal tap, magnetic resonance imaging (MRI), Computed Tomography (CT) scan, Positron Emission Tomography (PET) scan, and X-ray (with and without contrast media).

### D. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular AR is disclosed and discussed and a number of modifications that can be made to a number of molecules including the AR are discussed, specifically contemplated is each and every combination and permutation of AR and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### 1. Compositions and Methods for Disrupting an AR Loci

The Cre-lox system has been successfully used herein to generate a tissue-specific androgen receptor knockout mice (ARKO). For example, one tissue specific androgen receptor knockout mouse disclosed herein is the prostate epithelial ARKO mouse (pes-ARKO). This principle has been successfully applied for tissue-specific transgene expression (Orban P C, 1992), for site specific gene targeting (Gu, 1994) and for exchange of gene sequence by the “knock-in” method (Hank M, 1995). Disclosed herein, the system has been applied to avoid the infertility problem of male carriers of an androgen receptor knockout and restrict expression of the knockout phenotype to the prostate epithelium. This strategy has been used to generate a knock-out model for prostate cancer progression by crossing the pes-ARKO mouse with a transgenic adenocarcinoma of mouse prostate (TRAMP) mouse to make a pes-ARKO-TRAMP mouse. Disclosed herein is the utilization of the “knock-in” method to generate pes-ARKO derived mice with restored AR function due to the presence of a T857A substitution in the AR gene (pes-ARKO-T857A).

Disclosed are methods of generating a cell line wherein the AR loci has been disrupted. For example, the AR loci can be disrupted by, for example, disrupting one of the exons, such that a stop codon terminates translation of the AR peptide early or where the exon is completely taken out. The AR loci would include any exon or intron associated with the AR gene on the X chromosome.

The AR gene is considered any sequence associated with the AR locus. Thus, it would at least include the chromosomal nucleic acid contained within any organism that expresses an AR, such as, the introns, exons, 5′ upstream sequence involved with the AR coding and non-coding sequence, and 3′ downstream sequence involved with the AR coding and non coding sequence.

A disrupted AR loci can be any AR loci that does not produce a native AR protein. A disrupted AR loci would also include any AR loci wherein the nucleic acid of the natural AR gene, including exons and introns has been altered. Typically the altering of the AR gene will cause a disruption in AR function, by for example, preventing DNA binding in the AR gene product or ligand binding in the AR gene product or transactivating activity in the AR gene product. The disrupted AR loci can be made using any known technique, including homologous recombination techniques. The disrupted loci can be an alteration of any exon to produce a non-functional AR protein. Furthermore, disclosed are constructs and methods to mutate any exon in the AR through homologous recombination via the surrounding introns. For example, Exon 1 can be floxed through addition of a lox site in sequence that will homologously recombine with Intron 1 and intron 2. Likewise lox sites could be inserted into sequence which would homologously recombine with intron 2 and intron 3 for Exon 2, intron 3 and intron 4 for exon 3, intron 4 and intron 5 for exon 4, intron 5 and intron 6 for exon 5, and so forth for each exon which are considered disclosed herein.

The disrupted AR loci can be in any cell that contains an AR loci, such as an embryonic stem cell, an embryonic germ cell, a breast cell, a breast cancer cell, an ovary cell, an ovary cancer cell, and any cell line of cells that contain AR genes which are expressed, such as prostate cells, testis, bone, brain, neural, and muscle.

Disclosed are methods of generating an animal wherein the AR loci has been disrupted a) wherein the disruption is tissue-specific, b) wherein sequence associated with the AR loci is flanked by sites which can be acted upon a recombinase, such as loxP sites, and c) wherein the sites can be cleaved by a recombinase, such as cre recombinase, under the control of an tissue specific promoter such as, the probasin promoter.

Also disclosed are methods wherein the cre recombinase is under the control of a promoter specific for breast tissue, such as the WAP promoter, a promoter specific for ovarian tissue, such as the ACTB promoter, a promoter specific for bone tissue. Any tissues specific promoter can be used. Promoters specific for prostate, testis, and neural are also disclosed.

Disclosed are inducible expression systems to generate mice without a functional androgen receptor. It is understood that many inducible expression systems exist in the art and may be used as disclosed herein. Inducible expression systems can include, but are not limited to the Cre-lox system, Flp recombinase, and tetracycline responsive promoters. The Cre recombinase system which when used will execute a site-specific recombination event at loxP sites. A segment of DNA that is flanked by the loxP sites, floxed, is excised from the transcript. To create null mice using the Cre-lox system, two types of transgenic mice are created. The first is a mouse transgenic for Cre recombinase under control of a known inducible and/or tissue-specific promoter. The second is a mouse that contains the floxed gene. These two transgenic mouse strains are then crossed to create one strain comprising both mutations. Disclosed are constructs and mice that place the androgen receptor (AR) gene in the floxed position such that upon recombination an AR null mutation is created. Control of the recombination event, via the Cre Recombinase, can be constitutive or inducible, as well as ubiquitous or tissue specific, depending on the promoter used to control Cre expression. Disclosed is a constitutive system in which the Cre recombinase is expressed from a β-actin promoter. Other inducible expression systems exist and can be used as disclosed herein. Disclosed herein, a non-tissue specific promoter, β-actin, is used in the form of the FVB/N-TgN(ACTB-Cre)2Mrt (stock #003376) mice (Jackson Laboratory, Bar Harbor, Me.). However, the CMV promoter and adenovirus EIIa promoter, for example, are also examples of ubiquitous promoters and can be substituted for β-actin to achieve the same result. Also disclosed are constructs and their use comprising the WAP promoter for the establishment of an inducible AR null mutation. Herein, B6129-TgN(WAPCre) 11738Mam (stock #003552) (Jackson Laboratory, Bar Harbor, Me.) mice are used to establish tissue-specific Cre recombinase expression, with Cre under the control of WAP. It is understood that other expression systems may be substituted for the Cre expression system disclosed herein. It is anticipated that variations in the expression system used can result in a need to change other components of the recombination event, for example, the promoter. Commercially available mice (Jackson Laboratory, Bar Harbor, Me.) that utilize the cre-lox inducible expression system include at least 129-TgN(PRM-Cre)58Og (stock #003328), 129.Cg-Foxg1^{tml(Cre)Skm} (stock #004337), 129S6-Tg(Pmp-GFP/Cre) 1 Blw (stock #003960), B6.129-Tg(Pcp2-Cre)2Mpin (stock #004146), B6.129S4-Meox2^{CreSor} (stock #003755), B6.Cg(D2)-TgN(xstpxLacZ)32And (stock #002982), B6.Cg(SJL)-TgN(NesCre)1Kln (stock #003771), B6.Cg-Tg(Rbp3-Cre)528Jxm (stock #003967), B6-Cg-Tg(Syn1-Cre)671Jxm (stock #003966), B6.Cg-Tg(Tek-Cre)12Flv (stock #004128), B6.Cg-TgN(LckCre)548Jxm (stock #003802), B6.FVB-TgN(EIIa-Cre)C5379Lmgd (stock #003724), B6129-TgN(MMTV-Cre)1Mam (stock #003551), B6129-TgN(MMTV-Cre)4Mam (stock #003553), B6129-TgN(WAPCre)11738Mam (stock #003552), B6; D2-TgN(Sycp1-Cre)4 Min (stock #003466), B6; FVB-TgN(GZMB-Cre)1Jcb (stock #003734), B6; SJL-TgN(Co12a1-Cre)1Bhr (stock #003554), BALB/c-TgN(CMV-Cre)#Cgn (stock #003465), C.129P2-Cd19^{tml(Cre)Cgn} (stock #004126), C57BL/6-TgN(AlbCre)21Mgn (stock #003574), C57BL/6-TgN(Ins2Cre)₂₅Mgn (stock #003573), C57BL/6-TgN(Zp3-Cre)3Mrt (stock #003394), C57BL/6-TgN(Zp3-Cre)93Knw (stock #003651), C57BL16-TgN(Mxl-Cre)1Cgn (stock #003556), DBA/2, TgN(xstpxLacZ)36And (stock #002981), FVB/N-TgN(ACTB-Cre)2Mrt (stock #003376), FVB/N-TgN(EIIa-Cre)C5379Lmgd (stock #003314), FVB/N-TgN(Zp3-Cre)3Mrt (stock A 003377), STOCK Mttp^{tmlSgy}Ldlr^{tmlSgy}Apob^{tmlSgy}Tg(Mx-Cre)1Cgn (stock #004192), STOCK TgN(Wnt1-GAL4)11Rth (stock #003829), STOCK TgN(Wnt1-Cre)11Rth (stock #003829), STOCK TgN(balancer1)2Cgn (stock #002858), STOCK TgN(balancer2)1Cgn (stock #002859), and STOCK TgN(hCMV-Cre)140Sau (stock #002471). Among these mice, B6.Cg(SJL)-TgN(NesCre)1Kln (stock #003771), B6.Cg-Tg(Syn1-Cre)671Jxm (stock #003966), and C57BL/6-TgN(Ins2Cre)25Mgn (stock #003573) are examples of mice that have tissue specific Cre promoters. The B6.Cg-TgN(LckCre)548Jxm (stock #003802) mice place Cre under control of the Lck promoter and do not have tissue specificity. The B6.FVB-TgN(EIIa-Cre)C5379Lmgd (stock #003724) and BALB/c-TgN(CMV-Cre)#Cgn (stock #003465) also have Cre recombinase under the control of a non-tissue-specific promoter. The disclosed floxed AR mice may be crossed with any of the Cre mice available to take advantage of additional promoter activity and specificity. Commercially available mice (Jackson Laboratory, Bar Harbor, Me.) that utilize the Flp recombinase expression system are 129S4/SvJaeSor-Gt(ROSA)26Sor^{tml(FLP1)Dym} (stock #003946) and B6; SJL-TgN(ACTFLPe)9205Dym (stock #003800). Also disclosed are the Offspring of the disclosed floxed AR mice crossed with the disclosed Cre mice. Thus, for example, are the AR knock-out mice (ARKO) mice (i.e., pes-ARKO-TRAMP, ind-ARKO-TRAMP, and tgn-ARKO) disclosed herein.

Thus, disclosed herein are transgenic mammals comprising a disrupted AR gene, wherein the disrupted gene is produced by action of a recombinase operably linked to a tissue specific promoter. It is understood that the tissue specific promoter can be a prostate epithelial specific promoter. For example, disclosed herein are transgenic mammal wherein the tissue specific promoter is an epithelial prostate specific promoter selected from the group consisting of probasin, prostatic promoter, secretory protein-94 (PSP94) promoter, and Nkx3.1 promoter. Also, for example, disclosed herein are transgenic mammal wherein the tissue specific promoter is the stromal prostate specific promoter such as the ARA55 promoter, and the transgelin promoter (SM22 including inducive promoters SM22-rtTA and Tagln-cre). It is understood that the transgenic mammals disclosed herein can be porcine, bovine, murine, primate (human and non-human), rat, guinea pig, and rabbit. Thus, for example disclosed herein are transgenic mice comprising a disrupted AR gene, wherein the disrupted gene is produced by action of a recombinase operably linked to a tissue specific promoter. It is understood and herein contemplated that the tissue specific promoter can be a epithelial specific or stromal specific promoter. Alternatively, it is understood and herein contemplated that the tissue specific promoter can be a prostate specific promoter that does not distinguish between epithelial and stromal cells.

It is also understood that if a particular AR gene is disclosed herein, specifically disclosed is each an every species variant of that AR gene. Thus for example disclosed herein are transgenic mice comprising a disrupted AR gene, wherein the disrupted gene is produced by action of a recombinase operably linked to a tissue specific promoter, and wherein the AR gene is a murine gene. It is also contemplated herein that the disclosed transgenic mice can be chimeric for a given gene. Thus, for example, the disclosed transgenic mice can comprise a disrupted human AR gene operably linked to a tissue specific promoter.

The disclosed transgenic mammals can also comprise “knock-in” mutations to the disrupted AR gene to restore function to the AR knock-out. For example, the substitution of Alanine for Threonine at residue 857 of murine AR gene results in the constitutively active AR similar to the substitution of Alanine for Threonine at residue 877 of the human AR gene. It is understood that the resulting transgenic animals do not lose AR expression over time due to the point mutation resulting in the amino acid substitution.

As noted above the disruption of the AR gene can result in any number of ways known in the art. For example, disclosed herein are disrupted AR genes, wherein the disrupted gene comprises a mutation in the AR gene such as a missense or nonsense mutation. Also disclosed are disrupted AR genes, wherein the AR is disrupted through the insertion of a gene cassette or reporter gene such as neomycin. Thus, disclosed herein are transgenic animals comprising a disrupted AR gene, wherein the AR gene comprises a gene cassette, missense mutation, or nonsense mutation.

The disclosed transgenic animals have disrupted AR gene expression through the presence of a mutation or insertion that is flanked by loxP sites such that upon expression of cre recombinase, the mutation or insertion is excised from the gene permitting full expression of AR. It is understood that by operably linking cre recombinase to a tissue-specific promoter, the knock-out phenotype is limited to a particular tissue. For example, the disclosed transgenic animals comprising cre recombinase under the control of a probasin promoter only lose expression of AR in the prostate epithelia. It also is understood that as constructed expression of the promoter controlling cre expression results in the expression of a functional AR gene as the cre recombinase will cut out the disrupted area of the AR gene at the loxP sites. Loss of promoter expression will leave the loxP sites intact and thus Ar function is lost. Thus, for example, as probasin expression is lost AR expression decreases.

It is understood that the disclosed transgenic animals can be crossed with other transgenic animals to establish a new transgenic animal with the features of both parents. For example, disclosed herein are transgenic animals resulting from the cross of pes-ARKO mice with TRAMP mice resulting in a pes-ARKO-TRAMP mouse. Such transgenic mice can be used as models for the study of cancer progression.

Disclosed herein are cells, wherein the cell has a disrupted AR gene, wherein the disrupted gene is produced by action of a recombinase operably linked to a tissue specific promoter. It is understood and herein contemplated that the cell can be an embryonic stem cell, an embryonic germ cell, a breast cell, a breast cancer cell, an ovary cell, an ovary cancer cell, a prostate cell, a testis cell, a bone cell, a brain cell, a neural cell, or a muscle cell. It is also understood that the cell can be derived from a cancer, for example, a prostate cancer cell obtained from a subject or prostate cancer cell line.

The cells disclosed herein can comprise inducible expression systems such as the cre-lox system. It is also understood that AR expression in the cells disclosed herein can be tissue specific. One way known to achieve the tissue specific expression of AR is to disrupt the AR gene by creating a missense or nonsense mutation in the AR gene or disrupting the gene through the insertion of a gene cassette. By flanking the insertion or mutation in the AR gene with loxP sites and placing cre recombinase under the control of a tissue specific promoter, the resulting cell will only express AR when the tissue specific promoter is expressed which will drive cre recombinase expression and remove the disruption in the AR gene at the loxP sites. It is understood that the tissue specific promoter can be a prostate epithelial promoter selected from the group consisting of probasin promoter, prostatic secretory protein-94 (PSP94) promoter, and Nkx3.1 promoter. Thus for example disclosed herein are cells comprising a disrupted AR gene wherein the AR gene is disrupted by the presence of a gene cassette inserted into the AR gene and flanked by loxP sites; wherein the cell further comprises cre recombinase operably linked to a probasin promoter.

Also disclosed herein are cells comprising point mutations that restore function to a cell comprising a knock out transgene. The “knock-in” can be, for example, a Threonine to Alanine substitution. Thus, disclosed herein are cells comprising a disrupted AR gene under control of a cre-lox system, further comprising a T857A substitution of the mouse AR gene. Also disclosed are cells comprising a disrupted AR gene under control of a cre-lox system, further comprising a T877A substitution of the human AR gene. It is understood and herein contemplated that the AR gene of the cells disclosed herein can be from any mammalian source. Thus, for example, disclosed herein are cells comprising a disrupted AR gene, wherein the AR gene is a murine AR gene. Also disclosed are disrupted human AR genes.

Disclosed are mammals comprising the vector and/or cells disclosed herein. For example disclosed herein is a mammal comprising a cell, wherein the cell has a disrupted AR gene, wherein the disrupted gene is produced by action of a recombinase operably linked to a tissue specific promoter. It is understood that the disclosed mammals can be transgenic for other genes. Thus, for example, disclosed are mammals comprising the cells disclosed herein further comprising a transgene for adenocarsinoma of mouste prostate (TRAMP). Thus, for example, disclosed herein are pes-ARKO-TRAMP and ind-ARKO-TRAMP mice.

Disclosed are mammals, wherein the mammal is bovine, ovine, porcine, primate (including human and non-human primates), murine (mouse), rat, hamster, or rabbit.

Also disclosed herein are cells, wherein the cells are an Androgen Receptor (AR)-negative prostate metastatic cell, and wherein the cell is stably transfected with an AR gene under the control of an AR promoter. For example, disclosed herein are PC-3 cells stably transfected with an AR gene under the control of an AR promoter (i.e., PC-3(AR)9 cells).

Also disclosed herein are cells, wherein the cells are an AR-positive prostate metastatic cell, and wherein the cell has AR expression “knocked down.” For example, disclosed herein is a cell that is stably transfected with an AR siRNA. For example disclosed herein are WPMY1 cells stably transfected with an AR siRNA (i.e., WPMY1-ARsi cells). It is understood that an alternative method for creating an AR “knock-down” is through gene recombination. Thus, disclosed herein is a cell with knocked-down AR expression. For example, disclosed herein are CWR22R-AR+ cells with recombinantly knocked down AR (i.e., CWR22R-AR^{+/-} cells).

### 2. Homology/Identity

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

It is understood that one way to define any known variants and derivatives or those that might arise, of the disclosed genes and proteins herein is through defining the variants and derivatives in terms of homology to specific known sequences. For example SEQ ID NO: 9 sets forth a particular sequence of an AR gene and SEQ ID NO: 8 sets forth a particular sequence of the protein encoded by SEQ ID NO: 9, an AR protein. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### 3. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### 4. Nucleic Acids

There are a variety of molecules disclosed herein that are nucleic acid based, including for example the nucleic acids that encode, for example AR, or any of the nucleic acids disclosed herein for making AR knockouts, or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to the protein molecules involved in the signaling pathways disclosed herein, all of which are encoded by nucleic acids or are nucleic acids. The sequences for the human analogs of these genes, as well as other analogs, and alleles of these genes, and splice variants and other types of variants, are available in a variety of protein and gene databases, including Genbank. Those sequences available at the time of filing this application at Genbank are herein incorporated by reference in their entireties as well as for individual subsequences contained therein. Genbank can be accessed at http://www.ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

c) Primers and Probes

Disclosed are compositions including primers and probes, which are capable of interacting with the disclosed nucleic acids, such as the AR, probasin, Nkx3.1, and prostatic secretory protein-94 (PSP94) as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the AR gene typically will be used to produce an amplified DNA product that contains a region of the AR gene or the complete gene. In general, typically the size of the product will be such that the size can be accurately determined to within 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

### 5. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as AR into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase m transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Venna, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed AR or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### 6. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fingi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### 7. Peptides

a) Protein Variants

As discussed herein there are numerous variants of the AR protein that are known and herein contemplated. In addition, to the known functional AR strain variants there are derivatives of the AR proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 3
Amino Acid Abbreviations

| Amino Acid | Abbreviations | |
|---|---|---|
| alanine | Ala | A |
| allosoleucine | AIle | |
| arginine | Arg | R |
| asparagine | Asn | N |
| aspartic acid | Asp | D |
| cysteine | Cys | C |
| glutamic acid | Glu | E |
| glutamine | Gln | K |
| glycine | Gly | G |
| histidine | His | H |
| isolelucine | Ile | I |
| leucine | Leu | L |
| lysine | Lys | K |
| phenylalanine | Phe | F |
| proline | Pro | P |
| pyroglutamic acidp | Glu | |
| serine | Ser | S |
| threonine | Thr | T |
| tyrosine | Tyr | Y |
| tryptophan | Trp | W |
| valine | Val | V |

TABLE 4
Amino Acid Substitutions

| Original | Exemplary Conservative Substitutions, |
|---|---|
| Residue | others are known in the art. |
| Ala | ser |
| Arg | lys, gln |
| Asn | gln; his |
| Asp | glu |
| Cys | ser |
| Gln | asn, lys |
| Glu | asp |
| Gly | pro |
| His | asn; gln |
| Ile | leu; val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 4, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:9 sets forth a particular sequence of AR and SEQ ID NO:8 sets forth a particular sequence of a AR protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO:8 is set forth in SEQ ID NO:9. It is also understood that while no amino acid sequence indicates what particular DNA sequence encodes that protein within an organism, where particular variants of a disclosed protein are disclosed herein, the known nucleic acid sequence that encodes that protein in the particular AR from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 3 and Table 4. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

### 8. Antibodies

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with AR. Antibodies that bind the disclosed regions of AR involved in the interaction between AR and Androgen are also disclosed. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(2) Human Antibodies

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art.

For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### 9. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as a vector comprising an AR, for treating, inhibiting, or preventing a cancer (e.g., prostate cancer), the efficacy of the therapeutic vector can be assessed in various ways well known to the skilled practitioner. For instance, one of ordinary skill in the art will understand that a composition, such as vector comprising AR, disclosed herein is efficacious in treating or inhibiting an prostate cancer in a subject by observing that the composition reduces tumor growth rate or prevents a further increase in tumor size.

The compositions disclosed herein may be administered prophylactically to patients or subjects who are at risk for a cancer, for example prostate cancer.

### 10. Compositions Identified by Screening with Disclosed Compositions

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, AR, are also disclosed. Thus, the products produced using the molecular modeling approaches that involve the disclosed compositions, such as, AR, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol._{—}Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

### 11. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for assessing a subject's risk for acquiring prostate cancer, comprising the oligonucleotides set forth in SEQ ID Nos: 11 and 12.

### 12. Compositions with Similar Functions

It is understood that the compositions disclosed herein have certain functions, such as enhancing epithelial AR expression. Disclosed herein are certain structural requirements for performing the disclosed functions, and it is understood that there are a variety of structures which can perform the same function which are related to the disclosed structures, and that these structures will ultimately achieve the same result, for example stimulation or inhibition epithelial AR expression.

### E. Methods of Making the Compositions

The compositions disclosed herein and the compositions necessary to perform the disclosed methods can be made using any method known to those of skill in the art for that particular reagent or compound unless otherwise specifically noted.

### 1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System IPlus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

### 2. Peptide Synthesis

One method of producing the disclosed proteins, such as SEQ ID NO: 8, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

### 3. Process Claims for Making the Compositions

Disclosed are processes for making the compositions as well as making the intermediates leading to the compositions. For example, disclosed are nucleic acids in SEQ ID NOs: 9. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth in SEQ ID NO: 9 and a sequence controlling the expression of the nucleic acid.

Also disclosed are nucleic acid molecules produced by the process comprising linking in an operative way a nucleic acid molecule comprising a sequence having 80% identity to a sequence set forth in SEQ ID NO: 9, and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence set forth in SEQ ID NO: 9 and a sequence controlling the expression of the nucleic acid.

Disclosed are nucleic acid molecules produced by the process comprising linking in an operative way a nucleic acid molecule comprising a sequence encoding a peptide set forth in SEQ ID NO: 9 and a sequence controlling an expression of the nucleic acid molecule.

Disclosed are nucleic acid molecules produced by the process comprising linking in an operative way a nucleic acid molecule comprising a sequence encoding a peptide having 80% identity to a peptide set forth in SEQ ID NO: 9 and a sequence controlling an expression of the nucleic acid molecule.

Disclosed are nucleic acids produced by the process comprising linking in an operative way a nucleic acid molecule comprising a sequence encoding a peptide having 80% identity to a peptide set forth in SEQ ID NO: 8, wherein any change from the in SEQ ID NO: 8 are conservative changes and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Increased Prostate Cell Proliferation and Loss of Cell Differentiation in Mice Lacking Prostate Epithelial Androgen Receptor

a) Generation and Characterization of Pes-ARKO Mice

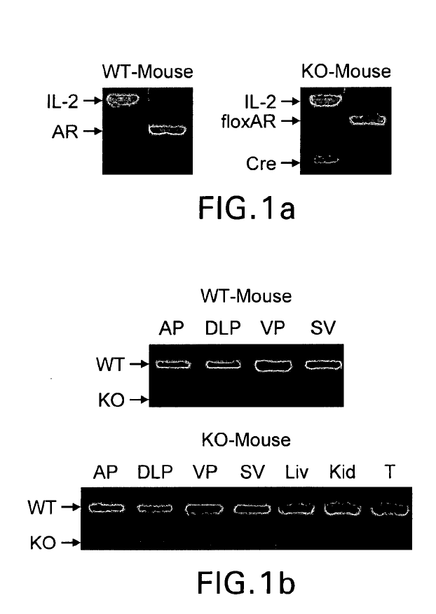

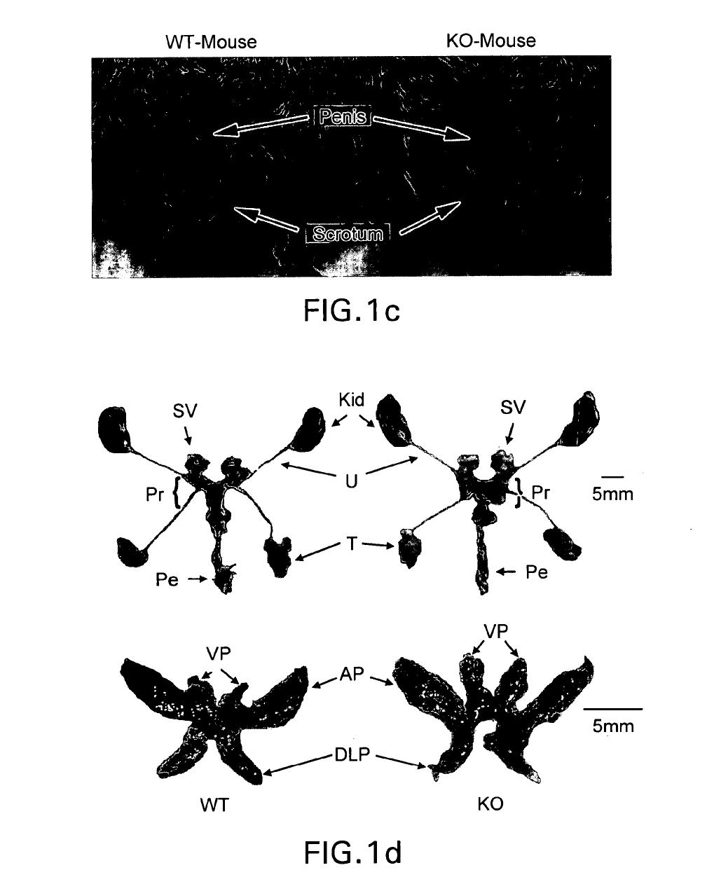

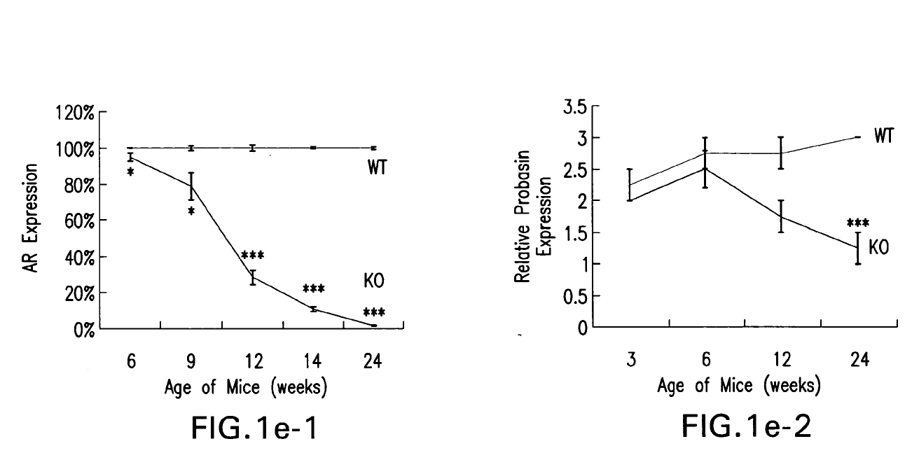

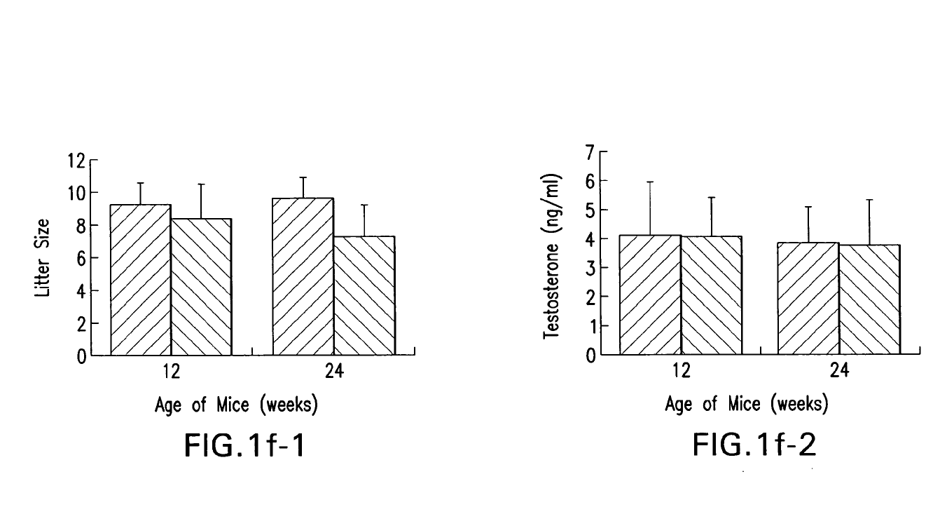

Pes-ARKO mice contain a prostate epithelial specific promoter (Greenberg, N. M. et al. (1994) Mol Endocrinol 8, 230-9) driving cre-recombinase. Expression of the probasin promoter transgene has been reported to be increasingly expressed from 2-7 weeks and sustained expression is observed throughout life (Wu, X. et al. (2001) Mech Dev 101, 61-9). To verify AR gene deletion within prostate epithelium of pes-ARKO mice, candidate mice were genotyped for probasin-cre transgene and conditional flox-AR allele (FIG. 1a). The specificity of recombination was also evaluated in several key organs by RT-PCR using primers directed towards exons 1 and 3 of the AR gene. Deletion of AR exon 2 was confirmed by the detection of truncated transcripts via RT-PCR present within the ventral prostate and dorsal-lateral prostate of pes-ARKO mice only (FIG. 1b). This lobe specific expression is consistent with the probasin promoter transgene driven expression in other models (Greenberg, N. M. et al. (1995) Proc Natl Acad Sci USA 92, 3439-43). No other tissues in wild-type (WT) or pes-ARKO mice contained truncated forms of AR DNA.

There were no differences in external characteristics, including genital-anal distances between WT and pes-ARKO mice (FIG. 1c). The internal urogenital organs also showed no differences between WT and pes-ARKO mice (FIG. 1d, upper panels). In contrast, pes-ARKO mice had significantly larger ventral prostates at week 24 (FIG. 1d, lower right). Neither the dorso-lateral prostate nor anterior prostate within pes-ARKO mice significantly changed in size.

The progressive loss of AR was confirmed by immunohistochemistry. A labeling index for epithelial AR was determined and tabulated (FIG. 1e, left). AR protein localized to epithelial nuclei slowly decreased with age in pes-ARKO mice compared to WT littermates. By week 24 epithelial AR detection was rare. To indirectly evaluate epithelial AR signaling, the staining intensity of probasin, an androgen regulated protein in mature animals, was quantified. Probasin intensity was similar in pes-ARKO and WT littermates until 12 weeks of age, when the reduced level in pes-ARKO samples compared to WT approached significance (P<0.057). By week 24 and thereafter, this difference was significant (P<0.027) (FIG. 1e, right). These data indicate that probasin expression is normal before significant loss of AR and epithelial AR decline precedes the loss of an AR-dependent secreted protein.

To determine if pes-ARKO mice contain abnormalities other than enlarged ventral prostates, fertility was evaluated. There were no significant differences in litter-size when either WT or pes-ARKO males were mated to WT females (FIG. 1f). To rule out the possibility of altered ventral prostate size might be due to circulating androgen levels, serum testosterone levels were measured by ELISA. No difference was observed between the WT and pes-ARKO males at 12 or 24 weeks of age (FIG. 1f, right). Together, results shown in FIG. 1 demonstrate an effective deletion of the AR that is confined to the prostatic epithelium and that consequences of the AR gene deletion appears to be restricted to the prostate and are without the influence of serum testosterone.

b) Loss of Differentiated Glandular Structure in Pes-ARKO Mice

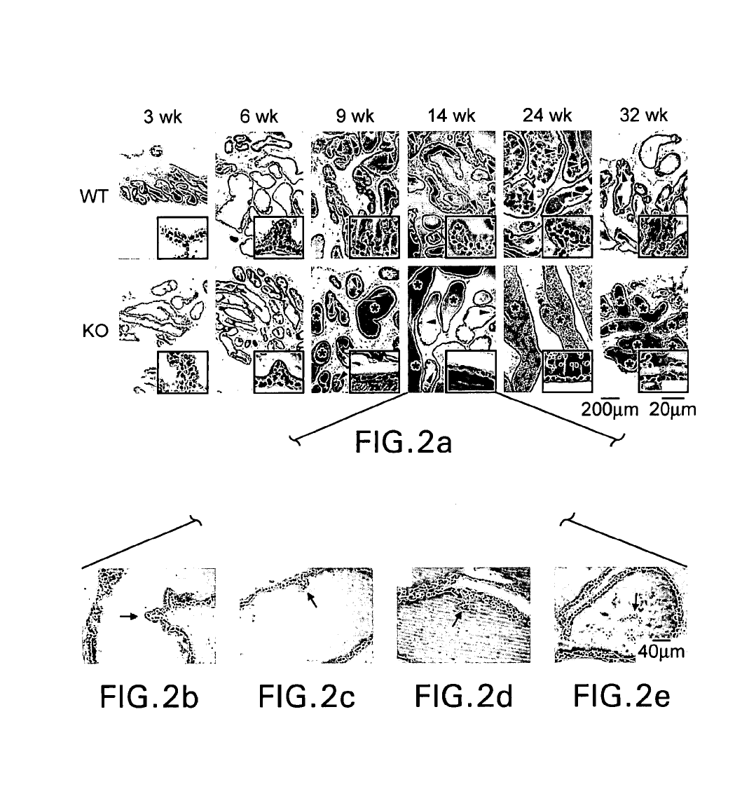

The histomorphology of prostates was checked weekly from 2 through 6 weeks of age and then biweekly thereafter until 32 weeks for the anterior-, dorso-lateral-, and ventral-prostates. Early on, the pes-ARKO glands looked normal, showing considerable prostatic budding, and differentiation into tall, columnar glandular epithelium (FIG. 2). Starting with the inspection at week 9, a progressive loss of this differentiated state was observed in ventral prostates. Notably, there was a loss of glandular infolding, and the epithelial cells now appeared short and cuboidal, and lacked typical features of polarized secretory epithelial cells (FIG. 2).

The induction of prostatic budding in ventral prostate of pes-ARKO mice developed normally and prostatic epithelium reached maturity, containing a high secretory columnar epithelium with glandular infoldings at 5-6 weeks of age for both pes-ARKO mice and WT littermates (FIG. 2a). In pes-ARKO mice at 9 weeks of age some ducts within the ventral prostate contained epithelia that were shorter in height or low cuboidal as compared to taller or columnar epithelia from WT littermates. Concurrent with loss of prostatic epithelial morphology was the loss of glandular infolding in the pes-ARKO mice (FIG. 2a). These changes in histo-morphology increased over time until at 24 weeks (and later) nearly all ducts contained a dedifferentiated epithelium of low height and lack of glandular infolding (FIG. 2a).

In pes-ARKO mice 14 weeks of age an increased number of cells were found as detached layers within the prostatic lumen (FIG. 2e). In ducts where histological dedifferentiation appeared, normal glandular infolding could be observed (FIG. 2b) as well as a range of infoldings with constrictions at their base (FIGS. 2 c,d). As the infoldings became narrower at their base, cells appeared to have lost polarity, as observed by nuclei moving from basal to apical location. Ultimately glandular infoldings were lost, and apparently sloughed into the prostatic lumen (FIG. 2e).

c) Loss of Epithelial AR Decreases Androgen Regulated Gene Expression.

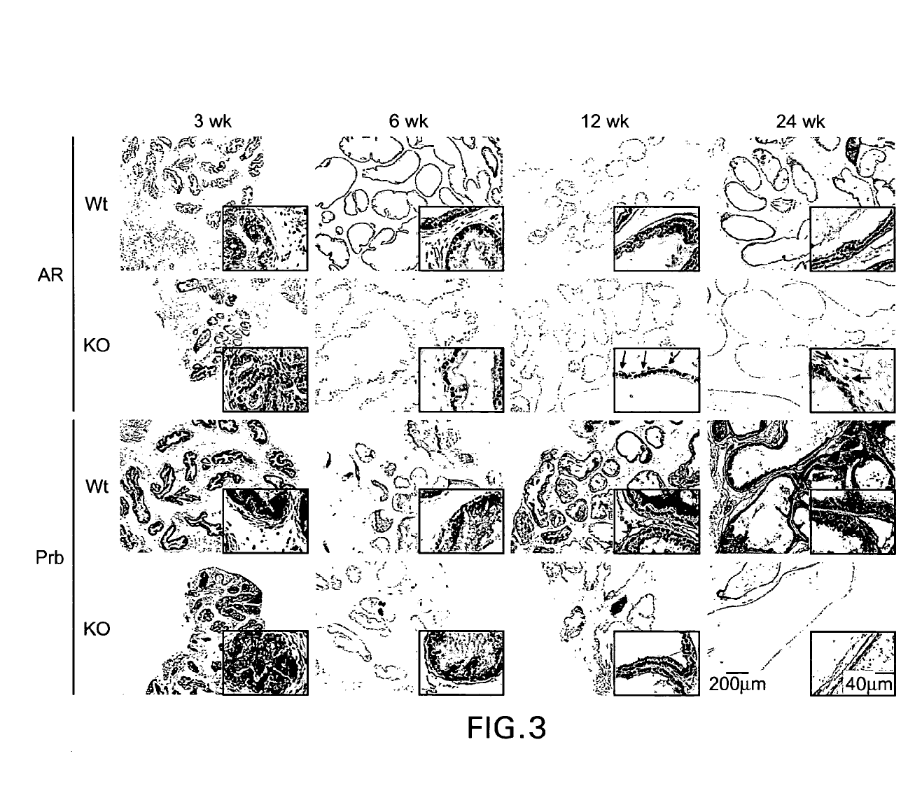

Ventral prostates from WT and pes-ARKO mice of different ages were stained for AR and androgen regulated probasin (FIG. 3). At week 3, AR staining in both epithelial and stromal cells was evident in the two strains. At week 6 the pes-ARKO prostates start to have noticeably weaker epithelial AR staining and by week 24 AR staining in the epithelium seemed gone. There were also a small percentage of epithelial cells within the dorsal-lateral prostate of pes-ARKO mice that lacked AR protein, however nearly all luminal cells of the anterior prostate were positive for AR. The decline of probasin staining in ventral prostate epithelium of the pes-ARKO mice lagged behind that of AR, but was also gone by week 24. (FIG. 3). Importantly, stromal AR was seen at all stages evaluated.

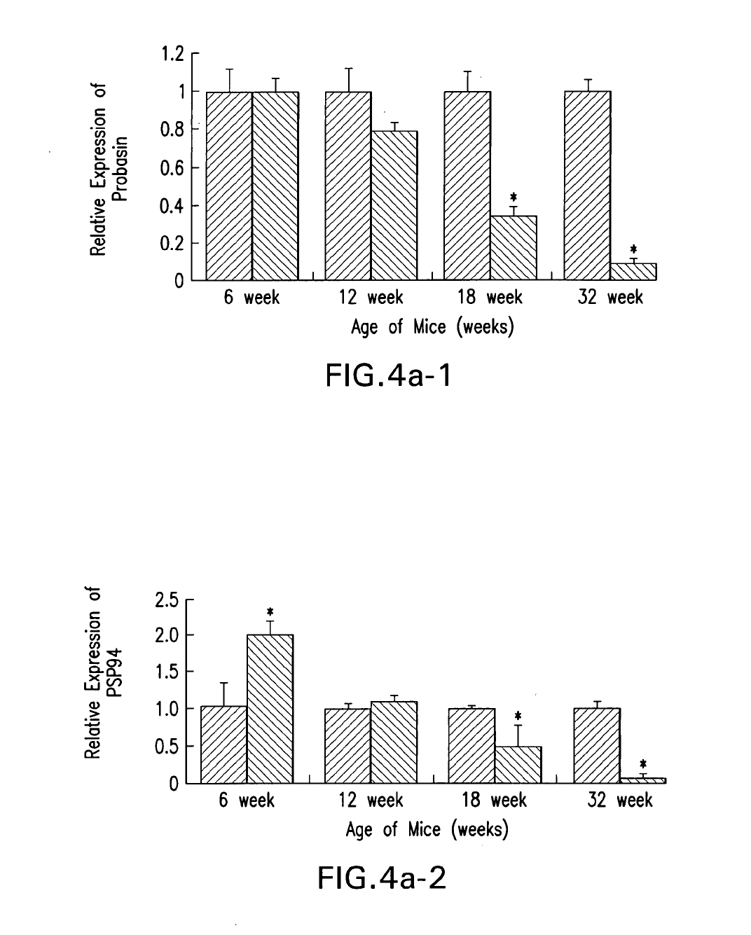

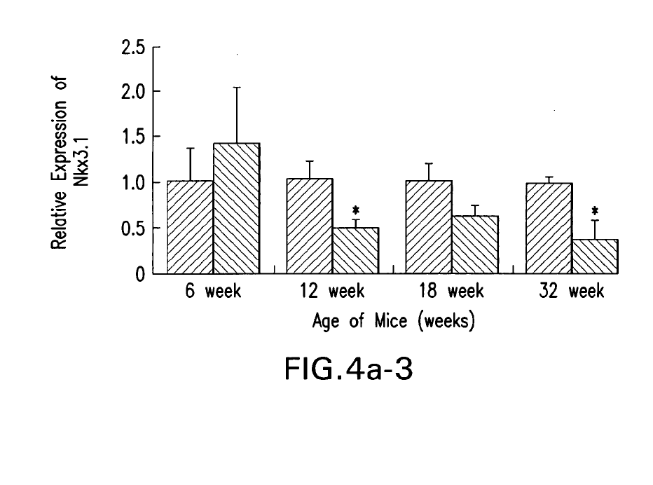

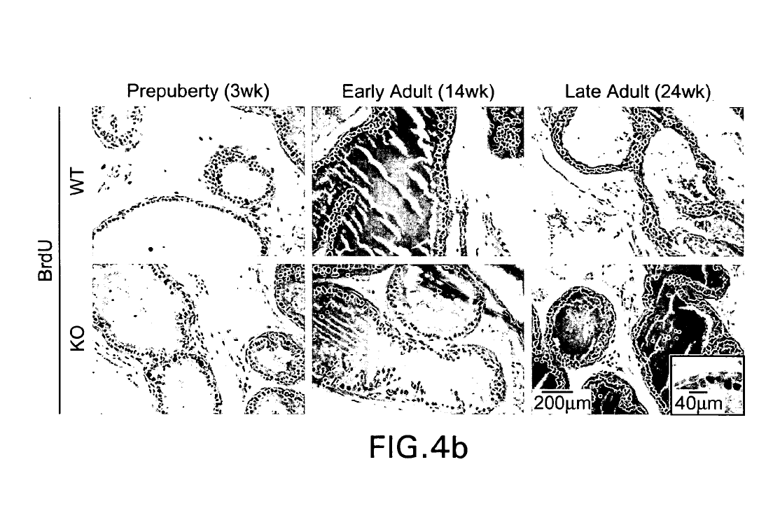

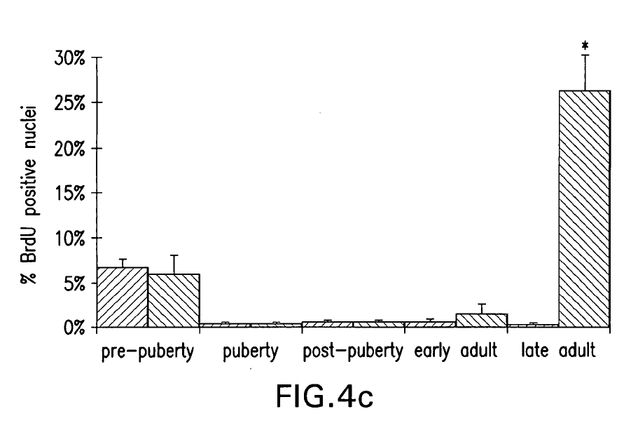

Probasin, prostatic secretory protein-94 (PSP94), and Nkx3.1 are three prostate-specific protein known to be transcriptionally regulated by androgens (He, W. W. et al. (1997) Genomics 43, 69-77; Kwong, J., (2000) Endocrinology 141, 4543-51). However, it is unknown if stromal AR or epithelial AR is responsible for their regulation. To evaluate loss of epithelial AR signaling on downstream gene expression, RT-PCR was performed on ventral prostate RNA from WT and pes-ARKO mice at weeks 6, 12, 18, and 32. In pes-ARKO ventral prostates transcriptional down-regulation of probasin and PSP94 were observed by week 18 and remained lower through the final time point at week 32 (FIG. 4a). Nkx3.1 is a transcription factor that governs prostate morphogenesis and patterning (Bhatia-Gaur, R. et al. (1999) Genes Dev 13, 966-77) as well as a marker in tumor initiation and progression(Kim, M. J. et al. (2002) Cancer Res 62, 2999-3004). Nkx3.1 gene expression is significantly (P<0.05) decreased by 12 weeks and remains low through 32 weeks (FIG. 4a). The decreased expression of Nkx3.1 coincides with the marked loss of glandular infolding. Thus, AR signaling is lost significantly within prostate epithelia by week 12 and that epithelial AR is the major transcriptional regulator of these genes. These data agree with the report suggesting that epithelial AR governs secretory protein expression (Donjacour, A. A. & Cunha, G. R. (1993) Endocrinology 132, 2342-50) and indicate that loss of epithelial AR signaling leads to both biochemical and structural dedifferentiation of the mature prostate.

d) Mature Prostate Growth is Increased in Pes-ARKO Mice

Tissue growth occurs through hypertrophy and/or hyperplasia, and is balanced by cell death. The normal adult prostate is growth-quiescent, whereas in prostate disease organ size and epithelial proliferation increases. Ventral prostates of the pes-ARKO mice were larger than those of their WT littermates. As noted in the histological examination of ventral prostates from pes-ARKO mice, epithelial cells shrunk in size, indicating that ventral prostate enlargement was not due to hypertrophy (FIG. 2a 14-32 wk). To check for ventral prostate proliferation, bromo-deoxyuridine (BrdU) incorporation was evaluated. In prepubescent and mature prostates up to week 14 regardless of strain, BrdU incorporation was not different and was primarily localized to epithelial cell nuclei (FIG. 4b). However, by week 24, concurrent with nearly complete loss of epithelial AR, BrdU incorporation was significantly higher in prostatic epithelium of pes-ARKO mice than in WT littermates (FIG. 4 b and c). This finding was confirmed with an immunohistochemical test for proliferating cell nuclear antigen (PCNA). It has been widely accepted that prostatic epithelial cell proliferation is maintained by androgen-regulated stromal factors (Chang, C. S., et al. (1988) Science 240, 324-6; Wang, Y. et al. (2001) Cancer Res 61, 6064-72) and by androgens having a direct proliferative effect on epithelial cells (Bello, D., et al. (1997) Carcinogenesis 18, 1215-23; Danielpour, D., et al. (1994) Cancer Res 54, 3413-21). However, it is now evident that epithelial androgen/AR signaling induces production of growth suppressors within the luminal epithelial cells, which act upon the underlying AR-negative progenitor cells to inhibit growth. Alternatively, AR directly or indirectly regulates epithelial or stromal production of growth factors which in turn regulate growth of progenitor cells. Therefore, removal of AR releases growth suppressive effects of the luminal cells and proliferation occurs. The lack of AR within the epithelium of pes-ARKO mice spatially recapitulates what is observed in early prostate development, in that AR is present only within the stroma and not in the epithelium (Shannon, J. M. & Cunha, G. R. (1983) Prostate 4, 367-373; Takeda, H., et al. (1985) J. Endocrinol. 104, 87-92). Interestingly, during this time of development epithelial proliferation is high (Donjacour, A. A. & Cunha, G. R. (1988) Develop. Biol. 128, 1-14; Sugimura, Y., Cunha, G. R. & Donjacour, A. A. (1986) Biol. Reprod. 34, 961-971). The results presented here represent a new concept in prostate biology, in which epithelial AR is capable of controlling epithelial growth by acting as a suppressor of epithelial proliferation in the mature prostate.

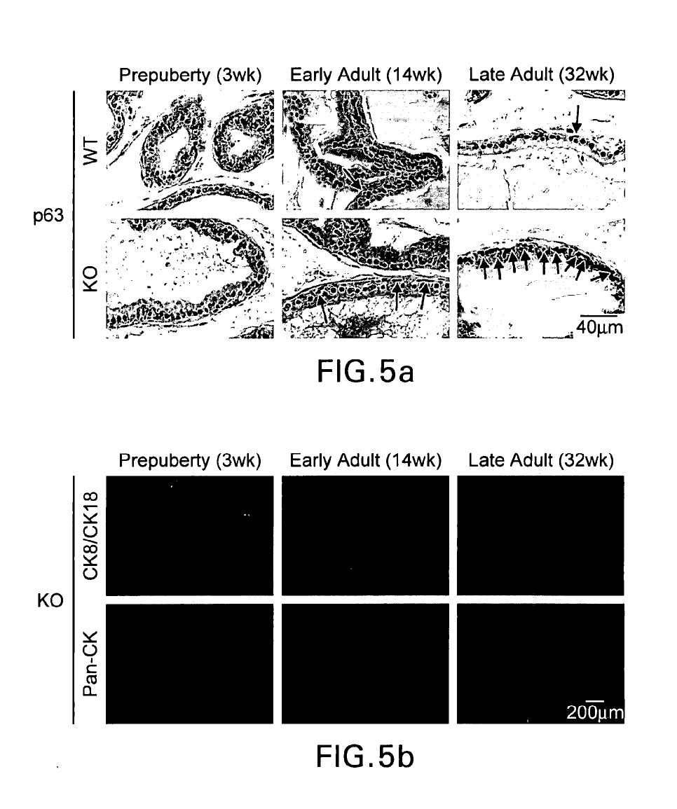

Using immunocytochemical markers for basal and luminal epithelial cells, it was then determined that, as the pes-ARKO animals matured, the p63-positive basal epithelial cell population increased during puberty and then remained elevated while the cytokeratins-8 and -18-positive luminal epithelial cell population declined. In contrast, in the WT animals, the basal cell number declined with age whereas, the luminal cell population remained stable (FIG. 5).

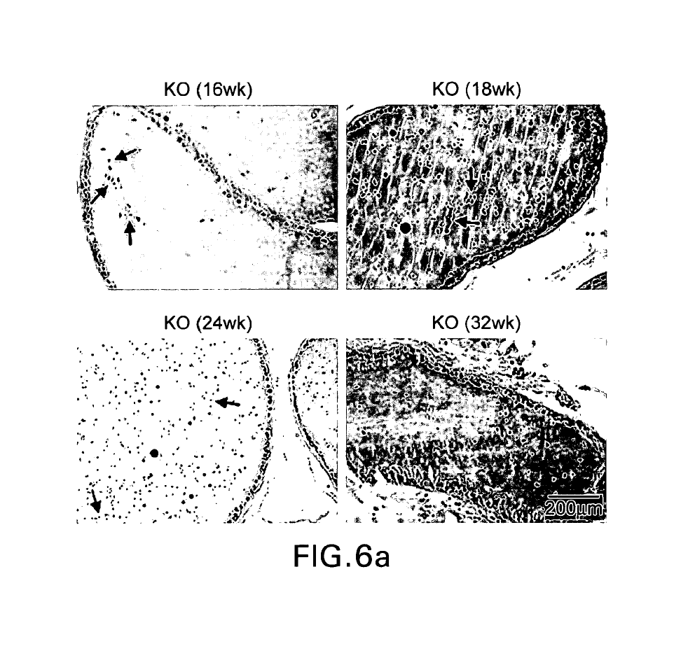

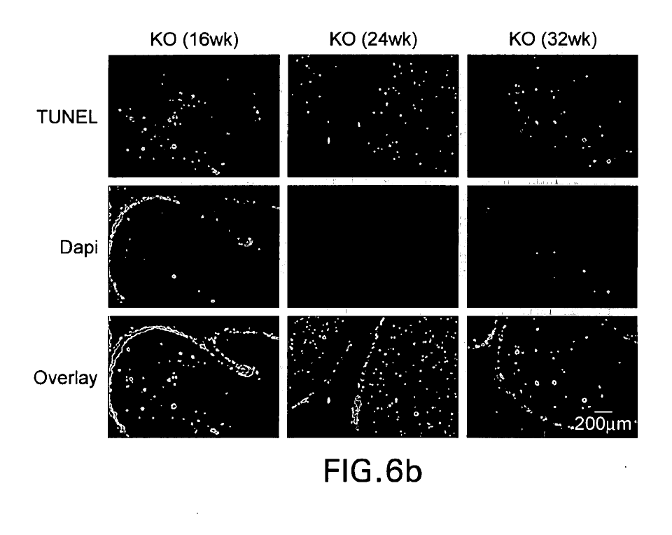

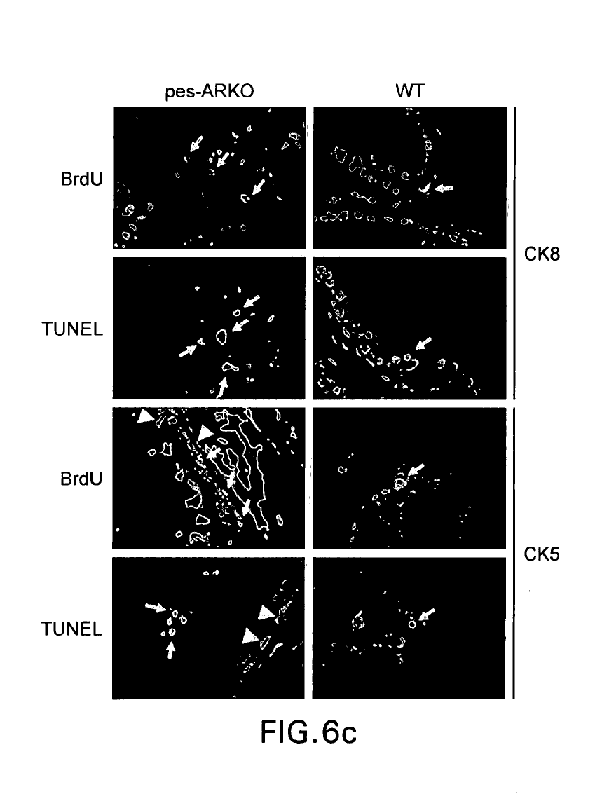

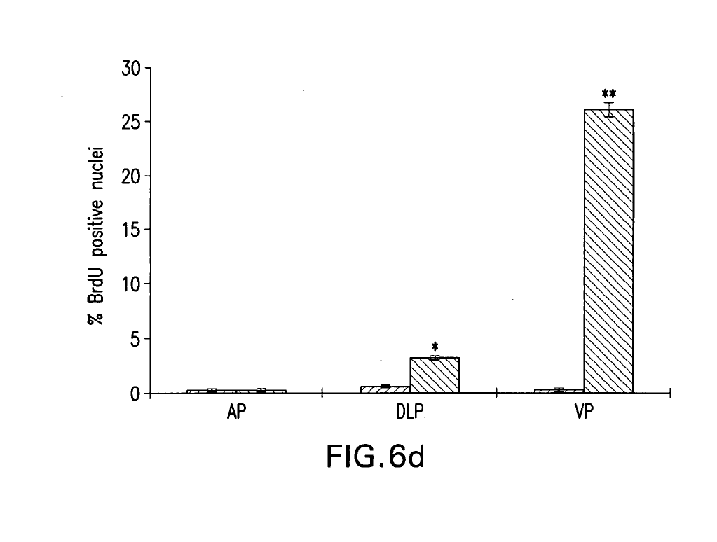

To evaluate which population of epithelial cells increased over time in pes-ARKO mice, each cell type was identified using cell specific markers. Basal and luminal cells were identified histochemically using antibodies directed towards p63 (Signoretti, S. et al. (2000) Am J Pathol 157, 1769-75; Wang, Y., et al. (2001) Differentiation 68, 270-279) and cytokeratins-8 and -18 (Hayward, S. W. et al. (1996) Acta Anat (Basel) 155, 81-93), respectively. Expression of p63 is critical for maintaining the progenitor-cell population that is necessary to sustain epithelial development and morphogenesis (Yang, A. et al. (1999) Nature 398, 714-8; Signoretti, S. & Loda, M. (2006) Cell Cycle 5, 138-41). As expected the number of basal cells decreased over time in WT mice, whereas p63 positive cell numbers remained elevated through 32 weeks of age in pes-ARKO (FIG. 5). BrdU-positive cells were primarily colocalized with CK5-positive basal cells and to a lesser extent with CK8 positive cells (FIG. 6d). Localization of cytokeratins-8 and -18, as well as pan-cytokeratin-positive cells were similar in WT and pes-ARKO through puberty. However, as pes-ARKO mice aged and AR protein expression decreased, expression of cytokeratins-8, -18 and pan-cytokeratin were diminished (FIG. 5).

To evaluate cell death histological and TUNEL staining was used in ventral prostates from pes-ARKO mice (FIG. 6b). However, within the lumen, layers of sloughed epithelium, immune cells and fragmented DNA were observed. TUNEL analysis demonstrated that there were no differences in the apoptotic rates between WT and pes-ARKO ventral prostatic epithelium and stroma. However numerous TUNEL-positive cells or nuclear fragments were observed within the prostatic lumen of pes-ARKO mice. The lack of apoptosis within the epithelial layer was not surprising, since lack of androgen/AR signaling has been shown to be mediated through the stroma. Since there were few apoptotic cells within the intact epithelium, this indicated that TUNEL-positive epithelial cells within the lumen had undergone anoikis (Reddig, P. J. & Juliano, R. L. (2005) Cancer Metastasis Rev 24, 425-39) by detaching from their basement membrane prior to the detection of DNA fragmentation, leading to an accumulation of TUNEL-positive DNA and scavenging immune cells within the lumen. These data demonstrate that lack epithelial AR/signaling leads to sloughing of epithelial cells into the lumen (FIG. 2b-d and FIG. 6a) and ultimately epithelial cell death (FIG. 6b).

e) Restoring Functional AR Via Knock-in of T857A-AR Restores pes-ARKO to a Normal Prostate Phenotype

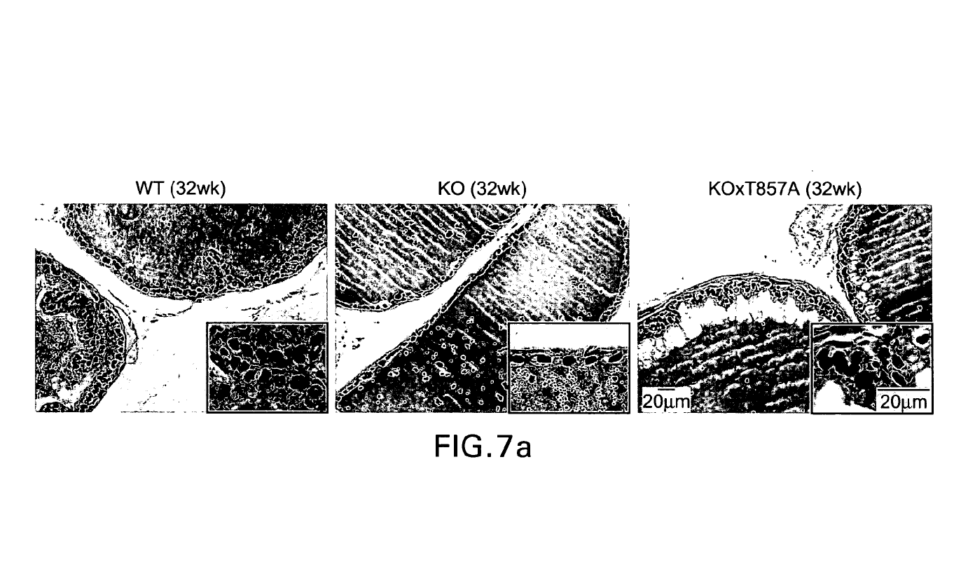

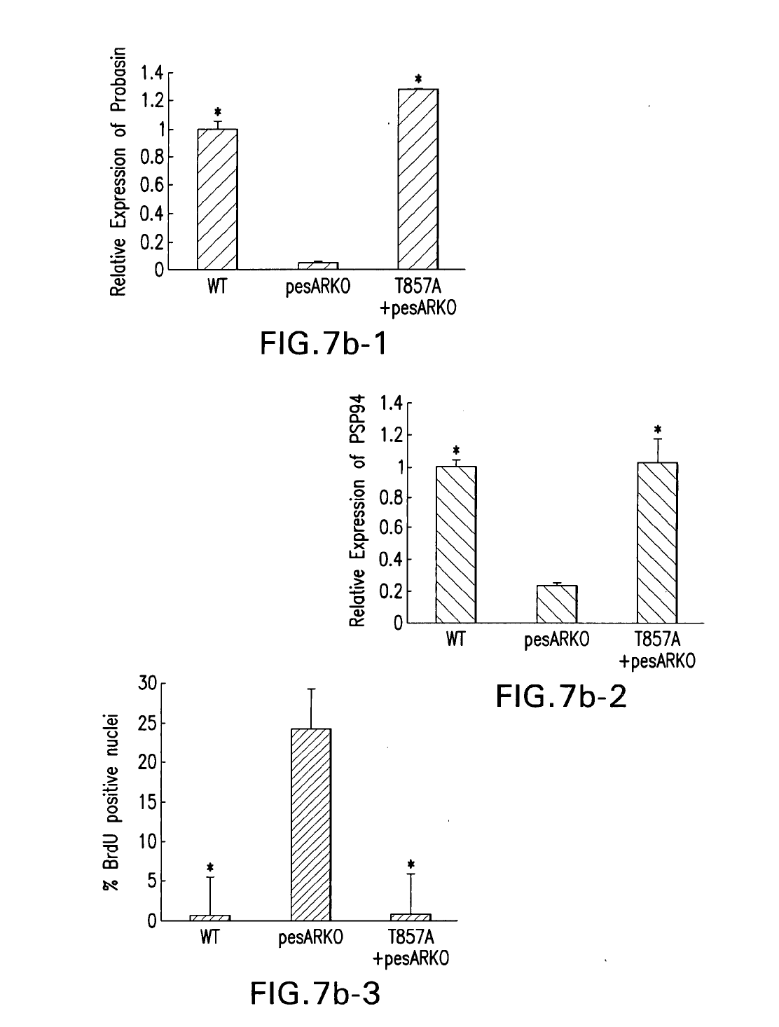

The determination of whether growth and morphological attributes could be rescued after “knock-in” of constitutively activated T857A-AR (mouse AR mutant equivalent to functional human mutant AR, T877A, that is found in LNCaP cells as well as human prostate tumors (Han, G. et al. (2005) Proc Natl Acad Sci USA 102, 1151-6; Han, G. et al. (2001) J Biol Chem 276, 11204-13) into prostate epithelia of pes-ARKO mice was wanted since cellular proliferation and lack of differentiation were observed with removal of WT AR within the prostate epithelia. Therefore, such knock-in mice were created, and then assessed them for restoration of epithelial AR and AR-dependent function. Using the criteria described in the studies above, it was found that restoration of AR to the prostate epithelium restored the AR-dependent effects on the cell proliferation and differentiation (FIG. 7). Collectively, these gain of function via knock-in AR experiments conclusively demonstrate that epithelial AR plays essential roles for cell differentiation and proliferation, a role that has previously been ascribed to stromal factors (Cunha, G. R. & Lung, B. (1978) J Exp Zool 205, 181-93; Cunha, G. R. et al. (2004) J Steroid Biochem Mol Biol 92, 221-36).

To determine whether restoring AR to prostate epithelia of pes-ARKO mice reversed the phenotype pes-ARKO phenotype described, triple mutant mice were created containing T857A-AR, floxAR, and ARRPB2-cre resulting in the T857A/pes-ARKO mice, which have no WT AR within the prostate epithelium, but express transgenic T857A-AR. Genomic DNA (PCR), mRNA (rtPCR), and protein assays (immunohistochemistry) all demonstrated that deletion of WT AR and appropriate expression of T857A-AR in T857A/pes-ARKO mice occurred. No external differences were seen between T857A/pes-ARKO and WT or pes-ARKO mice. Gross dissection of T857A/pes-ARKO mice revealed little differences in internal urogenital organs between WT and pes-ARKO mice at any stage. Importantly, ventral prostates were similar in size compared to WT littermates. Ventral prostates were much smaller in T857A/pes-ARKO mice than in pes-ARKO mice. As anticipated, restoring AR signaling in pes-ARKO mice generated a glandular epithelial phenotype quite similar to that of WT mice at week 32 of age (FIG. 7). This included presence of glandular infolding and tall secretory columnar cells. In addition to restoring normal prostate architecture, the expression of functional AR within the epithelia of T857A/pes-ARKO mice stimulated biochemical changes and re-expression of differentiation markers within epithelium. These changes included increased gene expression of secretory proteins, PSP94 and probasin (FIG. 7). To measure epithelial cell proliferation in T857A/pes-ARKO mice, the BrdU labeling index was determined as described above. Importantly, ventral prostates in T857A/pes-ARKO mice were smaller than those in pes-ARKO mice, indicating a lack of proliferation like that in prostate from WT mice. The restoration of androgen/AR action within pes-ARKO mice significantly reduced epithelial proliferation to levels not different from WT littermates (FIG. 7b, right). Collectively, these gain-of-function experiments show that AR can suppress prostate epithelial proliferation both in situ and in vivo.

f) Conclusion

A key signature of the adult normal prostate gland is the lack of proliferation even in the presence of growth stimulating androgens. This is in contrast to benign prostate hyperplasia and prostate cancer, in which epithelial cells acquire the ability to proliferate. Herein 2 seminal findings in the areas of cell biology and cancer research are disclosed. First it is disclosed that in mature prostatic epithelium, AR is critical for maintaining a differentiated phenotype and overall homeostasis of the gland. Moreover, selective removal of epithelial AR signaling stimulates mitogenesis of the otherwise growth quiescent prostate. These data support the hypothesis that AR in differentiated prostatic epithelium maintains homeostasis via induction of epithelial growth suppressors (or decreased production of growth-stimulatory factors) that can indirectly go through stromal factors or act directly on putative AR-negative stem cells or progenitor cells, thereby inhibiting epithelial proliferation. Secondly, it is disclosed that through four separate means from mice and human prostate cancer cell models that loss of epithelial AR signaling enhances invasiveness and metastatic potential. The mechanisms by which AR mediates these processes may be multi-factorial, involving paracrine factors. Since androgen/AR signaling is aberrant in prostate cancer, it is possible that defects within epithelial AR-induced growth suppressors allow for growth of the stem cells and/or progenitor cells, which in turn facilitate carcinogenesis and ultimately leads to mortality. Normal prostate growth requires a delicate temporal and spatial balance between the proliferative role of stromal AR and the growth-suppressive role of epithelial AR. The findings recast the role of androgen/AR signaling within the prostate, and consequently call into question the current therapeutic strategy for prostate disease, which relies solely and indiscriminately on antagonizing stromal ARs to prevent proliferation without considering epithelial AR's suppressive roles.

g) Methods

(1) Generation of Transgenic Mice

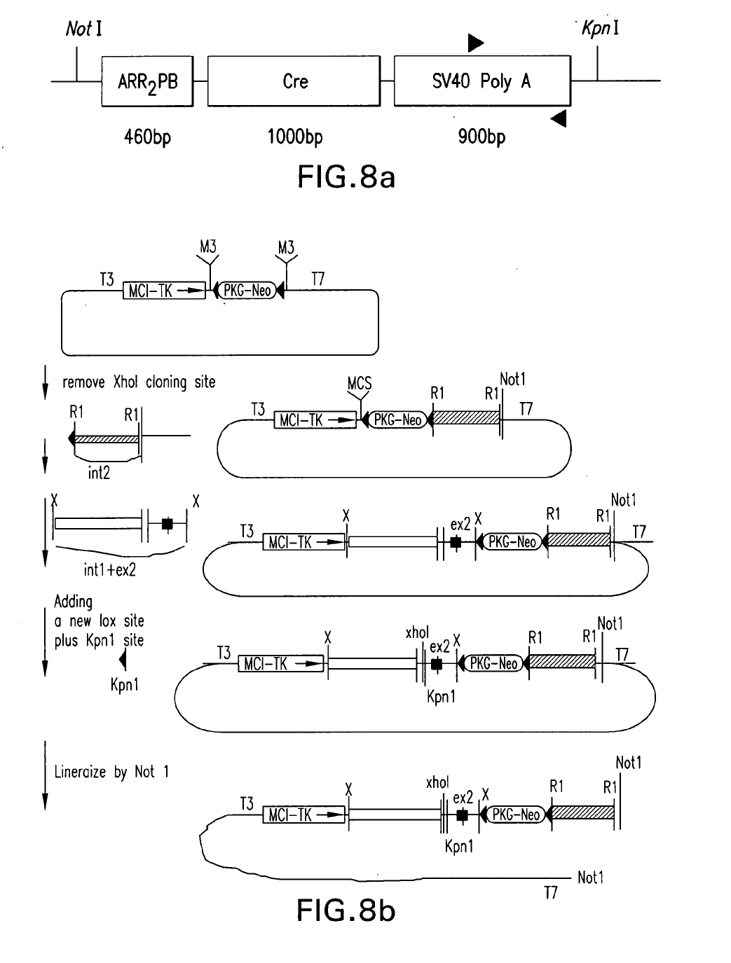

To generate pes-ARKO mice, ARRPB2-Cre transgenic mice (Wu, X. et al. (2001) Mech Dev 101, 61-9) (C57BL/6N, from NIH) were mated with mice (C57BL/6J) containing the conditional AR allele (floxed AR; FIG. 8) (Yeh, S. et al. (2002) Proc Natl Acad Sci USA 99, 13498-503). To generate pes-ARKO/T857A AR mice, the three transgenic mice, ARRPB2-Cre mice (C57BIJ6N), floxed AR mice (C57BL/6j), and T857A AR mice (FVB) (gift from Dr. N. Greenburg, FHCRC, Seattle, Wash.) were interbred.

(2) Statistics

The data was presented as the mean±standard deviation (SD). Comparisons were made between groups using a two-sided Student's t test. P values *P<0.05 or **P<0.01 were considered significant. Survival curves were analyzed by Kaplan-Meier analysis and log-rank tests.

(3) Immunohistochemistry

Pes-ARKO specimens: All three lobes were embedded in the same block and sections prepared 5 um. Immunodetection was performed with the VectaStain kit (Vector Laboratories Inc. Burlingame, Calif.). These antibodies were used: anti-AR(C-19, 1:200), anti-probasin (1:300), anti-E-cadherin (1:100), anti-CK14 (1:50), anti-CK8/18 (1:100), anti-p27 (1:200), anti-RhoB (1:300), anti-PCNA (1:500) (Santa Cruz), anti-p63 (1:50) (abcam), and anti-SMA (1:100) (Sigma). The ratio of AR-positive to total nuclei was calculated in at least 500 cells examined in each of three randomly selected regions.

Immunofluorescence stainings were performed by incubation with primary antibodies (anti-CK8/18, anti-pancytokeratin, anti-calponin) for one hour at room temperature. Sections were then incubated with secondary biotinylated antibodies (Vector Laboratories, Inc. Burlingame, Calif.), followed by FITC or Texas red-conjugated streptavidin (Vector Laboratories, Inc. Burlingame, Calif.). Slides were mounted with antifading medium for microscopic examination.

(4) Pes-ARKO-TRAMP and PC-3 Tissues:

Tissue samples from PLN and liver were fixed overnight in buffered neutral formalin (VWR Scientific Products) at room temperature. The tissues was dehydrated by passing through 70, 85, 95, and 100% ethanol, cleared in xylene and 1:1 xylene/paraffin for 45 min, and embedded in paraffin. The tissue sections were cut at a 5-7-μm thickness for mounting onto Probe-On Plus charged slides (Fisher Scientific). For immunohistochemistry, sections were heated at 55° C. for at least 2 hr, deparaffinized in xylene, rehydrated, and washed in Tris-buffered saline (TBS)/pH 8.0. For antigen retrieval, slides were microwaved in 0.01 M sodium citrate/pH 6.0, immersed with 1% hydrogen peroxide in methanol for 30 min, and blocked with 20% normal goat serum in TBS for 60 min. After washing with PBS, sections were incubated for 90 min with T-ag, AR and pan-CK-10 antibodies diluted 1:200 in TBS containing 1% BSA, followed by goat anti-rabbit biotinylated secondary antibody diluted 1:300 in TBS containing 1% BSA. Slides were counterstained with hematoxylin for 30 sec, dehydrated, cleaned in xylene, and mounted and replaced primary antibody with normal rabbit IgG or 1% BSA in TBS for negative controls.

(5) RNA Isolation and Analysis

Total cellular RNA was isolated from each lobe and used to synthesize random primed first strand complementary DNA for analysis by RT-PCR or real time PCR. Amplification of AR exon 2, Nkx3.1, probasin, and PSP 94 (see Table 5 for primer sequence) were normalized to beta-actin in each sample.

TABLE 5
Sequence for primers used in RT-PCR

| Gene | Sequence |
|---|---|
| Probasin | F: 5′-ATC ATC CTT CTG CTC ACA CTG CAT G-3′ |
| | SEQ ID NO: 14 |
| | R: 5′-ACA GTT GTC CGT GTC CAT GAT ACG C-3′ |
| | SEQ ID NO: 15 |
| PSP-94 | F: 5′-CCT GTA AGG AGT CCT GCT TTG TC-3′ |
| | SEQ ID NO: 16 |
| | R: 5′-ATG CTG GCT CTG CCT TCT GAG T-3′ |
| | SEQ ID NO: 17 |
| Nkx3.1 | F: 5′-AGA CAC GCA CTG AAC CCG AGT CTG ATG |
| | CAC-3′ |
| | SEQ ID NO: 18 |
| | R: 5′-AGA CAG TAC AGG TAG GGG TAG TAG GGA |
| | TAG C-3′ |
| | SEQ ID NO: 19 |

(6) Apoptosis Assay

The In Situ Cell Death Detection Kit (Roche Pharmaceuticals, Nutley, N.J.) was used according to the manufacturer's instructions for detection of apoptotic cells.

(7) BrdU Labeling Indices

Mice were injected with BrdU (30 ug/gm body weight, Sigma) I.P. 24 hours before sacrificed. The BrdU-labeled epithelial cells were detected employing a monoclonal anti-BrdU antibody (Zymed Laboratories, San Francisco, Calif.) according to manufacturer's direction. The labeled cells were calculated from multiple fields of each slide. Several sections from each prostate were analyzed to obtain the mean of BrdU positive epithelial cells. The means of the proliferating cells from each lobe of prostate were reported.

### 2. Example 2

### Prostate Epithelial Androgen Receptor Functions as Suppressor for the Prostate Cancer Metastasis

Experimental evidence from study of anaplastic prostate cancer cell lines has led to the idea that epithelial AR, when activated by androgen, increases cellular proliferation (Bello, D., et al. (1997) Carcinogenesis 18, 1215-1223; Danielpour, D., et al. (1994) Cancer Res. 54, 3413-3421; Suzuki, H., et al. (2003) Endocr Relat Cancer 10, 209-16) Clinical studies also point out that androgen deprivation therapy (ADT) with suppression of androgen/androgen receptor (AR) functions, is an effective treatment for most prostate cancer patients ((1967) Surg. Gynecol. Obstet. 124:1011-1017; Messing, E. M., et al. (1999) N. Engl. J. Med. 341, 1781-1788). However, most patients' tumors re-grow after 1-2 years of continuous ADT (Eisenberger, M. A., et al. (1998) New Engl. J. Med. 339, 1036-1042). The detailed mechanisms of why suppression of androgens/AR ultimately fails to suppress prostate tumor metastasis remain unclear.

Herein it is disclosed that the generation of the first mouse (pes-ARKO-TRAMP) that generates prostate cancer spontaneously which lack AR only in prostate epithelia. Notably, it is demonstrated, through AR gain- and loss-of-function and co-culture with epithelium-stroma cell experiments, novel suppressive roles of epithelial AR within the prostate cancer cells. The counter-intuitive ideas brought forth from herein revolutionizes the way prostatic disease is combated.

a) Results

(1) Epithelial AR is a Suppressor and Stromal AR is a Stimulator for the Prostate Cancer Cell Invasion.

(a) PC3-v Cells Vs PC3-AR9 Cells

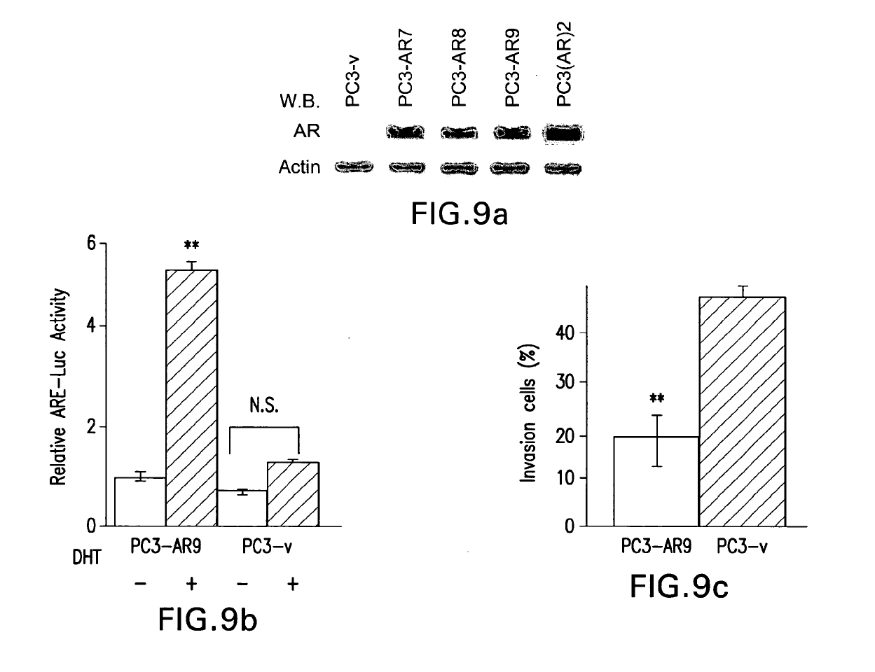

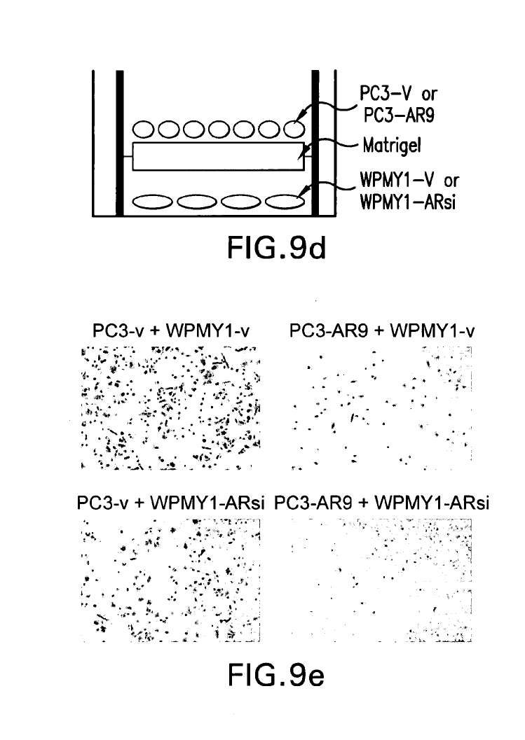

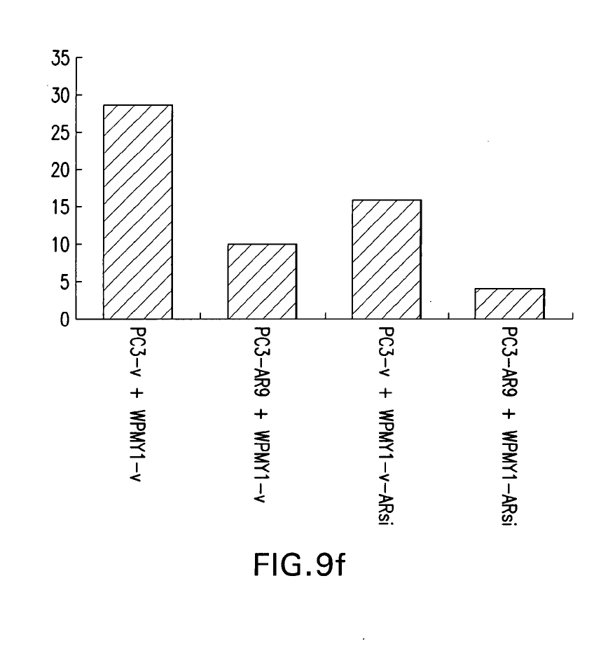

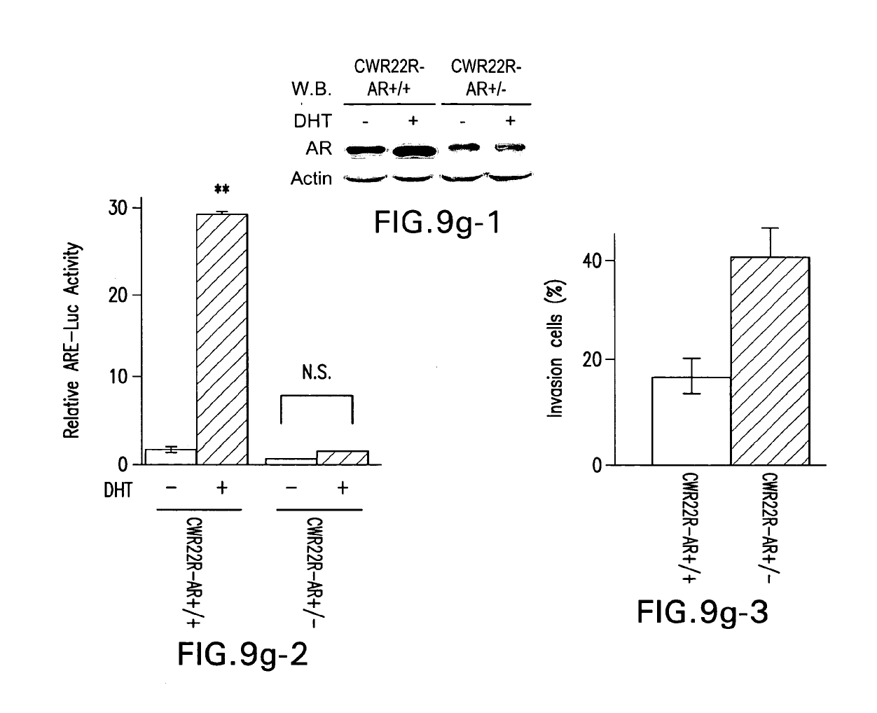

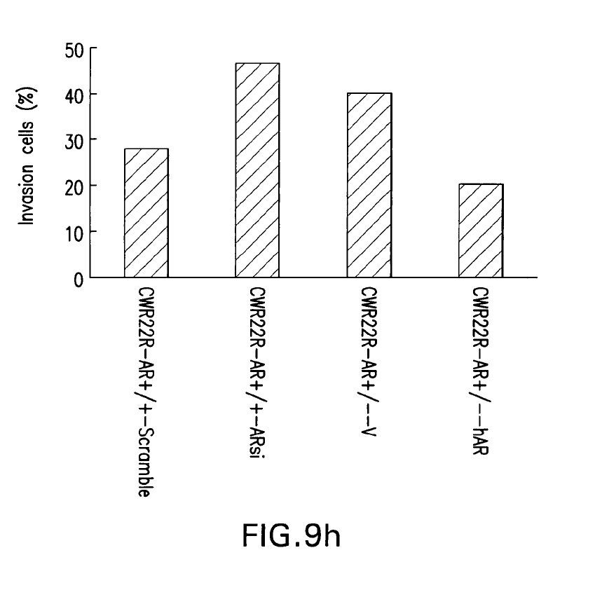

To dissect how AR influences prostate cancer metastasis, prostate cancer PC3 cells that were originally isolated from a bone metastatic tumor from a prostate cancer patient were stably transfected with a functional AR cDNA linked with a human AR promoter (Mizokami, A., et al. (1994) Mol. Endocrinol. 8, 77-88). Unlike other cell models where AR is over-expressed with strong viral promoters (Yuan, S. et al. (1993) Cancer Res. 53, 1304-1311) and leads to an unnatural build up of AR, these cells, designated PC3-AR9 express a normal amount of functional AR and are activated by the androgen dihydrotestosterone (DHT) (FIG. 9a-b). Comparing the two lines using the invasion assay, PC3-AR9 cells were found to be significantly less invasive than the parental PC3 cells that stably transfected with vector only (named as PC3-v) (FIG. 9c).

(b) WPMY1-v Cells Vs WPMY1-ARsi Cells

These surprising results, when contrasted against the classic concept in the prostate field that believes the prostate AR should function as stimulator to promote prostate cancer progression, encouraged the further application of co-culturing PC3-v cells with stromal WPMY1 (Webb, M. M. et al. (1999) Carcinogenesis 20, 1185-1192) cells to verify the AR role in prostate cancer cell invasion. Early reports demonstrated that functional AR expressed in the WPMY1 cells led to promotion of androgen-dependent keratinocyte growth factor (KGF) gene expression Heitzer, M. D. & DeFranco, D. B. (2006) Cancer Res. 66, 7326-7333). Herein, the endogenous AR expression was knockdowned in WPMY1 cells with stable transfection of AR-siRNA (named as WPMY1-ARsi) and co-cultured with PC3-v cells on the different layer of the Boyden chamber (FIG. 9d) for the cell invasion assay. The result indicated that knockdown of stromal AR in WPMY1-ARsi cells result in the suppression of epithelial PC3-v cell invasion (FIG. 9e-f). In contrast, result from co-culture of WPMY1-v cells that transfected vector only with either PC3-v or PC3-AR9 cells in Boyden chamber indicated that addition of epithelial AR in PC3-AR9 cells result in the suppression of epithelial cell invasion (FIG. 9e-f), and co-culture of PC3-AR9 cells with WPMY1-ARsi cells further suppressed cell invasion (FIG. 9e-f). These co-culture results confirmed FIG. 9c and indicates that the epithelial AR functions as suppressor for prostate cancer cell invasion and stromal AR functions as promoter to stimulate prostate cancer cell invasion.

(c) CWR22R-AR^{+/+} Cells Vs CWR22R-AR^{+/-} Cells

To further confirm the unexpected results showing epithelial AR functions as suppressor for prostate cancer cell invasion, another approach using homologous gene recombination strategy (Yeh, S. et al. (2003) J. Exp. Med. 198, 1899-1908) was applied to knockdown AR in human CWR22R prostate cancer cells isolated from hormone-refractory prostate tumor (Nagabhushan, M., et al. (1996) Cancer Res. 56, 3042-3046). As shown in FIG. 9g, CWR2R-AR^{+/-} cells expressed much less AR with negligible AR transactivation as compared to the parent CWR22R-AR^{+/+} cells. Boyden chamber invasion assay again demonstrated that knockdown AR in CWR22R-AR^{+/-} cells result in more invasive as compared to parent CWR2R-AR^{+/+} cells (FIG. 9g, low panel). Using different approach via AR-siRNA to knockdown AR in CWR22R-AR^{+/-} cells also result in the similar conclusion with more invasive as compared to parent CWR22R-AR^{+/+} cells and addition of functional AR back to CWR22R-AR^{+/-} cells result in decreased cell invasion as compared to CWR22R-AR^{+/-} cells transfected empty vector only (FIG. 9h).

Together, results from FIG. 9 using four different approaches (knock-in, knockdown with genetic recombination, knockdown with siRNA and co-culture system) with different prostate cancer cells all demonstrated that epithelial AR function as suppressor to suppress the prostate cancer invasion.

(2) Addition of Functional AR in PC3-AR9 Cells Result in Decreased Invasion in In Vivo Mice Models.

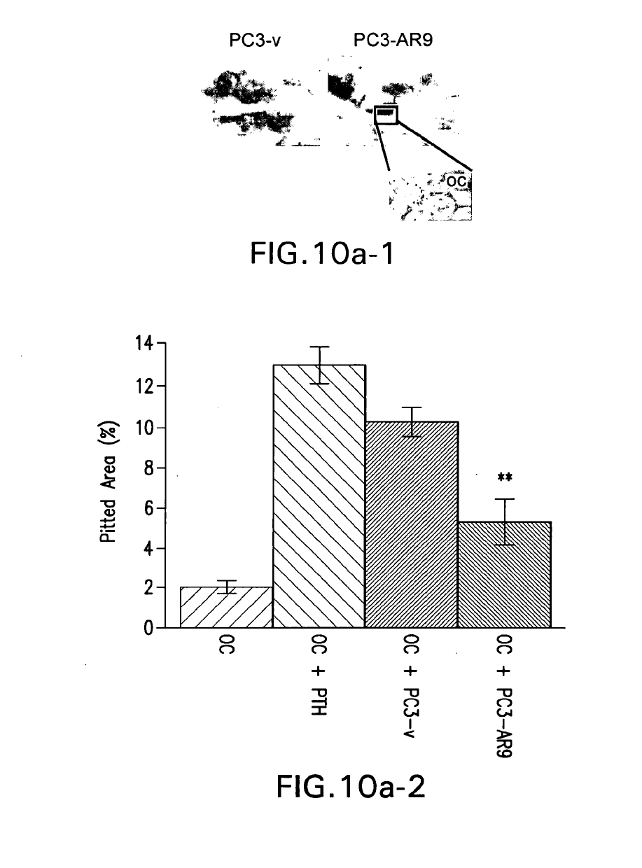

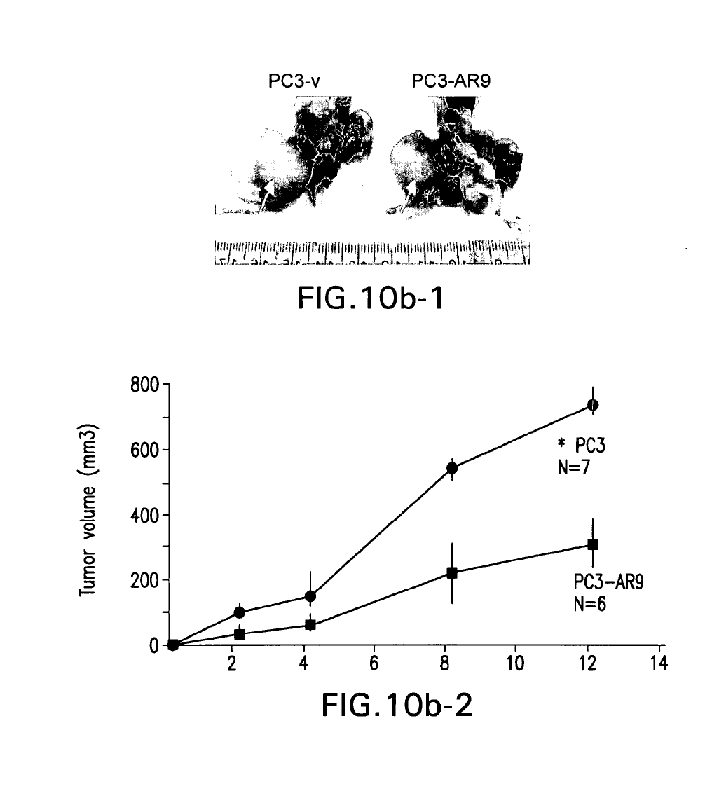

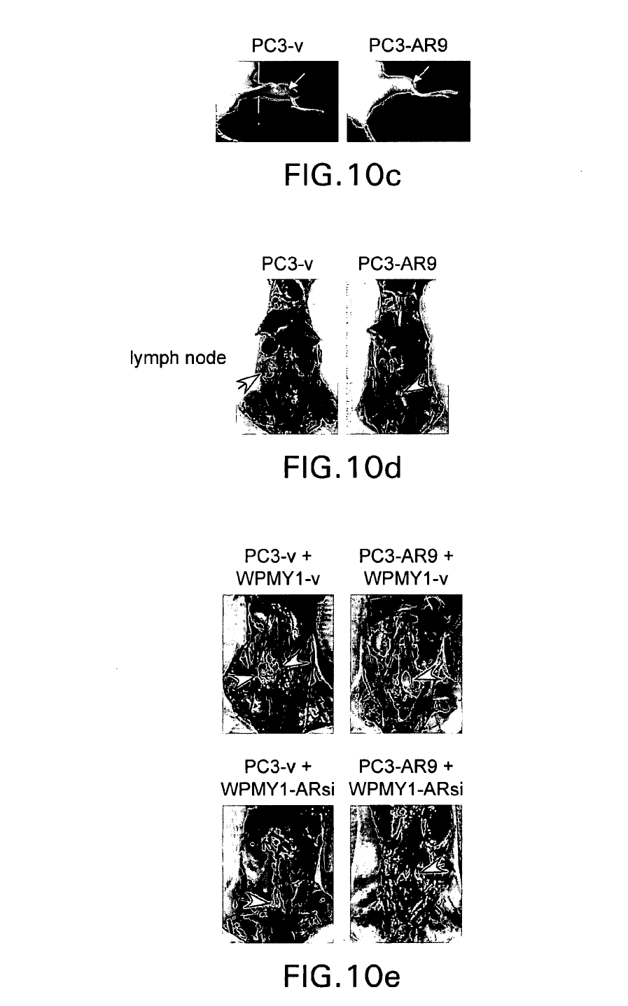

As PC3 cells were isolated from bone metastatic tumor, the invasive ability of AR in PC3-AR9 cells contacted with bone was further examined. Osteoclastogenesis was assayed in a bone-wafer resorption assay (Goater, J. J., et al. (2002) J. Orthop. Res. 20, 169-173). PC3-v or PC3-AR9 cells were co-cultured with bone cells from newborn rat bone marrow layered onto bone wafers, and osteoclast formation (Goater, J. J., et al. (2002) J. Orthop. Res. 20, 169-173) was quantified. Results from this experiment demonstrated a decreased number of osteoclytic lesions (pitted areas) in PC3-AR9 cells as compared to parental PC3-v cells (FIG. 10a). To evaluate invasion characteristics in vivo, cells were injected into the tibia of nude mice (Corey, E. et al. (2002) Prostate 52, 20-33). PC3-v tumors grew more aggressively (FIG. 10b) and more invasively (FIG. 10c) than PC3-AR9 as determined by x-ray analysis. Collectively, these gain-of-function data from knock-in of functional human AR show that loss of the prostate epithelial AR signaling directly promotes invasion and indirectly affects the surrounding micro-environment, both in vitro and in vivo.

Furthermore, the AR roles in metastatic assays were tested with in vivo mice model. Either PC3-v or PC3-AR9 cells were directly injected orthotopically into anterior prostate of nude mice. As expected, mice with injected PC3-v cells developed bigger metastatic tumor in the lymph node as compared to those from injected PC3-AR9 cells (FIG. 10d).

Those different combinations of co-cultured PC3-v, PC3-AR9, WPMY1-v and WPMY1-ARsi were also injected orthotopically into the anterior prostate of nude mice. As expected, these in vivo results are consistent with in vitro data showed in FIG. 9 that addition of epithelial AR in PC3-AR9 developed less metastatic tumor in lymph node and knockdown AR in stromal WPMF1-ARsi cells also developed less metastatic tumor in lymph node (FIG. 10e).

Together, using either knockdown or knock-in AR in various human prostate cancer cells, the in vitro cell line data and in vivo mice data all demonstrate that epithelial AR functions as suppressor to suppress prostate metastatic tumor invasion and stromal AR functions as stimulator to promote prostate metastatic tumor invasion.

(3) Pes-ARKO-TRAMP Mice Develop more Aggressive and Invasive Metastatic Tumors

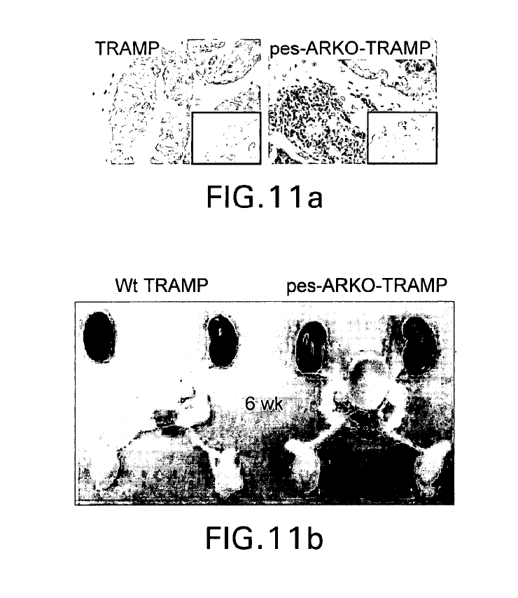

So far, all above data, either from in vitro cell co-culture system or in vivo mice models, were all generated from human prostate cancer cells. Thus the experiments were modified to use mice that can spontaneously developed prostate tumor as another in vivo animal model to prove the above conclusion. Female flox/AR(C57BL/6/128) mice (Suzuki, H., et al. (2003) Endocr Relat Cancer 10, 209-16) were mated with TRAMP (C57BL/6/TRAMP×FVB) mice (Hill, R. E., et al (2003) Exp. Biol. Med. 228, 818-822) to generate flox/AR-TRAMP(C57BL/16/129×TRAMP-FVB) mice, these mice were crossed with Pb-Cre (C57BL/6) mice (Wu, X., et al. (2001) Mech. Dev. 101, 61-69) to generate pes-ARKO-TRAMP(C57BL/6/129×TRAMP-FVB) mice that lack the AR only in prostate epithelium. Immunohistochemical analysis of prostate tumors using the C-19 antibody specific to the AR C-terminal region (Mirosevich, J. et al. (1999) J. Endocrinol. 162, 341-350) showed that only the tumors of Wt-TRAMP mice expressed the AR (FIG. 11a, left panel), whereas the tumors of pes-ARKO-TRAMP mice expressed little AR (FIG. 11a, right panel). As expected, the urogenital organs from both Wt-TRAMP and pes-ARKO-TRAMP mice develop normally (FIG. 11b).

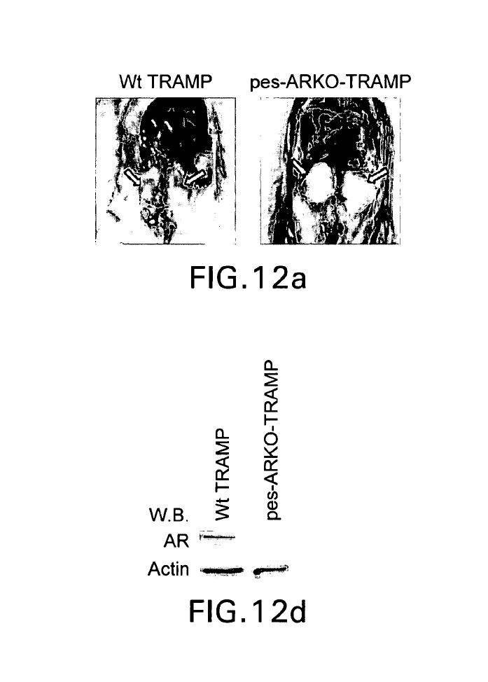

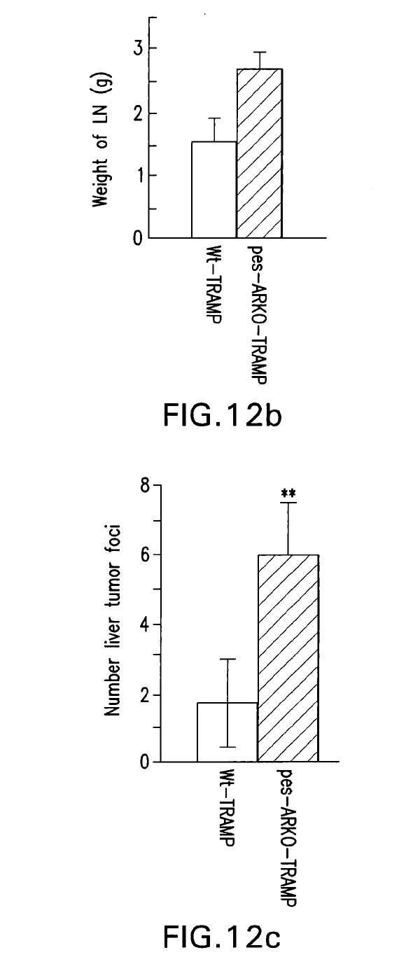

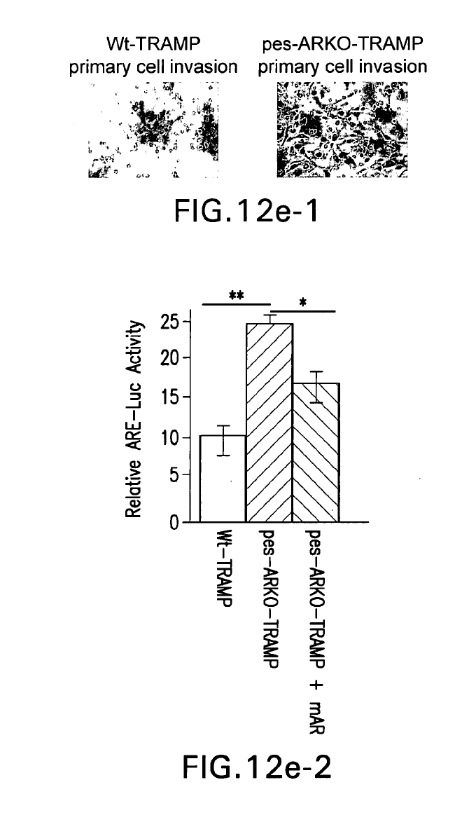

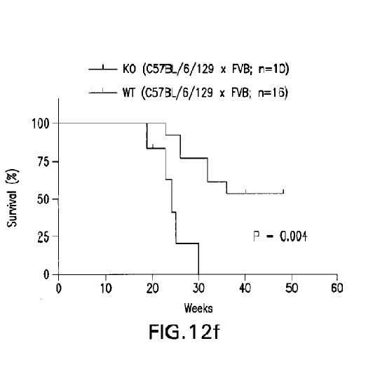

It was found that pes-ARKO-TRAMP mice had significantly larger (P<0.05; 3.0 vs 1.7 mg) pelvic lymph nodes (PLN) than WT-TRAMP mice (FIG. 12a-12b). Moreover, more prostate metastatic foci were observed within the liver of pes-ARKO-TRAMP mice (FIG. 12c). Western analysis confirmed loss of AR within PLN from pes-ARKO-TRAMP mice (FIG. 12d). To evaluate invasiveness of TRAMP cells, the Boyden chamber matrigel invasion assay (Pilatus, U. et al., (2000) Neoplasia 2, 273-279) was used on primary cultured PLN cells from both strains of mice. PLN cells from pes-ARKO-TRAMP mice were more invasive than those from WT-TRAMP mice (FIG. 12e). Moreover, restoring functional AR reduced the invasiveness of PLN primary tumor cells (FIG. 12e). Together, results from pes-ARKO-TRAMP mice (FIG. 12a-d) and primary culture cells from PLN tumor studies (FIG. 12e) show that loss of prostate epithelial AR leads to the development of more invasive metastatic prostate tumors and gain of AR function reverses the increased invasion in PLN cells from pes-ARKO-TRAMP mice. The more invasive metastatic prostate tumors then lead to lower survival rates in pes-ARKO-TRAMP mice as compared to those from WT-TRAMP littermates (FIG. 12f).

(4) Ind-ARKO-TRAMP Mice Develop Less Aggressive and Invasive Metastatic Tumors.

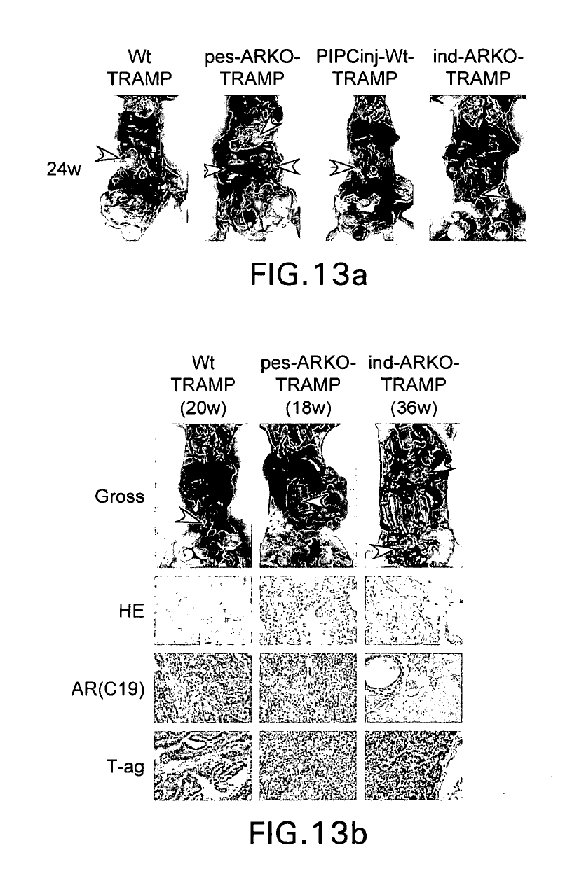

An inducible knockout system was also used to generate Ind-ARKO-TRAMP mice that knockdown both prostate epithelial AR (˜50%-60%) and stromal AR (˜50%) via injection of interferon v (PIPC)(Kuhn, R. et al. (1995) Science 269, 1427-1429) into 12 wks of ind-ARKO-TRAMP mice. A comparison of metastatic tumor size among 24 wks of pes-ARKO-TRAMP mice, wild type mice (with or without injection of PIPC at 12 weeks) and ind-ARKO-TRAMP mice that injected with PIPC at 12 weeks, revealed that pes-ARKO-TRAMP mice develop bigger and more aggressive metastatic tumor in lymph nodes as compared to their wild type littermates (FIG. 13a). In contrast, knockdown AR in both epithelial and stromal cells in ind-ARKO-TRAMP mice led to development of smaller and less aggressive metastatic tumor in lymph nodes as compared to their wild type littermates injected with IPIC (FIG. 13a).

Since prostate tumor developed at different rates between pes-ARKO-TRAMP mice and ind-ARKO-TRAMP mice, another approach was taken to compare the development of metastatic tumors between these two different mice. It was found that pes-ARKO-TRAMP mice need 18 wks to develop prostate primary tumor with size near 1 cm diameter. However, it took 36 wks for ind-ARKO-TRAMP to develop the similar size of prostate primary tumor (FIG. 13b). Moreover, different degree of malignancy was found in these two tumors with similar size with more aggressive tumor found in pes-ARKO-TRAMP mice. Furthermore, pes-ARKO-TRAMP mice tumor migrated into mesentery lymph nodes and ind-ARKO-TRAMP mice migrated into seminal vesicle at the stage when primary tumors develop to 1 cm diameter (FIG. 13b).

Together, results from FIG. 13 clearly demonstrate that knockdown AR in both epithelial and stromal cells leads to the development of the smaller and less aggressive metastatic tumor in lymph nodes as compared to their wild type littermates. In contrast, loss of epithelial AR results in more aggressive metastatic tumor as compared to their wild type littermates as well as those loss of both epithelial and stromal AR. Therefore, stromal AR that functions as stimulator plays more dominant roles as compared to epithelial AR that functions as suppressor of prostate metastasis.

(5) AR Expression is Decreased in Metastases as Compared with Primary Prostate Tumor Isolated from Prostate Cancer Patients.

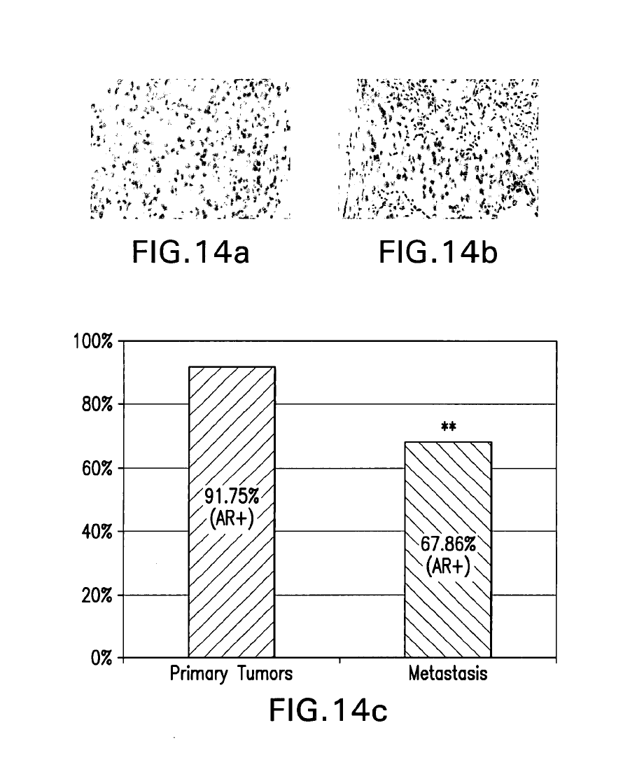

Direct clinical data survey via assay of intensity of AR staining in prostate metastases and primary tumors from prostate cancer patients also supported the above conclusion that epithelial AR functions as suppressor for prostate metastasis. Prostate primary tumors (97 cases) and prostate metastases (28 cases), were evaluated and AR nuclear staining was found in all AR-positive tumors and a significant difference was found in AR expression between primary tumors (91.75%) and metastatic tumors (67.86%), (P<0.01) (FIG. 14). These in vivo clinical data are consistent with a recent clinical study (Li, R. et al. (2004) Am. J. Surg. Pathol. 28, 928-934) which utilized tissue arrays from prostate cancer patients treated with radical prostatectomy where it was concluded that AR expression was significantly decreased in metastatic prostate cancer as compared to primary prostate cancer or normal prostate (mean 1.30 vs 3.49, P=0.000). The fact that AR expression decreased in metastatic tumors as compared to primary tumors in these two in vivo clinical studies supports the negative role of AR in prostate metastatic tumors.

(6) Impact to Current Clinical Treatment to Prostate Cancer

As results from different human prostate tumor cells (FIGS. 9, 10) and various prostate tumor mice models (FIG. 10-13), as well as human clinical data (FIG. 14) all pointed out that epithelial AR functions as suppressor and stromal AR functions as stimulator for the prostate metastasis, it was investigated whether these results which are surprising and contradictory to the classic concept of prostate AR roles (shall not function as suppressor) influence the current prostate cancer therapy. Based on above conclusions, the ideal therapeutic approach to battle the prostate cancer is to target stromal AR, either via AR-siRNA or compound, such as ASC-J9 to suppress or degrade AR in stromal cells only. Unfortunately, so far no such ideal stromal-specific deliver system can send those AR-killers to target stromal AR only. Furthermore, all current surgical or chemical castration with available antiandrogens is target mainly to androgens and not AR (that can be the reason why patients with hormone refractory prostate tumor after antiandrogens treatment still maintain relative higher amount of AR in prostate tumor (Chen, C. D., et al. (2004) Nat. Med. 10, 33-39)). Nevertheless, even when only whole AR can be targeted as was done in the ind-ARKO-TRAMP mice, prostate cancer can still be battled effectively with right timing. Based on the ind-ARKO-TRAMP mice model that target AR at different period, early targeting of AR via knockdown AR (at 4 weeks) results in a much better suppression of prostate tumor as compared to those did at later time (at 20 weeks) (see Table 1). These results indicate that not only stromal AR plays more dominant roles than epithelial AR, it also points out that targeting AR at earlier stage can be a better strategy to battle prostate cancer.

TABLE 1
Table 1. AR knockout induced on ind-ARKO-TRAMP mice at early
age (4 ws) significantly reduced tumor genosis and progression, while
at later age of 20 ws failed to block tumor progression.

| | | | | | induced ARKO | |
|---|---|---|---|---|---|---|
| | | | induced ARKO | | at 20 ws | |
| | Wt TRAMP | | at 4 ws | | lymph | |
| age | tumor | lymph node | tumor | lymph node | tumor | node |

20 ws

| | | | | |
|---|---|---|---|---|
| 1. | 3 mm | — | — | — |
| 2. | 3 mm | — | — | — |
| 3. | 5 mm | — | — | — |
| 4. | 7 mm | — | — | — |
| 5. | 15 mm | 4 mm | — | — |

24 ws

| | | | | | | |
|---|---|---|---|---|---|---|
| 1. | 15 mm | 3 mm | — | — | 9 mm | — |
| 2. | 18 mm | — | — | — | 16 mm | 3 mm |
| 3. | 20 mm | 10 mm | — | — | 19 mm | 2 mm |
| 4. | 25 mm | 5 mm | 5 mm | — | 20 mm | 5 mm |

| | | | | | |
|---|---|---|---|---|---|
| 5. | 25 mm | 5 mm | Die without tumor | 25 mm | 10 mm |

28 ws

| | | | | | | |
|---|---|---|---|---|---|---|
| 1. | 20 mm | 5 mm | — | — | 21 mm | 3 mm |
| 2. | 30 mm | 7 mm | — | — | 22 mm | 5 mm |
| 3. | 30 mm | 5 mm | — | — | 25 mm | 11 mm |

| | | | | |
|---|---|---|---|---|
| 3. | Die of tumor | 5 mm | 3 mm | Die of tumor |
| 4. | Die of tumor | 15 mm | 7 mm | Die of tumor |

32 ws

| | | | | | | |
|---|---|---|---|---|---|---|
| 1. | 24 mm | 10 mm | — | — | 25 mm | 10 mm |

| | | | | | |
|---|---|---|---|---|---|
| 2. | Die of tumor | — | — | 28 mm | 5 mm |

| | | | | |
|---|---|---|---|---|
| 3. | Die of tumor | — | — | Die of tumor |
| 4. | Die of tumor | 3 mm | — | Die of tumor |
| 5. | Die of tumor | 6 mm | 3 mm | Die of tumor |

(7) Molecular Mechanisms by which Epithelial AR Promotes Prostate Metastatic Tumor Invasion

To dissect the molecular mechanisms by which AR promotes prostate metastatic tumor invasion, Q-PCR was used to quantify mRNA expression of most reported prostate metastasis/invasion-related genes whose expression closely correlates with prostate tumor metastasis. As shown in Table 2, the relative mRNA expression of most metastasis/invasion-related genes in PLN tumors from WT-TRAMP and pes-ARKO-TRAMP mice as well as xenografted tumors from PC-3 vs PC3-AR9 correlate well with their metastatic status.

TABLE 2
Table 2. Expression profiles of prostate metastasis/invasion related
genes in pelvic lymph node tumor (PLN) of pes-ARKO-TRAMP mice
and PC3 xenograft tumors compared to PLN of Wt TRAMP mice and
PC3-AR3 xenograft tumors, respectively.

| | pes-ARKO- | | | PC3 | | |
|---|---|---|---|---|---|---|
| Genes | TRAMP(PLN) | | | (Xenograft tumors) | | |
| NEP | −5.9 | ↓ ** | ±0.172 | −3.7 | ↓ ** | ±0.282 |
| Cox-2 | 3.3 | ⇑ ** | ±0.212 | 6.2 | ⇑ ** | ±0.141 |
| P27 | −4.6 | ↓ ** | ±0.327 | −2.5 | ↓ ** | ±0.353 |
| MMP-2 | 1.2 | N.S. | ±0.244 | 0.54 | N.S. | ±0.494 |
| MMP-9 | 6.8 | ⇑ * | ±0.172 | 8.5 | ⇑ ** | ±0.211 |
| EGF-R | 4.7 | ⇑ * | ±0.172 | 3.7 | ⇑ ** | ±0.582 |
| IGF-2 | 3.5 | ⇑ * | ±0.333 | 8.1 | ⇑ ** | ±0.228 |
| IL-6 | 3.5 | ⇑ ** | ±0.070 | 3.9 | ⇑ ** | ±0.499 |
| TNF | 3.6 | ⇑ * | ±0.472 | 4.5 | ⇑ ** | ±0.282 |
| MEN1 | 5.2 | ⇑ ** | ±0.272 | 4.5 | ⇑ ** | ±0.707 |
| KRIP | −2.9 | ↓ ** | ±0.472 | −2.6 | ↓ ** | ±0.482 |

Quantitative RT-PCR data represent fold increase over PLN tumor Wt TRAMP mice or PC3-AR9 xenograft tumors.
Statistic analysis mean ±SD
*P < 0.05,
**P < 0.01,
N.S. indicated no significant difference.

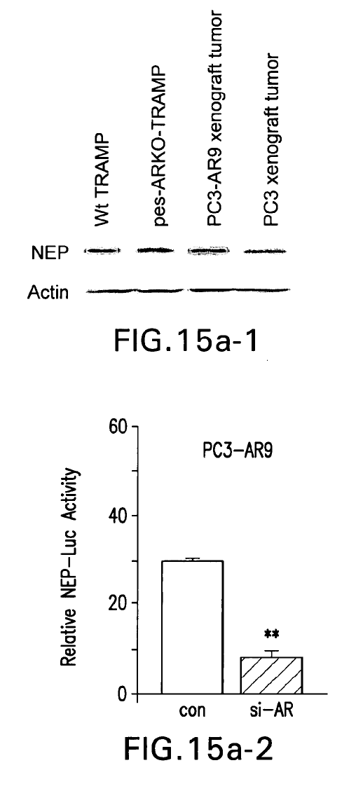

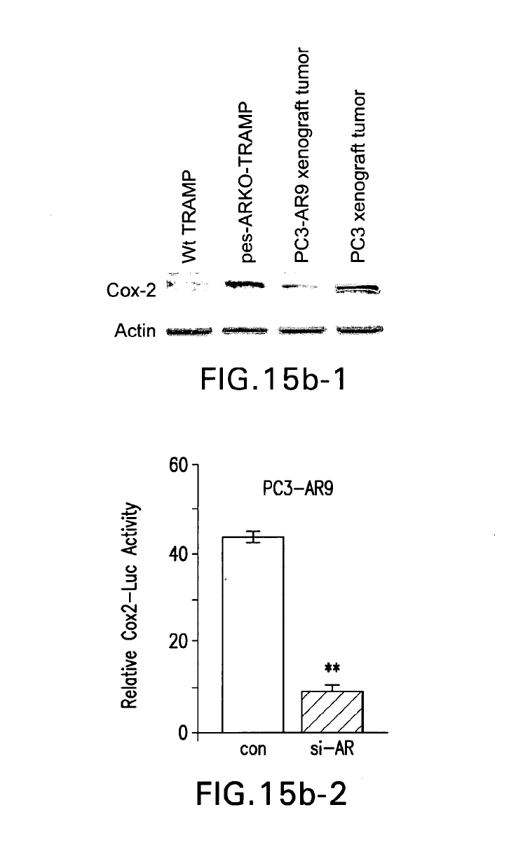

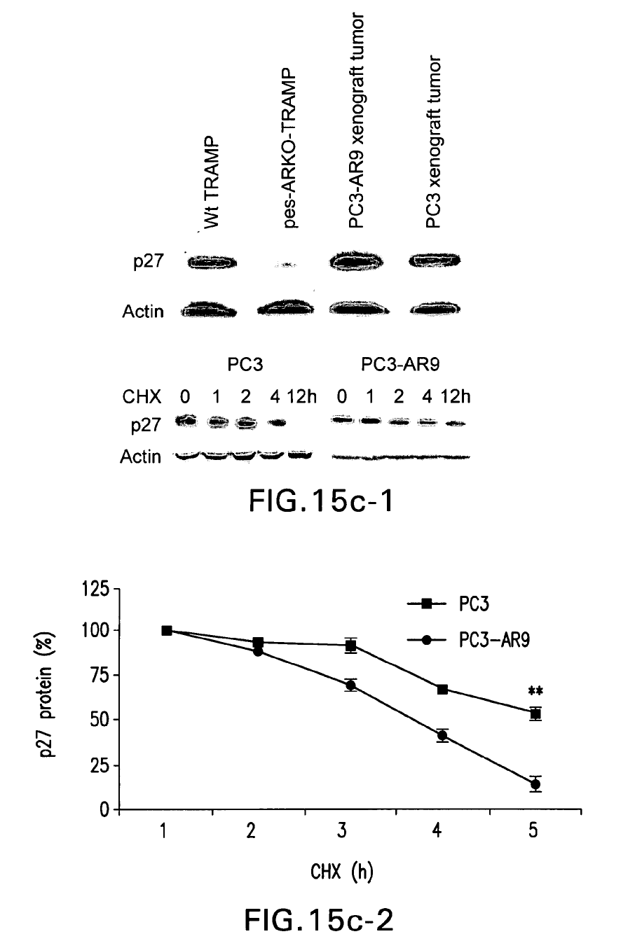

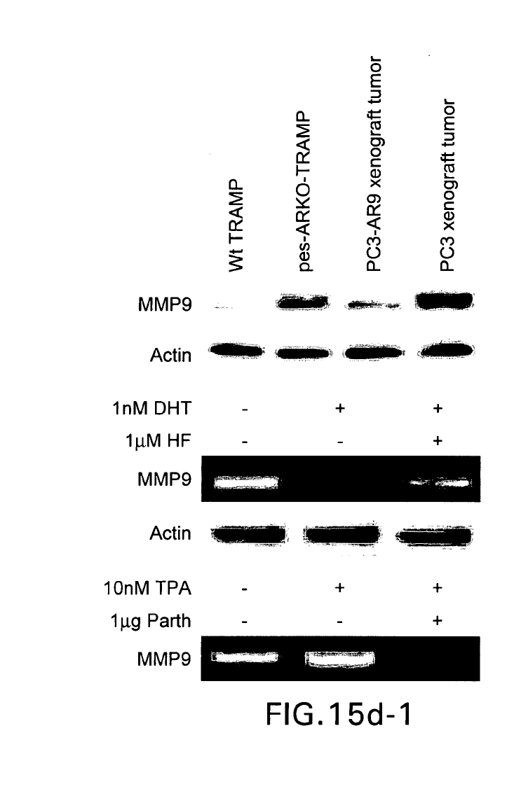

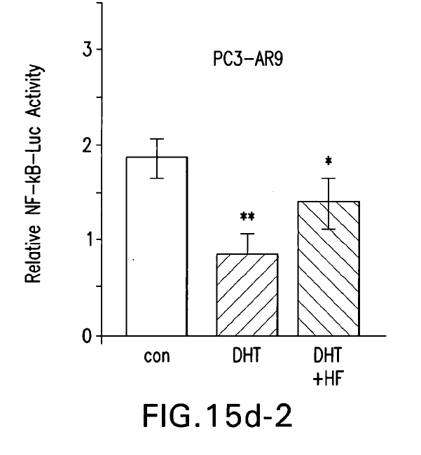

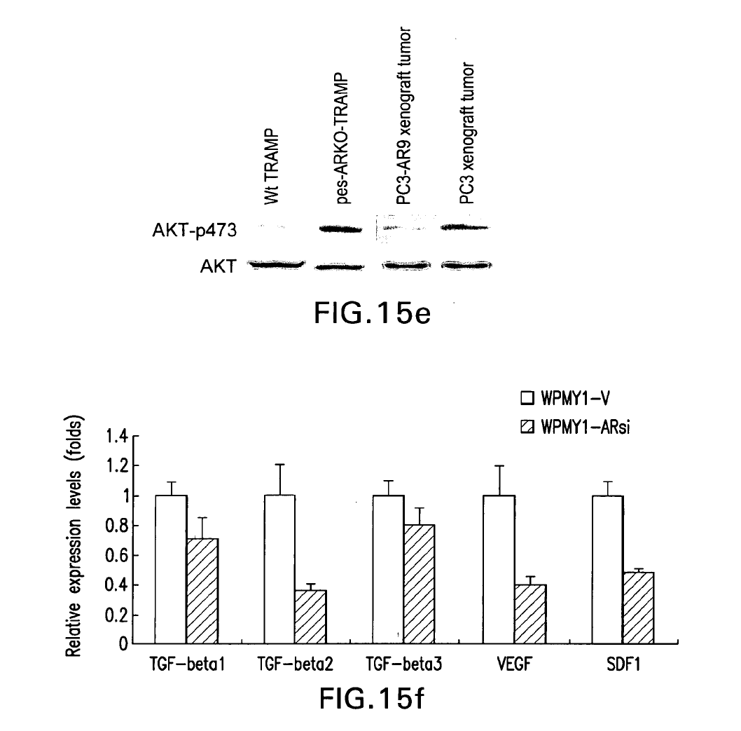

These results strengthen the in vivo findings showing that loss of the prostate epithelial AR promotes prostate metastatic tumor invasion. Further mechanistic dissection, which was presented in FIG. 15, indicates that the AR utilizes multiple mechanisms to modulate those metastasis/invasion-related genes.

(8) Molecular Mechanisms by which Epithelial AR Suppresses Prostate Metastatic Tumor Invasion

Further mechanism dissection indicates that the AR utilizes multiple mechanisms to modulate those metastasis/invasion-related genes.

(a) Androgen/AR Modulates Expression of Some Metastasis/Invasion-Related Genes, Such as Neural Endopeptidase (NEP) and Cyclooxygenase 2 (Cox-2) at the Transcriptional Level.

NEP is capable of degrading neuropeptides, such as bombesin, endothein-1, and neurotensin that have been implicated in promoting the prostate cancer cell migration (Nelson, J. B. & Carducci, M. A. (2000) Cancer Invest. 18, 87-96; Papandreou, C. N. et al. (1998) Nat. Med. 4, 50-57; Sehgal, I. et al. (1994) Proc. Natl. Acad. Sci. USA 91, 4673-4677). Herein, NEP mRNA (Table 1) and protein (FIG. 15a) expression are lower in both metastatic tumors lacking AR. Transactivation using a NEP 5′ promoter-Luciferase assay further confirms that the induced NEP transcription is suppressed by adding the AR-siRNA (FIG. 15a, lower panel).

It has been shown that the expression of Cox-2 is elevated in prostate cancer (Saunders, M. A., et al. (2001) J. Biol. Chem. 276, 18897-18904), and inhibition of Cox-2 activity in prostate tumors suppresses their invasion (Attiga, F. A., et al. (2000) Cancer Res. 60, 4629-4637). Herein, Cox-2 mRNA (Table 1) and protein (FIG. 15b upper panel) expression are higher in both metastatic tumors lacking the AR. It was also shown herein that transactivation using Cox-2 5′ promoter-Luciferase assays that Cox-2 transcription was suppressed by addition of more functional AR via pBabe virus expressed AR cDNA into PC3-AR9 cells (FIG. 15b lower panel).

(b) Androgen/AR Enhances the Protein Levels of Metastasis/Invasion-Related Genes, Such as p27.

p27 is a cyclin-dependent kinase inhibitor and down-regulation of p27 has been linked to local invasion or distant metastasis in many tumors (Belletti, B. et al. (2005) Curr. Med. Chem. 12, 1589-1605). Recently, it has been reported that cytoplasmic p27 protein inhibits tumor migration and invasion (Baldassarre, G. et al. (2005) Cancer Cell 7, 51-63). Herein, both p27 mRNA (Table 1) and protein (FIG. 15c) expressions were found to be lower in metastatic tumors lacking AR. Transactivation using p27 5′ promoter-Luciferase assays, however, show that androgens/AR have little influence on p27 transcription. p27 protein stability was assayed and it was observed that p27 was degraded faster in PC3 cells as compared to PC3-AR9 cells in the presence of 1 nM DHT, indicating that androgen/AR up-regulates p27 protein by enhancing its stability.

(c) Androgen/AR Suppresses Metastasis-Related Genes, Such as MMP-9 Via Indirect Modulation of NF-κB Signals.

MMP-9 is known to be involved in tumor metastasis (Wang, X. et al. (2003) Nat. Med. 9, 1313-1317). Higher expression of MMP-9 has been found in bone metastases originating from several primary tumors (Corey, E. et al. (2003) Clin. Cancer Res. 9, 295-306). Both MMP-9 mRNA (Table 1) and protein (FIG. 15d) expressions were found to be higher in metastatic tumors lacking AR. Enzyme assays of MMP-9 in PC3-AR9 cells further show that 1 nM DHT can suppress and 1 μM antiandrogen HF can restore the gelatinase activity of MMP-9 (FIG. 15d). Transactivation assays using MMP-9 5′ promoter-Luciferase assays, however, show that androgen/AR has little influence on MMP-9 transcription. Early reports suggested that MMP-9 is regulated by NF-κB (Eberhardt, W., et al. (2000) J. Immunol. 165, 5788-5797) and androgen/AR can down-regulate NF-κB in prostate cancer cells (Altuwaijri, S. et al. (2003) Cancer Res. 63, 7106-7112). FIG. 13d shows the increased MMP-9 activity can be enhanced via addition of 10 nM TPA, a NF-κB inducer, yet further addition of 1 μg Parth, a NF-κB inhibitor, can then reduce the MMP-9 activity (FIG. 15d). Furthermore, 1 nM DHT can significantly reduce NF-κB expression and addition of 1 μM HF can reverse the DHT suppression effect in PC3-AR9 cells. Therefore androgen/AR can suppress MMP-9 activity via interruption of NF-κB signals.

(d) Androgen/AR Suppresses Akt Activity Via Modulating Akt's Upstream Signals, that are Related to Metastasis/Invasion Genes, Such as Epidermal Growth Factor Receptor (EGFR), Insulin-Like Growth Factor b Chain (IGF-b), Interleukin-6 (IL-6), and Tumor Necrosis Factor-α (TNF-α).

Early reports suggested the activation of Akt signals cause prostate tumor invasion (Enomoto, A. et al. (2005) Dev. Cell 9, 389-402). Interestingly, several reports documented that the four metastasis/invasion-related genes described in Table 1 (EGFR, IGF-b, IL-6, and TNF-α) are able to activate Akt activity (Mizokami, A., et al. (1994) Mol. Endocrinol. 8, 77-88; Yeh, S. et al. (2003) J. Exp. Med. 198, 1899-1908; Corey, E. et al. (2002) Prostate 52, 20-33; Pilatus, U. et al. (2000) Neoplasia 2, 273-279). Previous reports also demonstrated that suppression of androgen/AR activity results in the activation of Akt (Yuan, S. et al (1993) Cancer Res. 53, 1304-1311; Webb, M. M et al. (999) Carcinogenesis 20, 1185-1192; Heitzer, M. D. & DeFranco, D. B. (2006) Cancer Res. 66, 7326-7333; Yeh, S. et al. (2003) J. Exp. Med. 198, 1899-1908; Nagabhushan, M., et al. (1996) Cancer Res. 56, 3042-3046; Goater, J. J., et al. (2002) J. Orthop. Res. 20, 169-173). Thus, loss of the AR in metastatic tumors results in the activation of Akt, possibly via enhancing Akt upstream signals, such as EGFR, IGF-b, IL-6, and TNF-a. As expected, Western blot assay with anti-phospho-Akt (Ser473), show Akt activity is higher in both PLN-pes-ARKO and PC-3 metastatic tumors lacking AR (FIG. 15e).

(9) Molecular Mechanisms by which Stromal AR Promotes Prostate Metastatic Tumor Invasion

Compared to dissecting the mechanisms of why epithelial AR functions in an opposite manner by suppressing prostate metastasis as described above, it is relatively easy in here to explain why stromal AR promotes prostate metastasis. Knockdown of AR via AR-siRNA in WPMY1-ARsi cells result in the decreased mRNA expression of TGFβ1, TGFβ2, TGFβ3, VEGF, and SDF1 (FIG. 15f), which were studied well for their vital roles in the promotion of prostate metastasis (Derynck, R., et al. (2001) Nat. Genet. 29, 117-129; Jennbacken, K., et al. (2005) Prostate 65, 110-116; Taichman, R. S., et al. (2002) Cancer Res 62, 1832-1837) via stromal-epithelial interaction. Early studies also documented well that stromal TGFβs might play key roles in the promotion of prostate metastasis via enhancing angiogenesis and inhibiting immune cells. It is therefore reasonable to believe that stromal AR promotes prostate metastasis via modulation of those metastasis key factors from stromal cells.

b) Conclusion

Herein through four separate means from mice and human prostate cancer cell models (metastatic tumors from pes-ARKO-TRAMP mice, primary culture cells from mice PLN tumors, human CWR22R-AR^{+/-} cells and human PC3-AR9 cells) it is disclosed that loss of epithelial AR signaling enhances invasiveness and metastatic potential. These findings seem at odds with the classic concept with current clinical treatment to prostate cancer that believe androgens/AR stimulates prostate cancer proliferation and progression ((1967) Surg. Gynecol. Obstet. 124:1011-1017; Messing, E. M., et al. (1999) N. Engl. J. Med. 341, 1781-1788). One possible explanation for this odd can that even in metastatic sites, ARs in neighboring stromal cells continue to stimulate malignant prostate epithelial cells until ADT, by silencing the ARs in stroma, places the disease into remission, which is eventually reversed when continued ADT silences the prostate epithelial AR's metastasis Suppressor function, leading to “androgen independent” disease progression. The final outcome of the AR's influence on prostate tumor progression depends upon the balance of these two contrasting roles (stromal AR as stimulator versus epithelial AR as suppressor for metastasis) and the data indicates that stromal AR plays more dominant roles than epithelial AR at early stage of prostate cancer. The implication of these conclusions challenges the current therapeutic approaches, because they imply that ADT as currently utilized, ultimately induces resistance to its own therapeutic effects.

c) Methods

(1) Cell Culture, Plasmids, and Reagents

Human prostate cancer cell lines CWR22R-AR+/+, CWR22R-AR+/−, PC3-v, PC3-AR2 and PC3-AR9 were maintained in RPMI 1640 media with 10% fetal calf serum, 25 U/ml penicillin and 25 μg/ml streptomycin. DHT and HF were from Sigma. Antibodies to AR(C-19), NEP, Cox-2, p27, MMP-9, and actin (Santa Cruz Biotechnology) and Total Akt and p-Akt (473) (Cell Signaling) were used. The natural promoter-driven AR plasmid was constructed by inserting a 3.6 kb hAR promoter with hAR 5′-UTR, and full-length AR cDNA into the pIRES plasmid, and placed the expression of AR under control of the 3.6 kb proximal AR promoter region cloned into pIRES. Neomycin resistant cells were selected by incubation with 500 μg G418/ml.

(2) Construction of Mouse pBabe-AR, hARpcDNA3 and Human AR-siRNA.

Oligonucleotides (CGGAATTCGTGGAAGCT (SEQ ID NO: 11) and GAAAGATCTACATCAGTAG-AGG (SEQ ID NO: 12)) were used to clone an AR fragment from mouse prostate cDNA library using PCR. The retroviral vector pBabe-AR was constructed by ligating the BamHI/Bgl II digested PCR product into BamHI/Bgl II-digested pBabe-GFP vector, PCR-generated human AR cDNA fragments into pcDNA3 vector (Invitrogen), full-length AR cDNA, and 310-bp 3′-UTRs followed by 280-bp bovine GH poly(A) signals into pBlueScript sk(−) vector (Stratagene). The human AR small interfering RNA (siRNA) expression vector that expresses an siRNA-targeting AR in mammalian cells was constructed by digesting and inserting double-strained polynucleotide 5′-GTCGGGCCCTATCCCAGTCCCACTTGCTCGAGCAAGTGGGACTGGGATAGGGCT TTTTGAATT-CGC-3′ (SEQ ID NO: 13) into the ApaI-EcoRI site of a DNA-based vector BS/U6 (Wu, X., et al. (2001) Mech. Dev. 101, 61-69).

(3) Invasion Assays

The invasion assays used Boyden chambers (Becton Dickinson) with 6.4-μm inserts and 8 μm pores. Matrigel Matrix (100 g/cm² surface area) was placed in medium (1:5 dilution) on the inner layer of the invasion chamber and incubated at 37° C. for 30 min before adding the cells. The chambers were placed with or without Matrigel into the wells of 24-well culture plates and added cells in 500 μl aliquots to the inner side of chambers, incubated chambers for 20 hr at 37° C. in a 5% CO₂ atmosphere in the presence and absence of 1 nM DHT, and harvested for cell invasion and migration assays, which were quantified by standard MTT assays as described (Attiga, F. A., et al. (2000) Cancer Res. 60, 4629-4637).

(4) In Vitro Bone-Wafer Resorption and Osteoclastogenesis Assay.

PC-3 and PC-3(AR)9 cells were added to neonatal rat calvarial bone cells (osteoclasts and stromal cells) that were cultured on bone wafers, treated for ten days in the presence of 10 nM parathyroid hormone (PTH) (Bachem), and replaced the media every two days. After 10 days, the wafers were scraped, dried, stained with toluidine blue, and examined them at 40× magnification under a light microscope. A digital camera was used to capture an image of the wafer, traced pits on the surface of the wafer, and the enclosed area determined using Osteometrics software. After 10 days, the wafers were scraped, dried, and stained them for TRAP, using the Leukocyte acid phosphatase kit (Sigma). To quantified osteoclast formation the number of multinucleated TRAP-positive cells was counted as described (Goater, J. J., et al. (2002) J. Orthop. Res. 20, 169-173).

(5) Generation of Transgenic Mice

To generate pes-ARKO mice, ARRPB2-Cre transgenic mice (Wu, X., et al. (2001) Mech. Dev. 101, 61-69) (C57BL/6N, from NIH) were mated with mice (C57BL/6J) containing the conditional AR allele (floxed AR)(Yeh, S. et al. (2002) Proc. Natl. Acad. Sci. USA 99, 13498-13503). To generate pes-ARKO/T857A AR mice, the three transgenic mice, ARRPB2-Cre mice (C57BL/6N), floxed AR mice (C57BL/6J), and T857A AR mice (FVB) (gift from Dr. N. Greenburg, FHCRC, Seattle, Wash.) were interbred. TRAMP (C57BL/6-TRAMP×FVB) and Probasin Cre (Pb-Cre) (C57BL/6) mice were obtained from Jackson Laboratory.

(6) Statistics

Data was presented as the mean L standard deviation (SD). Comparisons between groups were made using a two-sided Student's t test. P values *P<0.05, **P<0.01, ***P<0.001 were considered significant. Survival curves were analyzed by Kaplan-Meier analysis and log-rank tests.

### 3. Example 3

### Loss of Epithelial Androgen Receptor Promotes Prostate Cancer Progression

Tumorigenesis in TRAMP mice is driven by the expression of the SV40 early gene T antigen (T-ag) under control of a minimal probasin promoter. Although there are some concerns about the relevance of this model to human prostate cancer because of the suppression of p53 and unusually high neuroendocrine cell population in TRAMP tumors, this model remains an excellent animal model to study prostate tumor progression because these tumors can be initiated from normal tissue, and tumor progression resembles that of human prostate cancer in that it develops from prostatic intraepithelial neoplasia (PIN) to low grade and then to high grade cancers. Importantly, when TRAMP mice are crossed with AR knockout (ARKO) mice the probasin continues to be expressed (from the 1^{st} day to the 5^{th} week after birth), while the AR starts to be knocked out at the 5^{th} week and this continues until the 24^{th} week with little AR expressed by that time. Thus, there is enough of a time window for T-ag, which is driven by the probasin promoter, to be expressed (for the first 5 weeks of life). This observation also is consistent with early reports showing that TRAMP mice still develop prostate tumors even when the mice were castrated at 4 weeks of age. Using probasin-Cre (Pb-Cre) to knockout the prostate epithelial AR in TRAMP mice (with suppressed AR expression starting a week 5 and increasing through week 24), therefore provides an excellent animal model to study how loss of the prostate epithelial AR influences prostate cancer progression.

a) Result:

(1) Epithelial AR Functions as Suppressor and Stromal AR Functions as Stimulator for the Prostate Cancer Progression in Nude Mice Xenographed with Co-Cultured Epithelial and Stromal Cells.

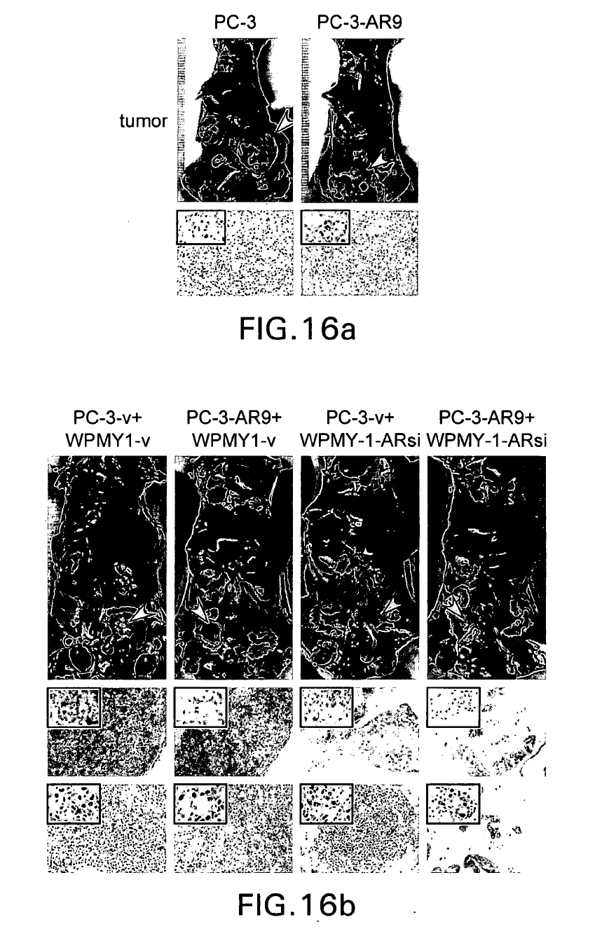

To study AR roles in prostate cancer progression, functional human AR cDNA driven by human AR natural promoter was stably transfected into PC3 cells (named as PC3-AR9). AR transactivation assay demonstrated androgen can stimulate AR activity in these PC3-AR9 cells. PC3-AR9 cells and the parent PC3 cells that stably transfected vector only (named as PC3-v) were injected orthotopically into anterior prostate of nude mice. Interestingly, significantly bigger primary prostate tumors were found in 12 weeks old mice injected with PC3-v cells as compared to those with PC3-AR9 cells (FIG. 16a), indicating addition of functional AR in PC3-AR9 cells results in the suppression of prostate cancer progression.

This unexpected result is against classic concept in the prostate field that believes prostate AR should function as stimulator and not suppressor for prostate cancer progression. These findings were confirmed via another approach with co-culture of epithelial and stromal cells: PC3-v or PC3-AR9 were co-cultured with stromal WPMY1-v cells that expressed functional AR and injected orthotopically these co-cultured cells into anterior prostate of nude mice. The results are consistent with FIG. 16a showing addition of AR in PC3-AR9 cells result in the smaller prostate tumor in 12 weeks mice (FIG. 16b).

AR-siRNA that can effectively knockdown endogenous AR was stably transfected into WPMY1 cells (named WPMY1-ARsi) and co-cultured with either PC3-v or PC3-AR9 cells and injected orthotopically into anterior prostate of nude mice. The results show knockdown AR in stromal WPMY1-ARsi cells result in the suppression of primary prostate tumor growth (PC3-v+WPMY1-v vs PC3-v+WPMY1-ARsi) and knock-in AR in PC3-AR9 cells result in the promotion of primary prostate tumor growth (PC3-v+WPMY1-v vs PC3-AR9+WPMY1). HE staining with tumor malignance also showed primary prostate tumor are more poor differentiation and more malignant in PC3-AR9+WPMY1-Arsi cells. Cell growth assay with proliferation marker Ki67 further confirms the above phenotype observation showing stromal AR functions as stimulator to promote prostate cancer progression in nude mice

Together, both result from FIGS. 16a and 16b clearly demonstrated that epithelial AR functions as suppressor and stromal AR functions as stimulator for the prostate cancer progression.

(2) Generation and Confirmation of Pes-ARKO-TRAMP Mice that Lack AR Only in Prostate Epithelium

As all above data from in vitro cell co-culture system together with in vivo mice models, were all generated from human prostate cancer cells, use of mice that spontaneously developed prostate tumor as another in vivo animal model to prove the above conclusion was of interest. First, pes-ARKO mice that lack AR only in prostate epithelium were generated and demonstrated that loss of epithelial AR in these pes-ARKO mice result in increased prostate cell proliferation. Also, tgn-ARKO mice lacking AR only in prostate stromal smooth muscle were generated and demonstrated that loss of stromal smooth muscle AR in these tgn-ARKO mice result in decreased prostate cell proliferation.

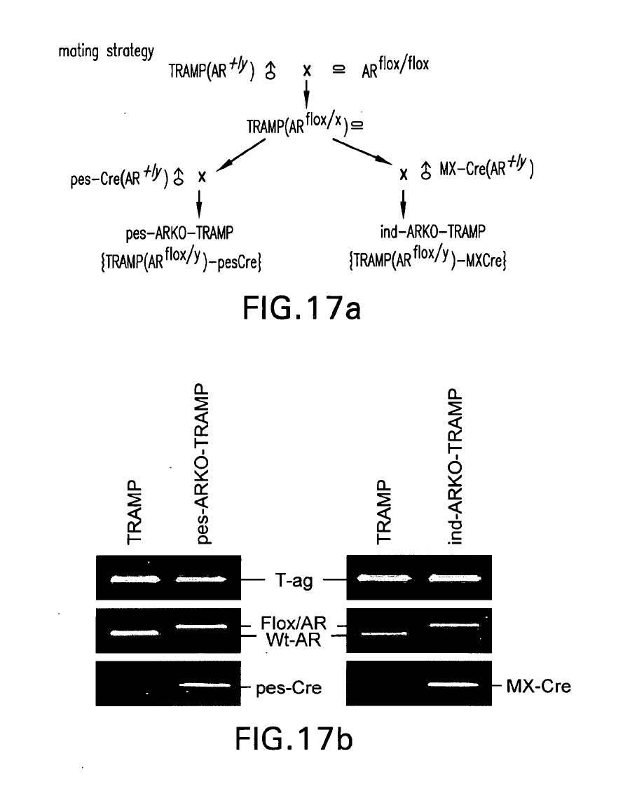

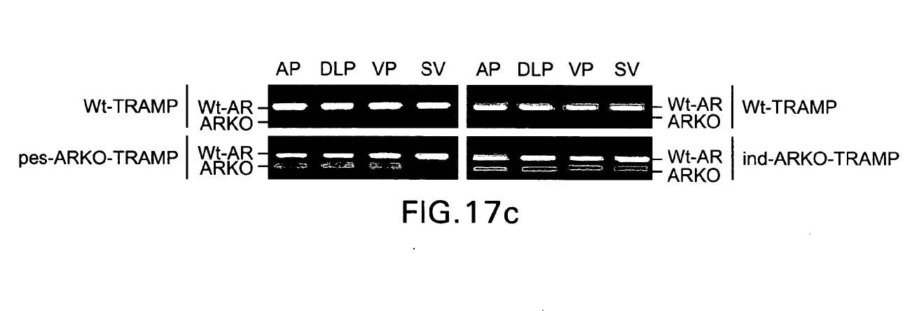

Results from these pes-ARKO mice and tgn-ARKO mice indicated epithelial AR is suppressor and stromal AR is stimulator for normal prostate proliferation. Those floxed/AR mice were used to generate pes-ARKO-TRAMP mice that can spontaneously developed prostate tumor. Female flox/AR(C57BL/6/128) mice were mated with TRAMP (C57BIJ6/TRAM×FVB) mice to generate flox/AR-TRAMP(C57BL/6/129×TRAMP-FVB) mice, and then crossed with Pb-Cre (C57BL/6) mice to generate pes-ARKO-TRAMP (C57BL/6/129×TRAMP-FVB) mice that lack the AR only in prostate epithelium (FIG. 17a).

The pes-ARKO-TRAMP mice were genotyped by PCR from tail snip DNA as described previously¹⁰. As shown in FIG. 17b, both wild type (Wt)-TRAMP and pes-ARKO-TRAMP mice expressed T-ag (FIG. 17b), upper panel), whereas only pes-ARKO-TRAMP mice expressed floxed AR (FIG. 17b, middle panel) and Pb-cre (FIG. 17b, lower panel) bands. mRNA levels were analyzed via PCR from anterior prostate (AP), dorsolateral prostate (DLP), ventral prostate (VP), and seminal vesicles (SV) with primers specific for deleted exon 2 of the AR and demonstrated that AR-exon 2 are excised in AP, DLP and VP from pes-ARKO-TRAMP mice, but not in Wt-TRAMP mice (FIG. 17c).

(3) Generation and Confirmation of Ind-ARKO-TRAMP Mice that Knockdown AR in Prostate.

An inducible knockout system was applied to generate ind-ARKO-TRAMP mice that can knockdown prostate AR (both in epithelium and stroma) via mating female flox/AR-TRAMP(C57B/6/129×TRAMP-FVB) mice with Mx-Cre (C57BL/6/FVB) mice (FIG. 17a). Injection of interferon X into ind-ARKO-TRAMP mice then induces the knockdown of AR in various tissues, including prostate.

To verify the genotype, PCR had been carried out using tail snip DNA as templates. As shown in FIG. 17b, both wild type (Wt)-TRAMP and ind-ARKO-TRAMP mice expressed T-ag (FIG. 17b, upper panels), whereas only ind-ARKO-TRAMP mice expressed floxed AR (FIG. 17b, middle panel) and Mx-Cre (FIG. 17b, lower panel) bands. The knockdown of AR in ind-ARKO-TRAMP mice was further confirmed at mRNA level by RT-PCR via detecting the mRNA deletion of AR exon2 in different organs, such as anterior prostate (AP), dorsolateral prostate (DLP), ventral prostate (VP), seminal vesicle (SV), liver, spleen, and testis (FIG. 17c).

(4) AR Expression in pes-ARKO-TRAMP and ind-ARKO-TRAMP Mice

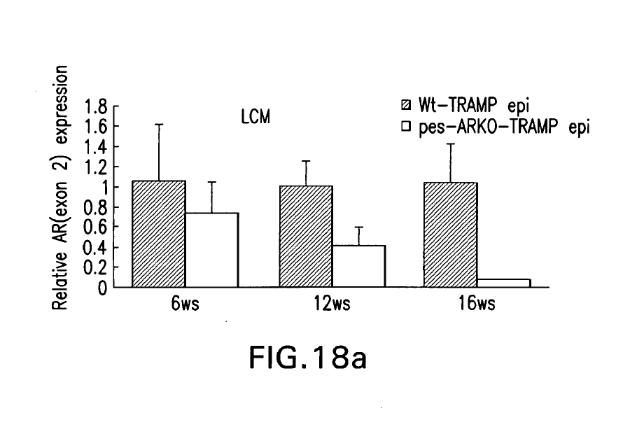

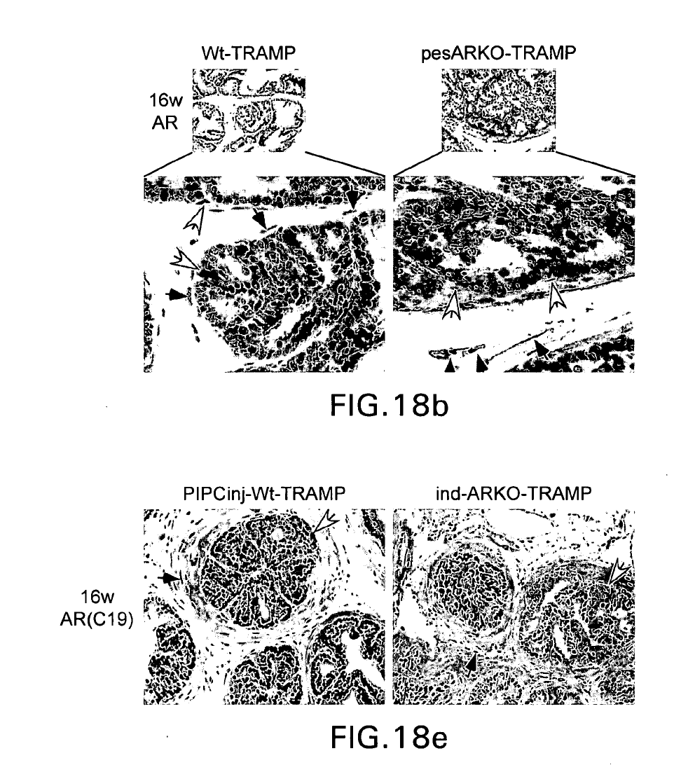

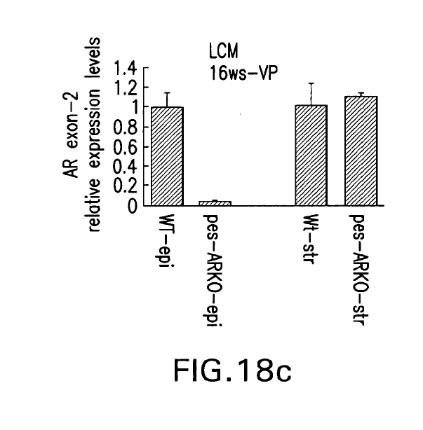

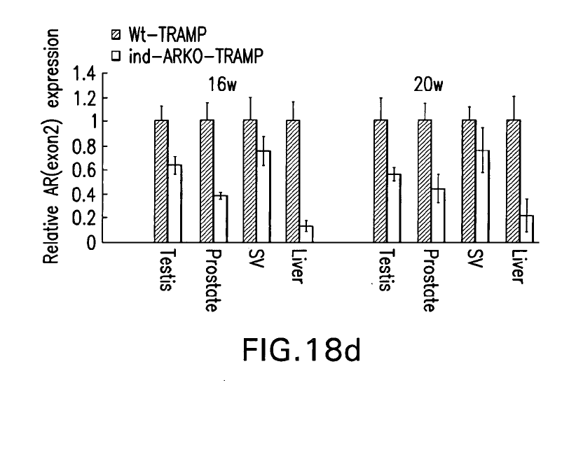

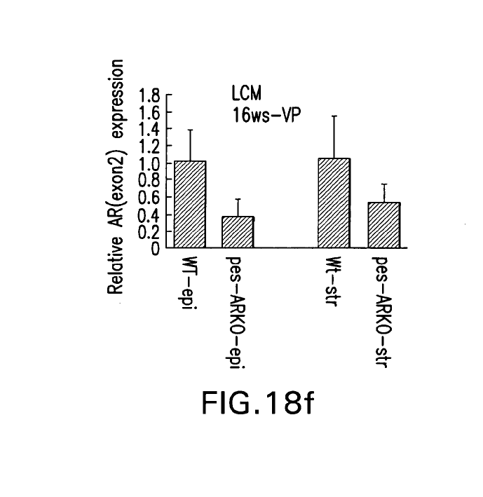

To monitor the AR knockout efficiency in pes-ARKO-TRAMP mice, quantitative realtime RT-PCR was utilized to measure the expression of AR exon2 mRNA that extracted from prostate epithelium via laser capture microdissection (LCM). Results from FIG. 18a showed AR mRNA was knocked out 25%, 50% and over 90% in 6 wks, 12 wks, and 16 wks of pes-ARKO-TRAMP mice. Immunohistochemical staining of AR expression in prostate from 16 wks pes-ARKO-TRAMP mice confirmed the loss of AR in the prostate epithelium, including luminal and basal cells, but not in stromal cells (FIG. 18b). Quantitative realtime RT-PCR from ventral prostate isolated via LCM also confirmed the loss of AR mRNA in epithelium but not in stromal cells (FIG. 18c).

For the monitor of knockdown efficiency in ind-ARKO-TRAMP mice that injected PIPC at 12 wks for the period of either 4 wks or 8 wks, it was found that AR mRNA was knockdowned at 40-50% in prostate, 40% in testis, 20% in seminal vesicle, and 80% in liver (FIG. 18d). Immunohistochemical staining of AR further confirmed the knockdown of AR in 16 wks of ind-ARKO-TRAMP mice that injected PIPC for the period of 4 weeks (FIG. 18e). Quantitative realtime RT-PCR of AR mRNA from LCM-isolated epithelium or stroma in 16 wks of ind-ARKO-TRAMP mice also confirmed the loss of 60% AR mRNA in epithelium and 50% AR mRNA in stromal cells (FIG. 18f).

(5) Prostate Size and Serum Testosterone Changes in pes-ARKO-TRAMP and ind-ARKO-TRAMP Mice.

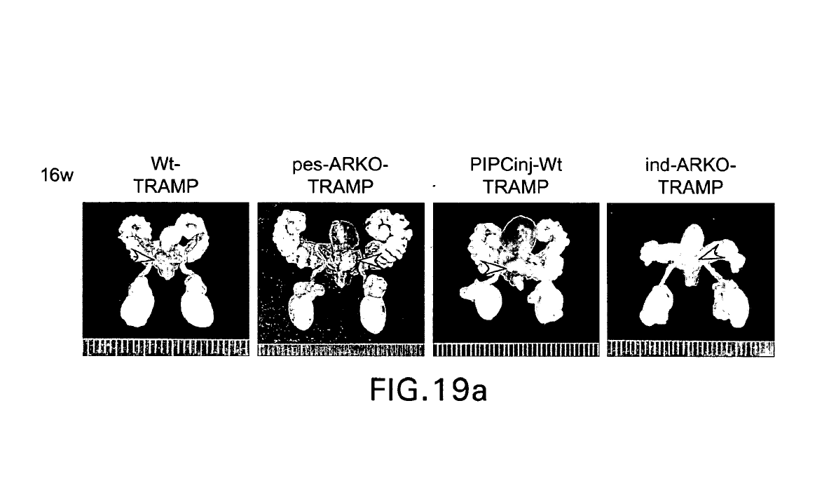

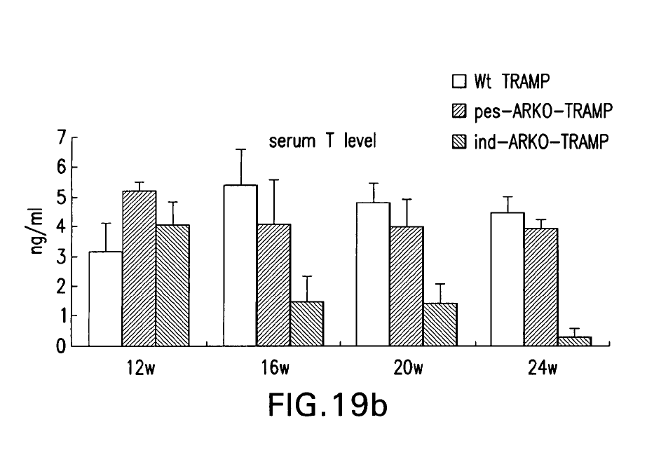

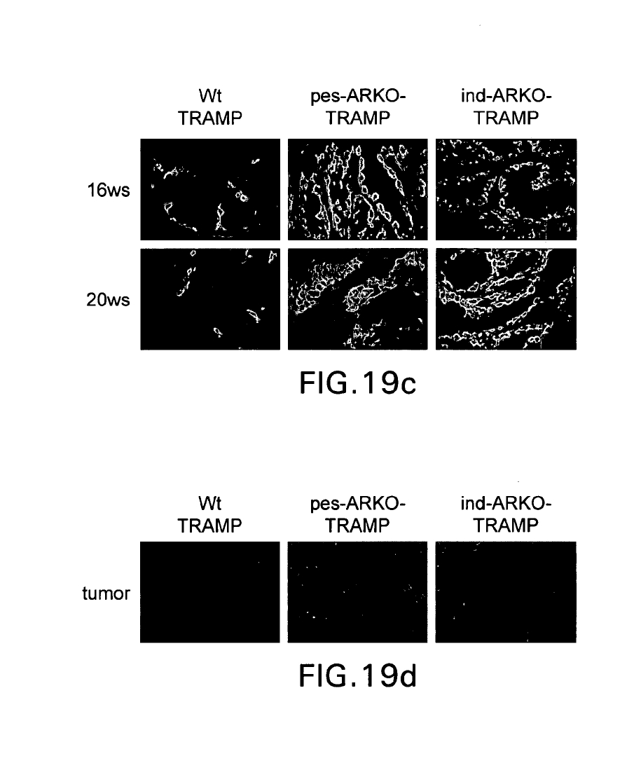

The overall of reproductive organs, except prostate in pes-ARKO-TRAMP mice is larger, are similar between pes-ARKO-TRAMP mice and their wild type (WT) littermate (FIG. 19a). In contrast, ind-ARKO-TRAMP mice had smaller reproductive organs, including prostate as compared to their WT littermate (FIG. 19a). Serum testosterone remains compatible between pes-ARKO-TRAMP mice and their WT littermates. However, serum testosterone was reduced from 16 to 24 weeks of ind-ARKO-TRAMP mice that injected PIPC at 12 weeks. Together, results from FIGS. 19a and 19b indicated that knockout AR in prostate epithelium result in the larger prostate with little change of serum testosterone in pes-ARKO-TRAMP mice and inducible knockdown of AR in ind-ARKO-TRAMP mice result in the smaller prostate with lower serum testosterone.

(6) Larger Population of Intermediate Cells Found in the Prostate Epithelium of pes-ARKO-TRAMP and ind-ARKO-TRAMP Mice.

Among the three major cells (reserve stem cells, intermediate cells and secretory luminal cells) within the prostate epithelium, knockdown of epithelial AR results in the loss of most of secretory luminal cells. In contrast, CK5/CK8 double positive intermediate cells were increased in both pes-ARKO-TRA and ind-ARKO-TRAMP mice (FIG. 19d). This conclusion is further supported by immunofluorescent straining of another intermediate cell marker CD44 showing higher CD44 expression in pes-ARKO-TRAMP and ind-ARKO-TRAMP mice (FIG. 19e). Together, results from FIG. 19c to 19e indicated knockout AR in epithelium result in the cell population changes with more intermediate cells and much less secretory luminal cells in prostate of pes-ARKO-TRAMP and ind-ARKO-TRAMP mice.

(7) Increased Vs Decreased Prostate Cancer Progression in pes-ARKO-TRAMP and ind-ARKO TRAMP Mice

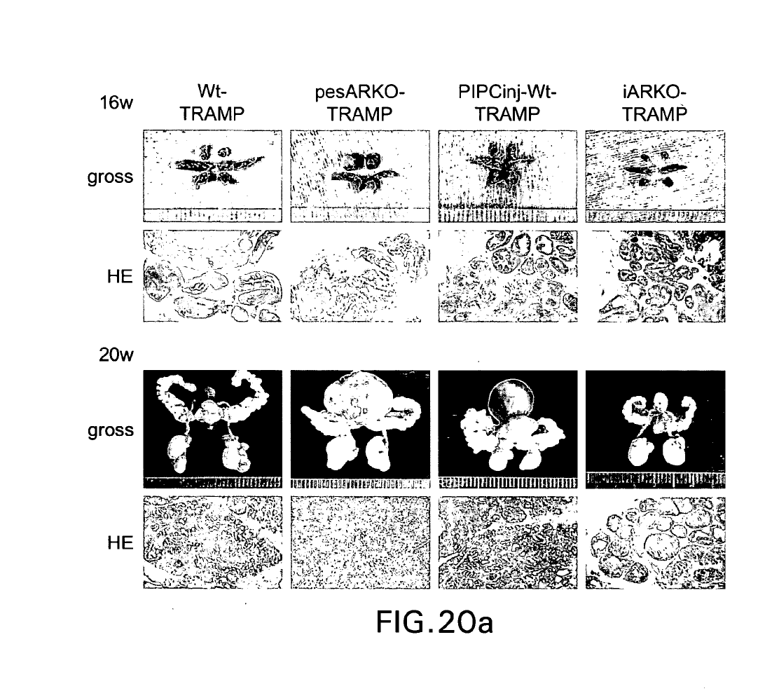

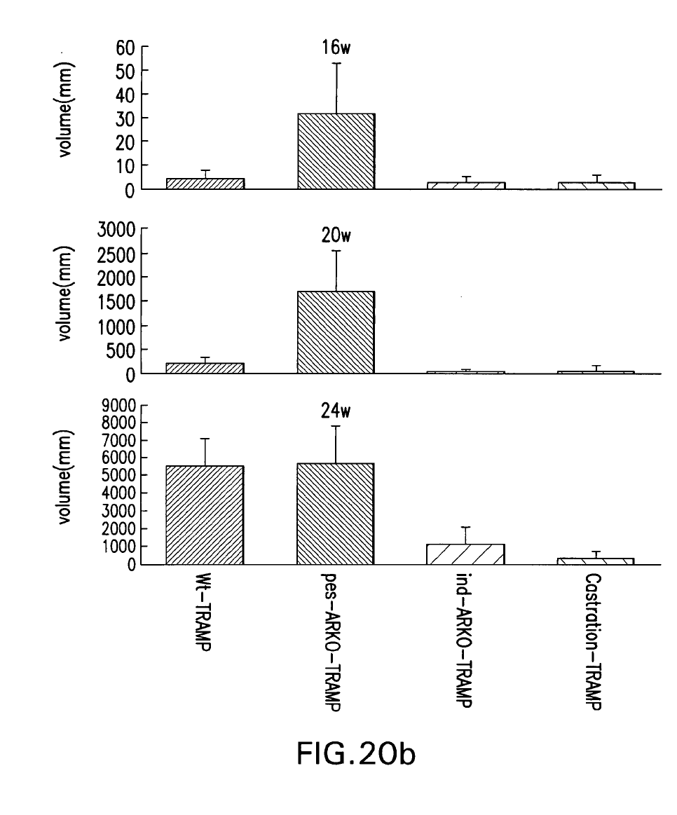

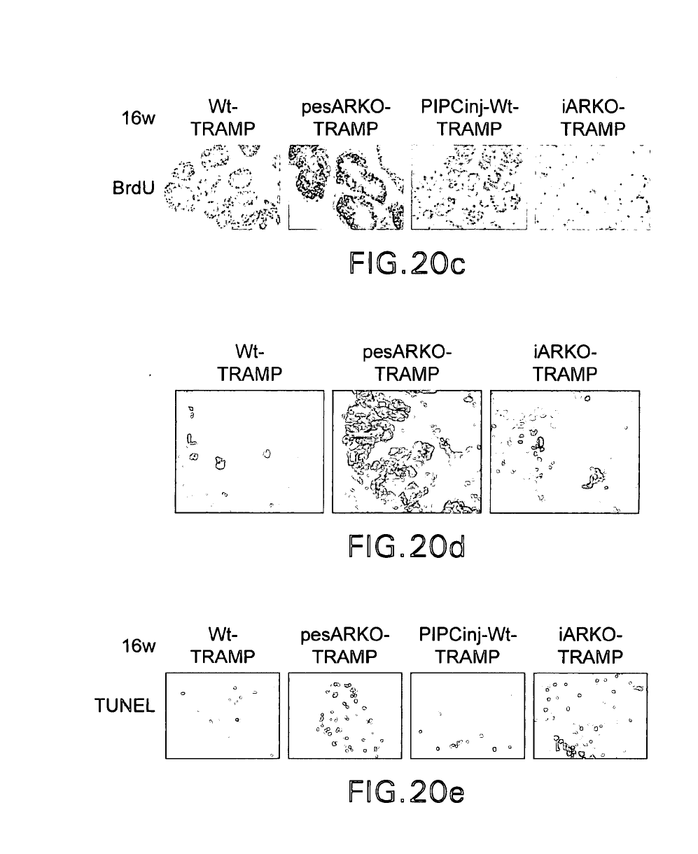

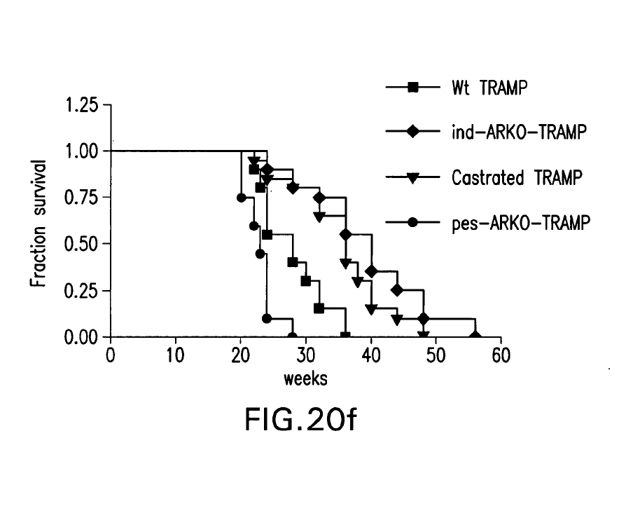

Examining whether altered cell populations with increased intermediate cells within epithelium via knockout of epithelial AR influence prostate cancer progression, larger primary prostate tumors were found in 16, 20 and 24 weeks old pes-ARKO-TRAMP mice than the WT littermate. In contrast, smaller primary prostate tumors were observed in 24 weeks of ind-ARKO-TRAMP mice that received injections EPIC at 12 weeks in comparison to their WT littermate. (FIG. 20a-b). HE staining also found that primary prostate tumors in pes-ARKO-TRAMP mice were poorly differentiated as compared to their WT littermates at the age of 16 weeks or 20 weeks (FIG. 20a, HE), indicating tumor in pes-ARKO-TRAMP mice bear more aggressive behavior as compared to their wt littermate.

Notably, the smaller primary prostate tumor found in ind-ARKO-TRAMP mice were also poorly differentiated as compared to their WT littermate (FIG. 20a).

To correlate the increased size with growth rate in the primary prostate tumors, both proliferation rate via BrdU staining and apoptosis rate via TUNEL assay were assayed. Substantially higher BrdU staining was observed in primary prostate tumors of pes-ARKO-TRAMP mice as compared to those in WT littermates (FIG. 20c). In contrast, less BrdU staining was found in primary prostate tumors of ind-ARKO-TRAMP mice as compared to those in their WT littermate (FIG. 20c). Double staining of another proliferation marker Ki67 with CK-5-positive intermediate cells also confirm BrdU data showing higher proliferation activity in primary prostate tumor of pes-ARKO-TRAMP mice and less proliferation in ind-ARKO-TRAMP mice as compared to those in their WT littermates (FIG. 20c). Using TUNEL assay, a higher apoptosis rate was seen in primary prostate tumor of pes-ARKO-TRAMP mice and a lower apoptosis rate was seen in primary prostate tumor of ind-ARKO-TRAMP mice as compared to those in their WT littermates (FIG. 20d). Together, this indicates a larger and more aggressive primary prostate tumor with higher proliferation and apoptosis rate in pes-ARKO-TRAMP mice and smaller and less aggressive primary prostate tumor with lower proliferation and apoptosis rate in ind-ARKO-TRAMP mice as compared to those in their WT littermate. The larger and more aggressive primary prostate tumors in pes-ARKO-TRAMP mice result in earlier death as compare to their WT littermate (FIG. 20e) In contrast, the smaller and less aggressive primary prostate tumor in ind-ARKO-TRAMP mice results in longer survival as compared to their WT littermate (FIG. 20e).

Together, results from both human prostate cancer cells with either knock-in or knockdown AR in the co-culture system (FIG. 16) and mice with either knockout epithelial AR or knockdown with epithelial and stromal AR (FIG. 20) all demonstrate that epithelial AR functions as suppressor and stromal AR functions as stimulator for prostate cancer progression. Furthermore, stromal AR plays a more dominant role than epithelial AR so that simultaneously knockdown epithelial and stromal AR results in the suppression of prostate cancer progression.

(8) Molecular Mechanisms why Loss of Epithelial AR in pes-ARKO-TRAMP Mice Promotes Cell Proliferation

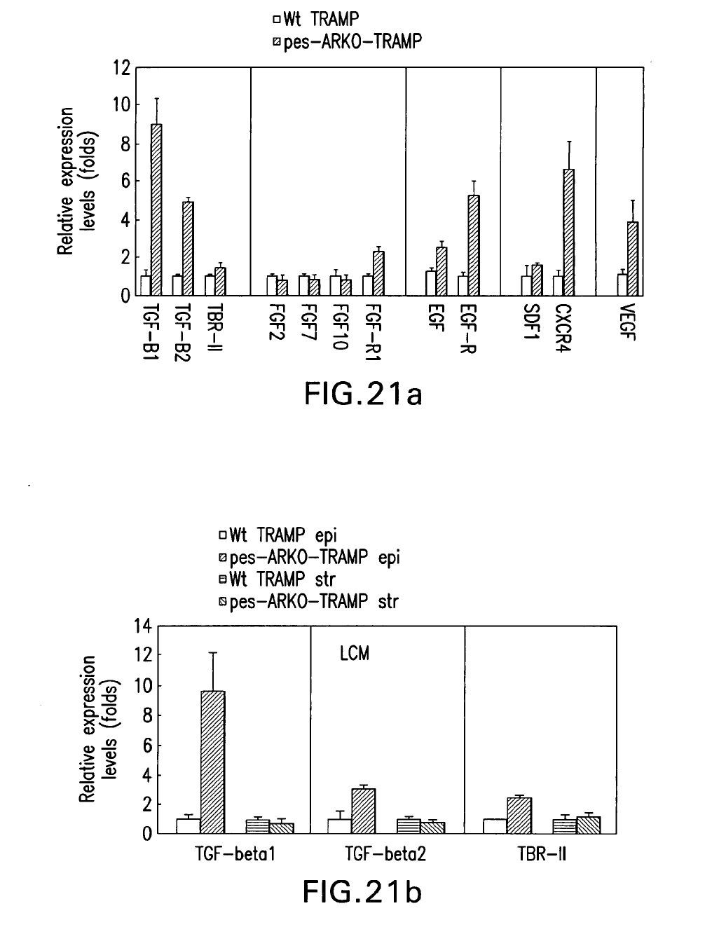

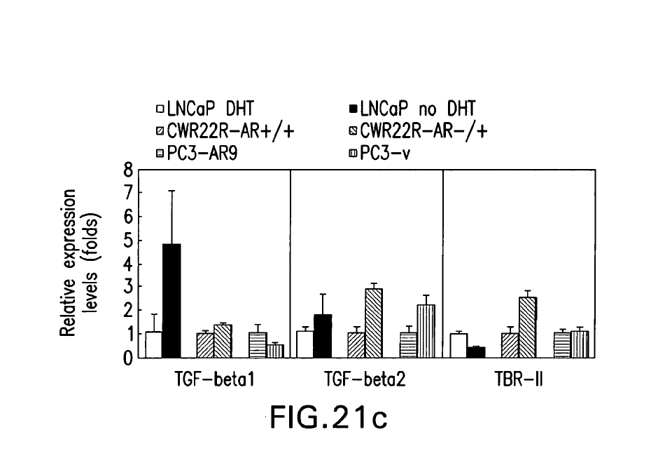

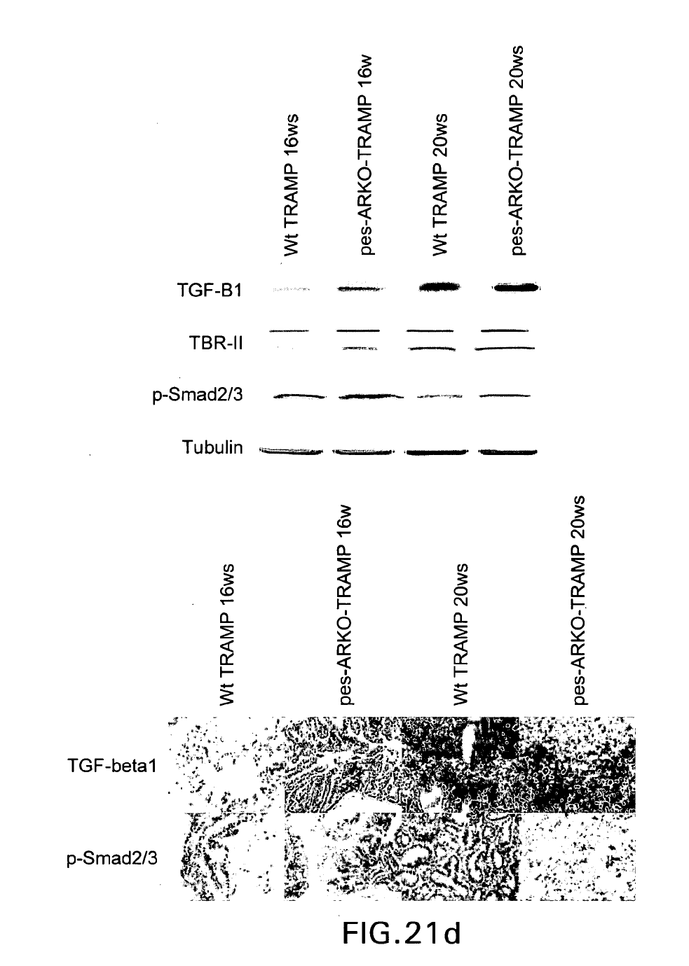

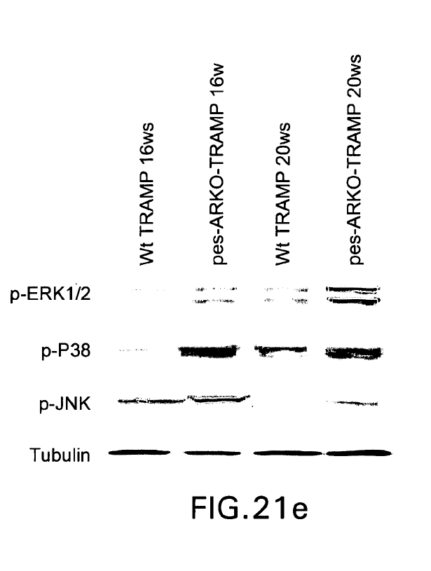

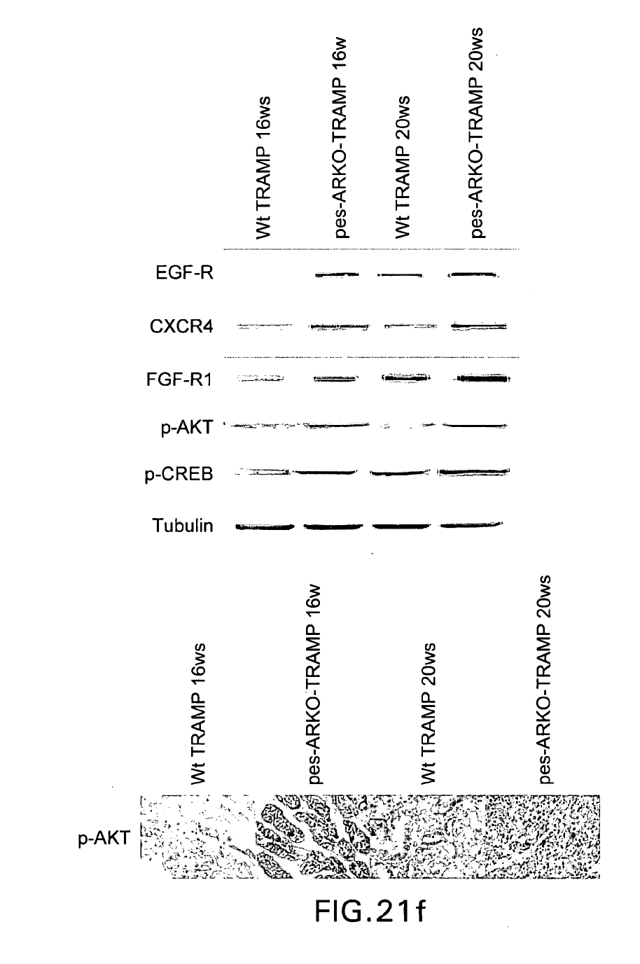

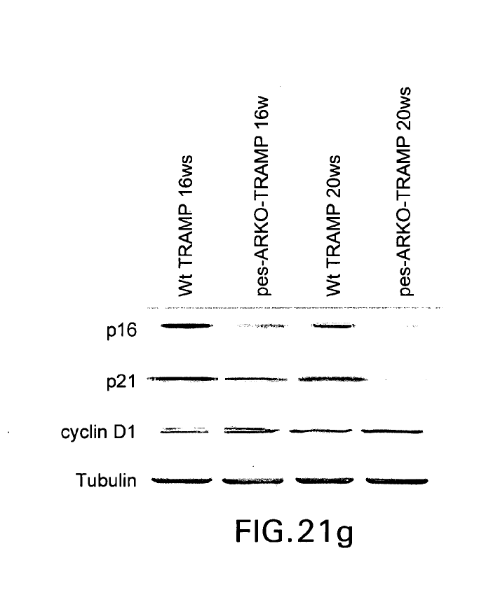

To dissect the molecular mechanisms why loss of epithelial AR results in the promotion of prostate cancer progression, several signal pathways that linked to promotion of cell proliferation were screened. The results from real-time PCR quantitation indicates that signals for TGFβs, EGF, SDF1, VEGF and FGF receptor only were increased in ventral prostate (that already developed PIN) from 16 weeks of pes-ARKO-TRAMP as compared to those from WT littermates (FIG. 21a) Next, TGFβ signals were studied that had been previously studied for their negative influence on the epithelial growth. The results from real-time PCR quantitation confirm the increased mRNA expression for TGFβ1, TGFβ2 and their receptor TβR-II from LCM-isolated prostate epithelium of 16 weeks of pes-ARKO-TRAMP mice (FIG. 21b). In contrast, mRNA expression of TGFβ1, TGFβ2 and TβR-II from LCM-isolated prostate stroma is compatible between pes-ARKO-TRAMP and their WT littermates (FIG. 21b). These results were further confirmed from 2 human prostate cancer cells: data from FIG. 21c showed knockdown of AR in CWR22R cells or deprived of DHT in LNCaP cells all result in the increased TGFβ1 and TGFβ2 mRNA expression. Western blot and immunostaining data also found the increased TGFβ1 protein expression in ventral prostate from 16 and 20 weeks of pes-ARKO-TRAMP mice as compared to those in WT littermates (FIG. 21d).

Increased TGFβ signals in LCM-isolated epithelial of pes-ARKO-TRAMP mice results in the increased its downstream target phospho-Smad2/3 expression in the cytoplasma. (FIG. 21e). To further examine if accumulation of increased cytosol phospho-Smad2/3 expression in prostate epithelium of pes-ARKO-TRAMP mice goes through MAPK signals to promote cell proliferation, increased phospho-Smad2/3 expression was found which was accompanied by increased phospho-Erk1/2, phospho-JNK and phospho-38 in prostate isolated from 16 and 20 weeks of pes-ARKO-TRAMP mice as compared to those in their wt littermate (FIG. 21e).

For the other increased signals shown in FIG. 21a, immunostaining was used to further confirm the increased protein expression for the EGF-R, EGF-R1 and CXCR4 (FIG. 21f) in ventral prostate from 16 and 20 weeks of pes-ARKO-TRAMP mice as compared to those in WT littermates. The increased signals from these growth factor pathways results in the higher expression of phospho-AKT and phospho-CREB (FIG. 21f).

Collectively, the increased signals from both TGFβŝphospho-smad2/3̂phospho-Erk1/2/phospho-JNK/phospho38 and EGFR/FGFR/SDF1-CXCR4̂phospho-AKT/phospho-CREB all contribute to the decreased expression of p1⁶ and p21 and increased cyclin D1 expression (FIG. 21g) that result in the increased prostate tumor proliferation in pes-ARKO-TRAMP mice.

(9) Molecular Mechanisms why Stromal AR Functions as Stimulator to Promote Cell Proliferation.

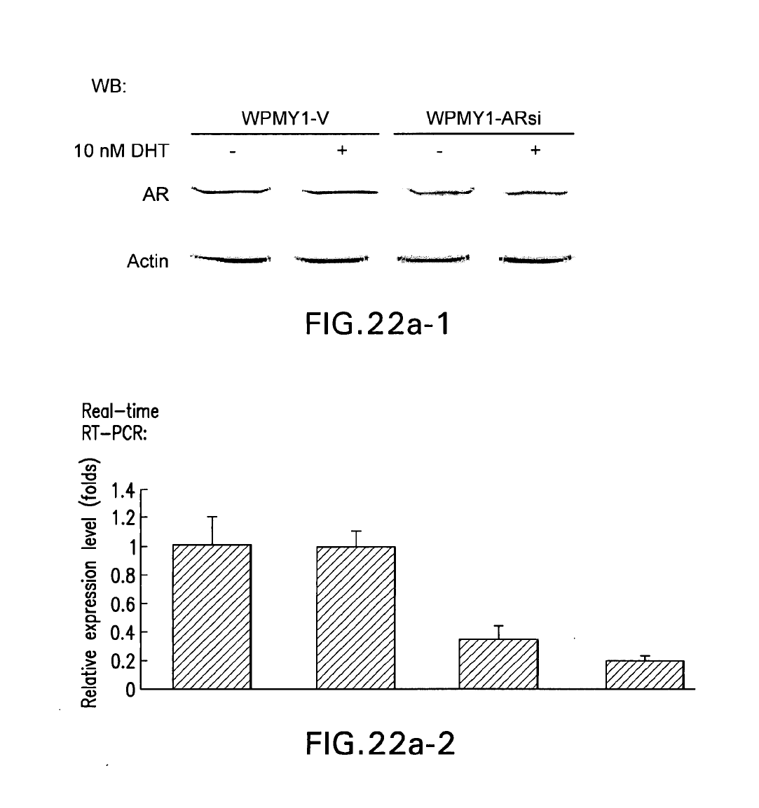

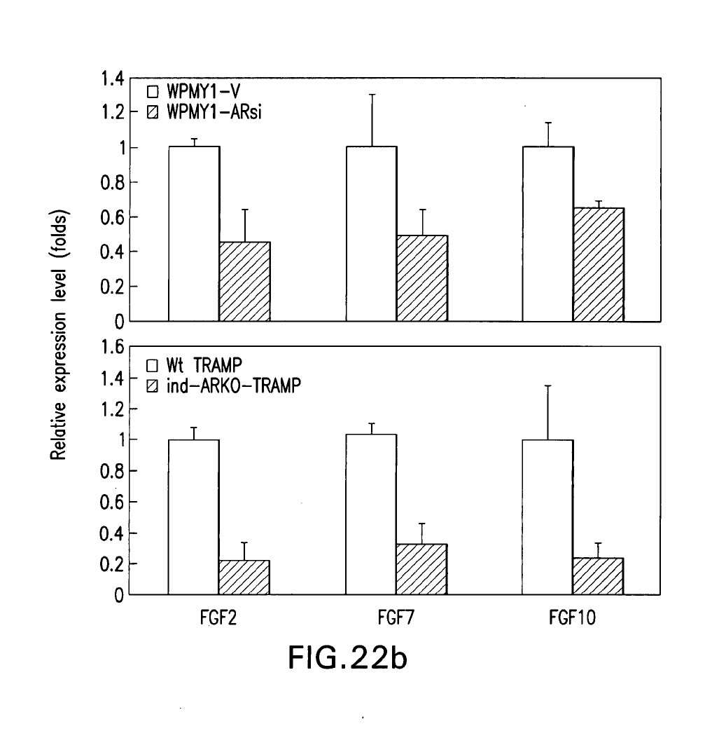

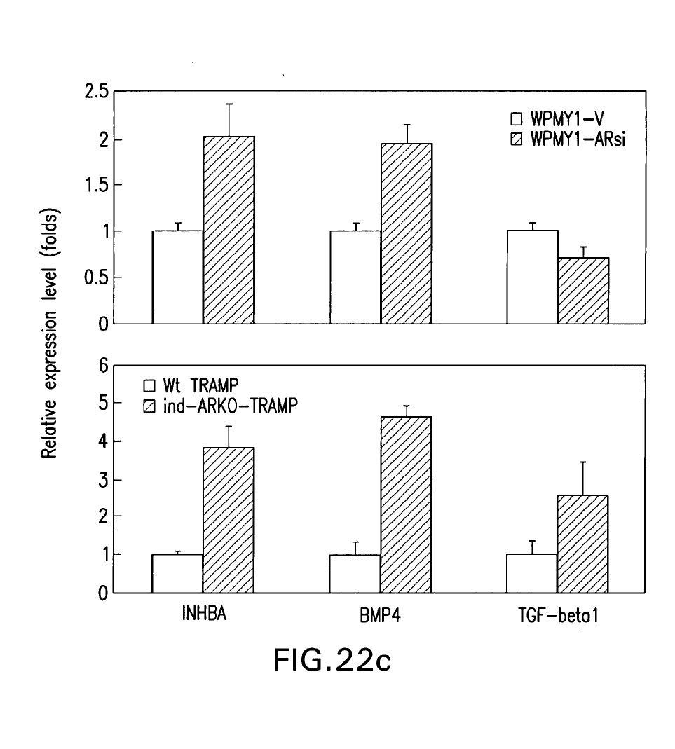

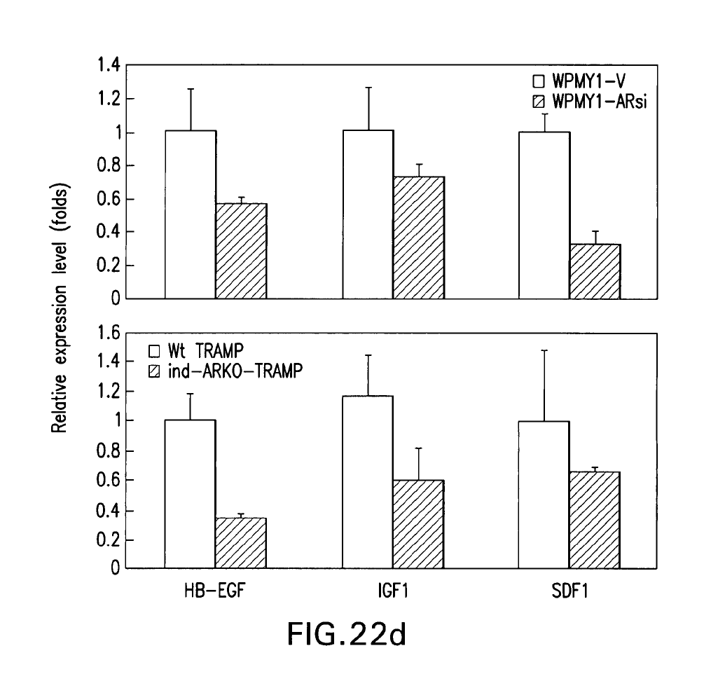

To dissect the mechanisms why stromal AR plays opposite roles to the epithelial AR (stimulator vs suppressor), signals for FGFs, TGFβ1, EGF, and SDF-1 were studied and found to be decreased, instead of increased as found in pes-ARKO-TRAMP mice, in prostate from ind-ARKO-TRAMP mice (FIG. 22b-d). The results from real-time PCR quantitation also confirmed the decreased mRNA expression of FGF2, FGF7, FGF10 (FIG. 22b), as well as HB-EGF and SDF-1 (FIG. 22d) in co-culture of PC3-WPMY1-ARsi as compared to co-culture of PC3-WPMY2-v. Furthermore, decreased expression of IGF1 and increased expression of INHBA and BMP4 in both co-cultured PC3-WPMY-ARsi and ind-ARKO-TRAMP mice (FIG. 22c-d) also support the stimulator roles of stromal AR to promote the prostate tumor progression.

b) Methods

(1) Cell Culture, Plasmids, and Reagents

A CWR22R-AR^{+/-} cell line was generated from parental CWR22R-AR^{+/+} cell line as described previously, using homologous gene recombination strategy to knockdown AR expression. w PC3 cells ere stably transfected with AR cDNA driven by human promoter previously and named this cell line as PC3-AR9. Neomycin resistant cells were selected by incubation with 500 μg G418/ml. Human prostate cancer cell lines CWR22R-AR^{+/+}, CWR22R-AR^{+/-}, PC-3, and PC-3(AR)9 were maintained in RPMI 1640 media with 10% fetal calf serum, 25 U/ml penicillin and 25 μg/ml streptomycin. DHT and HF were from Sigma.

(2) Establishment of Stable Transfected Cell Lines WPMY1-ARsi.

A human AR small interfering RNA (siRNA) expression vector was constructed that expresses an siRNA-targeting AR mRNA sequence 5′-GTCGGGCCCTATCCCAGTCCCACTTGCTCGAGCAAGTGGG-3 (SEQ ID NO: 20) and 5-GCGAATTCAAAAAGCCCTATCCCAGTCCCACTTGCTCGAG-3 (SEQ ID NO: 21) in mammalian cells into the pSuperior vector.

WPMY1 cells, obtained from the American Type Culture Collection, were cultured to the mid- or late-logarithmic phase of growth. After trypsinization, the cells were resuspended and washed twice in 2.5% FBS medium without antibiotics. 400 μl of the cell suspension (10⁷ cells) was transferred into the electroporation cuvettes (VWR), set the voltages of the electroporator (Bio-RAD GENE PULSER II) to 300V and hinge capacity to 950 μF, added 20 μg of total pSuperior-Vector or pSuperior-ARsi DNA to each cuvette, and incubated for 5 min at RT. After pulse charge, the cells were incubated on ice for 5 min and transferred to a 35-mm culture dish. After culturing in complete medium at 37° C. with 5% CO₂ for 72 h, the transfected cells were trypsinized and replated in 5 μg/ml of puromycin to select infected cells. The selection medium was changed every 24 days for 2-3 weeks until colonies of resistant cells formed. By this method, WPMY1-vector (WPMY)-v) and WPMY1-ARsi stable cell lines were established.

(3) Tumorigenesis in the Nude Mice

To investigate whether AR plays a role in prostate tumor growth in vivo, a protocol that introduces PC3 and PC3-AR9 cells directly into anterior prostate of athymic nude mice was utilized After anesthesia, the abdomens of 8 ws old nude mice were surgically open in sterile environments. 5×10⁶ PC3 or PC3-AR9 cells suspended in 100 μl of Matrigel were directly injected into anterior prostate by fine needles. After operation, the abdomens were closed by silk suture stitch. 12 ws following the injection, the mice were sacrificed to harvest the xenograft tumors Tissues were fixed in paraffin.

(4) Generation of Transgenic Mice

To generate pes-ARKO-TRAMP or ind-ARKO-TRAMP mice, TRAMP (C57BL/6-TRAMP×FVB, from Jackson Laboratory) transgenic mice were mated with floxed AR mice (C57BL/6J) containing the conditional AR allele (floxed AR)3, to generate TRAMP-floxAR female mice. Then TRAMP-floxAR female mice were interbred with either ARRPB2-Cre mice¹³ (C57BL/6N, from NIH) or Mx Cre (C57BL/6-FVB, from Jackson Laboratory) to generate the pes-ARKO-TRAMP or ind-ARKO-TRAMP respectively.

(5) Laser Capture Microdissection

Prostate tissues were embedded O.C.T. on dry ice and stored at −80° C. until cutting. 5 μm sections were cut onto plain uncoated glass slides and immediately stained them by HistoGene LCM Frozen Section Staining Kit (from Arcturus) following the manufacturer's instructions. On Pixcell II LCM system, the prostatic epithelium and stroma were laser transferred on to different caps. After microdissection, caps were merged in RNA extraction buffer of PicoPure RNA Isolation Kit (from Arcturus) and RNA was isolated following the manufacturer's instructions. One round of RNA amplification had been done using RiboAmp RNA Amplification Kit (from Arcturus).

(6) RNA Extraction, RT-PCR and Real-Time RT-PCR

Tissues were harvested in Trizol (Invitrogen) and extracted total RNA following the manufacturer's instructions. 5 μg total RNA was reverse transcribed into 20 μl cDNA immediately by the SuperScript m kit (Invitrogen) with oligo-dT primer. PCR and real-time PCR were performed on the MyCycler thermal cycler (Bio-RAD) with 1 μl cDNA amplified by Taq polymerase (Promega) and on the iCycler IQ multicolor real-time PCR detection system with 1/5 μl cDNA amplified by SYBR Green PCR Master Mix respectively.

Primers were designed using Beacon Designer 2 software as follows: AR exon2 (forward: 5′-GGACAGTACCAGGGACCATG-3′, reverse: 5′-TCCGTAGTGACAGCCAGAAG-3′); TGFβ1 (forward: 5′-TAATGGTGGACCGCAACAAC-3′, reverse: 5′-GTATTCCGTCTCCTTGGTTCAG-3′); TGFβ2 (forward: 5′-CAGAGCGGAGGGTGAATG-3′; reverse: 5′-GGCGAAGGCAGCAATTATC-3′); TGFβ3 (forward: 5′-AGGAGTGGACAATGAAGATG-3′, reverse: 5′-TGAGCAGAAGTTGGCATAG-3′); TβR-II; Smad2 (forward: 5′-CTACACCCACTCCATTCC-3′, reverse: 5′-GCAGGTTCCGAGTAAGTAA-3′); Smad3 (forward: 5′-GGGCTTTGAGGCTGTCTA-3′, reverse: 5′-AAGGGTCCATTCAGGTGTA-3′); Smad4 (forward: 5′-CACTATGAGCGGGTTGTC-3′, reverse: 5′-GGTGCTGGTGGCGTTAGA-3′), BMP4; BMP7, INHBA, mCTGF (forward: 5′-ATCTCCACCCGAGTTACCA-3′, reverse: 5′-AACTTAGCCCTGTATGTCTTCA-3′), hCTGF (forward: 5′-AATGCTGCGAGGAGTGGG-3′, reverse: 5′-GGCTCTAATCATAGTTGGGTCT-3′), HB-EGF; mHGF (forward: 5′-AGAGGTACGCTACGAAGTC-3′, reverse: 5′-GCTTGCCATCAGGATTGC-3′), hHGF (forward: 5′-AGGGGCACTGTCAATACCATT-3′, reverse: 5′-CGTGAGGATACTGAGAATCCCAA-3′), MIGF (forward: 5′-GGTGGATGCTCTTCAGTTC-3′, reverse: 5′-TTTGTAGGCTTCAGTGGG-3′); hIGF (forward: 5′-TATTTCAACAAGCCCACAG-3′, reverse: 5′-ATACATCTCCAGCCTCCTTA-3′); NGF, VEGF (forward: 5′-gga aca ccg aca aac cca-3′, reverse: 5′-tcc cca aag cac agc aat-3′); FGF2(forward: 5′-AACGGCGGCTTCTTCCTG-3′, reverse: 5′-TGGCACACACTCCCTTGATAG-3′); FGF7(forward: 5′-TCCTGCCAACTCTGCTCTAC-3′, reverse: 5′-CTTTCACTTTGCCTCGTTTGTC-3′); FGF10 (forward: 5′-CTGCTGTTGCTGCTTCTTG-3′, reverse: 5′-TGACCTTGCCGTTCTTCTC-3′); SFRP1; SDF1 (forward: 5′-CTGTGCCCTTCAGATTGTT-3′, reverse: 5′-GGCGGAGTGTCTTTATGC-3′). β-Actin (forward: 5′-TGTGCCCATCTACGAGGGGTATGC-3′, and reverse: 5′-GGTACATGGTGCCGCCAGACA-3′) was used as internal control. The Δ threshold (CT) values were calculated by subtracting the control CT value from the corresponding β-Actin CT from each time point. The absence of nonspecific amplification products was confirmed by agarose-gel electrophoresis.

(7) Immunohistochemistry

Samples were fixed in 5% neutral buffered formalin and embedded in paraffin. The AR, ARA70 and PSA protein expression level were studied by IHC method in all 4 pairs of samples. Rabbit anti-Ki67, rabbit anti-Tag, rabbit anti-AR(C 19) (Santa Cruz Biotechnology), anti-CK5, anti-CK8, anti-CD44, anti-TGFβ1, anti-pSmad2/3, and anti-pAKT antibodies were used. The bound primary antibody was recognized by the biotinylated secondary antibody (Vector), and visualized by VECTASTAIN ABC peroxidase system (Vector) and peroxidase substrate DAB kit (Vector). The positive staining were semi-quantitated by Image J software.

(8) Immunofluorescence Staining

Samples were fixed in 5% neutral buffered formalin and embedded in paraffin. Sections were incubated overnight at 4° C. with primary antibodies, mouse anti-CK5( ) and chicken anti-CK8 antibody (Abcam). Following 60 min rinse (3×20 min, PBS+1% Triton-X 100), slices were incubated with secondary antibodies (Alexa Fluors, donkey anti-chicken 596 and horse anti-mouse 488) for 1 hr at RT. Slices were rinsed for 60 min (3×20 min), and mounted with Vectashield Mounting Medium H1000 (Vector Laboratories, Burlingame, Calif.) and examined on a fluorescein microscope (Leica).

(9) Western Blot Analysis.

Tissue and cell lysates were prepared in RIPA buffer. Then w protein samples ere separated on SDS-10% PAGE gel and transferred them to a polyvinylidene difluoride membrane. After blocking by 5% non-fat milk and 5% FBS in PBST buffer, the membrane was immunoblotted with the primary antibody, followed by incubation with AP-conjugated second antibody (Santa Cruz). Finally, the membrane was developed by the AP color developing reagents (Bio-RAD). Antibodies to AR(C-19), NEP, Cox-2, p27, MMP-9, and actin (Santa Cruz Biotechnology) and Total Akt and p-Akt (473) (Cell Signaling) were used.

(10) BrdU Incorporation Assay

5′-Bromo-2′-deoxyuridine (BrdU) was bought from Sigma and resolved in DDW in 10 mg/ml concentration. 24 hrs before sacrifice, mice were injected intraperitoneally every 6 hrs for 10 μG BrdU per gram body weight. Following harvest, tissues were embedded in paraffin and labeled following the manufacturer's instructions of BrdU Staining Kit from Zymed Laboratories Inc.

(11) TUNEL Assay

Fluorescein—Frag EL™ DNA Fragmentation Detection Kit was bought from CALBIOCHEM co. Paraffin-embedded tissue sections were labeled following the manufacturer's instructions. The labeled nuclei were counted by using a standard fluorescein filter of 465-495 nm.

(12) Statistics

The data was presented as the mean±standard deviation (SD). Comparisons were made between groups using a two-sided Student's t test. P values *P<0.05, **P<0.01, ***P<0.001 were considered significant. Survival curves were analyzed by Kaplan-Meier analysis and log-rank tests.

### G. REFERENCES

Altuwaijri, S. et al. Interruption of nuclear factor kappaB signaling by the androgen receptor facilitates 12-O-tetradecanoylphorbolacetate-induced apoptosis in androgen-sensitive prostate cancer LNCaP cells. Cancer Res 63, 7106-12 (2003).
Attiga, F. A., Fernandez, P. M., Weeraratna, A. T., Manyak, M. J. & Patierno, S. R. Inhibitors of prostaglandin synthesis inhibit human prostate tumor cell invasiveness and reduce the release of matrix metalloproteinases. Cancer Res 60, 4629-37 (2000).
Baldassarre, G. et al. p27(Kip1)-stathmin interaction influences sarcoma cell migration and invasion. Cancer Cell 7, 51-63 (2005).
Belletti, B. et al. p27(kip1) functional regulation in human cancer: a potential target for therapeutic designs. Curr Med Chem 12, 1589-605 (2005).
Bello, D., Webber, M. M., Kleinman, H. K., Wartinger, D. D. & Rhim, J. S. Androgen responsive adult human prostatic epithelial cell lines immortalized by human papillomavirus 18. Carcinogenesis 18, 1215-23 (1997).
Bhatia-Gaur, R. et al. Roles for Nkx3.1 in prostate development and cancer. Genes Dev 13, 966-77 (1999).
Chang, C. S., Kokontis, J. & Liao, S. T. Molecular cloning of human and rat complementary DNA encoding androgen receptors. Science 240, 324-6 (1988).
Chen, C. D., Welsbie, D. S., Tran, C., Baek, S. H., Chen, R., Wessella, R. Rosenfeld, M. G., Sawyers. C. L. Molecular determinants of resistance to antiandrogen therapy. Nat. Med. 10, 33-39 (2004).
Corey, E. et al. Establishment and characterization of osseous prostate cancer models: intra-tibial injection of human prostate cancer cells. Prostate 52, 20-33 (2002).
Corey, E. et al. Zoledronic acid exhibits inhibitory effects on osteoblastic and osteolytic metastases of prostate cancer. Clin Cancer Res 9, 295-306 (2003).
Cunha, G. R. & Lung, B. The possible influence of temporal factors in androgenic responsiveness of urogenital tissue recombinants from wild-type and androgen-insensitive (Tfm) mice. J Exp Zool 205, 181-93 (1978).
Cunha, G. R. et al. Hormonal, cellular, and molecular regulation of normal and neoplastic prostatic development. J Steroid Biochem Mol Biol 92, 221-36 (2004).
Danielpour, D., Kadomatsu, K., Anzano, M. A., Smith, J. M. & Sporn, M. B. Development and characterization of nontumorigenic and tumorigenic epithelial cell lines from rat dorsal-lateral prostate. Cancer Res 54, 3413-21 (1994).
Derynck, R., Akhurst R. J. & Balmain, A. TGFβ signaling in tumor suppression and cancer progression. Nat. Genet. 29, 117-129 (2001).
Donjacour, A. A. & Cunha, G. R. Assessment of prostatic protein secretion in tissue recombinants made of urogenital sinus mesenchyme and urothelium from normal or androgen-insensitive mice. Endocrinology 132, 2342-50 (1993).
Donjacour, A. A. & Cunha, G. R. The effect of androgen deprivation on branching morphogenesis in the mouse prostate. Develop. Biol. 128, 1-14 (1988).
Eberhardt, W., Huwiler, A., Beck, K. F., Walpen, S. & Pfeilschifier, J. Amplification of IL-1 beta-induced matrix metalloproteinase-9 expression by superoxide in rat glomerular mesangial cells is mediated by increased activities of NF-kappa B and activating protein-1 and involves activation of the mitogen-activated protein kinase pathways 3 Immunol 165, 5788-97 (2000).
Eisenberger, M. A., Blumenstein, B. A., Crawford, E. D., Miller, G., McLeod, D. G., Loehrer, P. J., Wilding, G., Sears, K., Thompson, I. M., Bueschen, A. J., Lowe, B. A. Bilateral orchidectomy with or without flutamide for metastatic prostate cancer. New Engl. J. Med. 339, 1036-1042 (1998).
Enomoto, A. et al. Akt/PKB regulates actin organization and cell motility via Girdin/APE. Dev Cell 9, 389-402 (2005).
Evans, G. S. & Chandler, J. A. Cell proliferation studies in the rat prostate: II. The effects of castration and androgen-induced regeneration upon basal and secretory cell proliferation. Prostate 11, 339-51 (1987).
Festuccia, C. et al. Epithelial and prostatic marker expression in short-term primary cultures of human prostate tissue samples. Int J Oncol 26, 1353-62 (2005).
Fluchter, S. H., Weiser, R., Gamper, C. The role of hormonal treatment in prostate cancer. Recent Results Cancer Res. 175, 211-237 (2007).
Goater, J. J., O'Keefe, R. J., Rosier, R. N., Puzas, J. E. & Schwarz, E. M. Efficacy of ex vivo OPG gene therapy in preventing wear debris induced osteolysis. J Orthop Res 20, 169-73 (2002).
Greenberg, N. M. et al. Prostate cancer in a transgenic mouse. Proc Natl Acad Sci USA 92, 3439-43 (1995).
Greenberg, N. M. et al. The rat probasin gene promoter directs hormonally and developmentally regulated expression of a heterologous gene specifically to the prostate in transgenic mice. Mol Endocrinol 8, 230-9 (1994).
Han, G. et al. Hormone status selects for spontaneous somatic androgen receptor variants that demonstrate specific ligand and cofactor dependent activities in autochthonous prostate cancer. J Biol Chem 276, 11204-13 (2001).
Han, G. et al. Mutation of the androgen receptor causes oncogenic transformation of the prostate. Proc Natl Acad Sci USA 102, 1151-6 (2005).
Hayward, S. W. et al. Epithelial development in the rat ventral prostate, anterior prostate and seminal vesicle. Acta Anat (Basel) 155, 81-93 (1996).
He, W. W. et al. A novel human prostate-specific, androgen-regulated homeobox gene (NKX3.1) that maps to 8p21, a region frequently deleted in prostate cancer. Genomics 43, 69-77 (1997).

Heitzer, M. D. & DeFranco, D. B. Hic-5/ARA55, a LIM Domain-containing Nuclear Receptor Coactivator Expressed in Prostate Stromal Cells. Cancer Res. 66, 7326-7333 (2006).
Hill, R. E., de Avila, D. M., Bertrand K. P., Greenberg N. M. & Reeves J. J. Immunization Against Luteinizing Hormone-Releasing Hormone Fusion Proteins Does Not Decrease Prostate Cancer in the Transgenic Adenocarcinoma Mouse Prostate Model. Exp. Biol. Med. 228, 818-822 (2003).
Ito, H. et al. Remodeling of cortical bone allografts mediated by adherent rAAV-RANKL and VEGF gene therapy. Nat Med 11, 291-7 (2005).
Jennbacken, K., Vallbo, C., Wang, W., Damber, J. E. Expression of vascular endothelial growth factor C (VEGF-C) and VEGF receptor-3 in human prostate cancer is associated with regional lymph node metastasis. Prostate 65, 110-116 (2005).
Kim, M. J. et al. Nkx3.1 mutant mice recapitulate early stages of prostate carcinogenesis. Cancer Res 62, 2999-3004 (2002).
Kuhn, R. Schwenk, F., Aguet, M., Rajewsky, K. Inducible gene targeting in mice. Science 269, 1427-1429 (1995).
Kwong, J., Xuan, J. W., Chan, P. S., Ho, S. M. & Chan, F. L. A comparative study of hormonal regulation of three secretory proteins (prostatic secretory protein-PSP94, probasin, and seminal vesicle secretion II) in rat lateral prostate. Endocrinology 141, 4543-51 (2000).
Li, R. et al. High level of androgen receptor is associated with aggressive clinicopathologic features and decreased biochemical recurrence-free survival in prostate: cancer patients treated with radical prostatectomy. Am J Surg Pathol 28, 928-34 (2004).
Lin, H. K. et al. Suppression versus induction of androgen receptor functions by the phosphatidylinositol 3-kinase/Akt pathway in prostate cancer LNCaP cells with different passage numbers. J Biol Chem 278, 50902-7 (2003).
Messing, E. M., Manola, J., Sarosdy, M., Wilding, G., Crawford, E. D., Trump, D. Immediate hormonal therapy compared with observation after radical prostatectomy and pelvic lymphadenectomy in men with node-positive prostate cancer. N. Engl. J. Med. 341, 1781-1788 (1999).
Mirosevich, J. et al. Androgen receptor expression of proliferating basal and luminal cells in adult murine ventral prostate. J Endocrinol 162, 341-50 (1999).
Mizokami, A., Yeh, S. Y. & Chang, C. Identification of 3′,5′-cyclic adenosine monophosphate response element and other cis-acting elements in the human androgen receptor gene promoter. Mol Endocrinol 8, 77-88 (1994).
Murillo, H., Huang, H., Schmidt, L. J., Smith, D. I. & Tindall, D. J. Role of PI3K signaling in survival and progression of LNCaP prostate cancer cells to the androgen refractory state. Endocrinology 142, 4795-805 (2001).
Nagabhushan, M., Miller, C. M., Pretlow, T. P. et al. CWR22: the first human prostate cancer xenograft with strongly androgen-dependent and relapsed strains both in vivo and in soft agar. Cancer Res. 56, 3042-3046 (1996).
Nelson, J. B. & Carducci, M. A. Small bioactive peptides and cell surface peptidases in androgen-independent prostate cancer. Cancer Invest 18, 87-96 (2000).
Papandreou, C. N. et al. Neutral endopeptidase 24-11 loss in metastatic human prostate cancer contributes to androgen-independent progression. Nat Med 4, 50-7 (1998).
Pilatus, U. et al. Imaging prostate cancer invasion with multi-nuclear magnetic resonance methods: the Metabolic Boyden Chamber. Neoplasia 2, 273-9 (2000).
Quigley, C. A. et al. Androgen receptor defects: historical, clinical, and molecular perspectives. Endocr Rev 16, 271-321 (1995).
Reddig, P. J. & Juliano, R. L. Clinging to life: cell to matrix adhesion and cell survival. Cancer Metastasis Rev 24, 425-39 (2005).
Saunders, M. A., Sansores-Garcia, L., Gilroy, D. W. & Wu, K. K. Selective suppression of CCAAT/enhancer-binding protein beta binding and cyclooxygenase-2 promoter activity by sodium salicylate in quiescent human fibroblasts. J Biol Chem 276, 18897-904 (2001).
Sehgal, I. et al. Neurotensin is an autocrine trophic factor stimulated by androgen withdrawal in human prostate cancer. Proc Natl Acad Sci USA 91, 4673-7 (1994).
Shannon, J. M. & Cunha, G. R. Autoradiographic localization of androgen binding in the developing mouse prostate. Prostate 4, 367-373 (1983).
Signoretti, S. & Loda, M. Defining cell lineages in the prostate epithelium. Cell Cycle 5, 138-41 (2006).
Signoretti, S. et al. p63 is a prostate basal cell marker and is required for prostate development. Am J Pathol 157, 1769-75 (2000).
Sugimura, Y., Cunha, G. R. & Donjacour, A. A. Morphogenesis of ductal networks in the mouse prostate. Biol. Reprod. 34, 961-971 (1986).
Suzuki, H., Ueda, T., Ichikawa, T. & Ito, H. Androgen receptor involvement in the progression of prostate cancer. Endocr Relat Cancer 10, 209-16 (2003).
Taichman, R. S., Cooper, C., Keller, E. T., Pienta, K. J., Taichman, N. S., McCauley, L. K. Use of the stromal cell-derived factor-1/CXCR4 pathway in prostate cancer metastasis to bone. Cancer Res 62, 1832-1837 (2002).
Takeda, H., Mizuno, T. & Lasnitzki, I. Autoradiographic studies of androgen-binding sites in the rat urogenital sinus and postnatal prostate. J. Endocrinol. 104, 87-92 (1985).
Tanimura, Y. et al. Tumor necrosis factor alpha promotes invasiveness of cholangiocarcinoma cells via its receptor, TNFR2. Cancer Lett 219, 205-13 (2005).
The Veterans Administration Co-operative Urological Research Group. Treatment and survival of patients with cancer of the prostate. Surg. Gynecol. Obstet. 124:1011-1017 (1967).
Wang, X. et al. Lipoprotein receptor-mediated induction of matrix metalloproteinase by tissue plasminogen activator. Nat Med 9, 1313-7 (2003).
Wang, Y. et al. A human prostatic epithelial model of hormonal carcinogenesis. Cancer Res 61, 6064-72 (2001).
Wang, Y., Hayward, S., Cao, M., Thayer, K. & Cunha, G. Cell differentiation lineage in the prostate. Differentiation 68, 270-279 (2001).
Webb, M. M. et al. A human prostatic stromal myofibroblast cell line WPMY-1: a model for stromal-epithelial interactions in prostatic neoplasia. Carcinogenesis 20, 1185-1192 (1999).
Wilson, J. D., George, F. W. & Renfree, M. B. The endocrine role in mammalian sexual differentiation. Recent Prog Horm Res 50, 349-64 (1995).
Wu, X. et al. Generation of a prostate epithelial cell-specific Cre transgenic mouse model for tissue-specific gene ablation. Mech Dev 101, 61-9 (2001).
Yang, A. et al. p63 is essential for regenerative proliferation in limb, craniofacial and epithelial development. Nature 398, 714-8 (1999).
Yang, L. et al. Interleukin-6 differentially regulates androgen receptor transactivation via PI3K-Akt, STAT3, and MAPK, three distinct signal pathways in prostate cancer cells. Biochem Biophys Res Commun 305, 462-9 (2003).
Yeh, S. et al. Abnormal mammary gland development and growth retardation in female mice and MCF7 breast cancer cells lacking androgen receptor. J Exp Med 198, 1899-908 (2003).
Yeh, S. et al. Generation and characterization of androgen receptor knockout (ARKO) mice: an in vivo model for the study of androgen functions in selective tissues. Proc Natl Acad Sci USA 99, 13498-503 (2002).

Yi, H. K. et al. Impact of PTEN on the expression of insulin-like growth factors (IGFs) and IGF-binding proteins in human gastric adenocarcinoma cells. Biochem Biophys Res Commun 330, 760-7 (2005).
Yi, H. K. et al. Impact of PTEN on the expression of insulin-like growth factors (IGFs) and IGF-binding proteins in human gastric adenocarcinoma cells. Biochem Biophys Res Commun 330, 760-7 (2005).
Yuan, S. et al. Androgen-induced inhibition of cell proliferation in an androgen-insensitive prostate cancer cell line (PC-3) transfected with a human androgen receptor complementary DNA. Cancer Res 53, 1304-11 (1993).

| H. Sequences | |
|---|---|
| 1. Genbank Accession No. X80172. M. musculus gene for androgen- | |
| receptor 5′ untranslated region. | |
| 1 | ctgcagcttg ttctttaatg tcaggagact ctcccttctg cttgtcctgg tgggccctgg |
| 61 | ggggagcggg gagggaatac ctaagagcaa ttggtagctg gtacttctaa tgcctcttcc |
| 121 | tcctccaacc tccaagagtc tgttttggga ttgggttcag gaatgaaatt ctgcctgtgc |
| 181 | taacctcctg gggagccggt agacttgtct gttaaaaatc gcttctgctt ttggagccta |
| 241 | aagcccggtt ccgaaaaaca agtggtattt aggggaaaga gggglcltca aaggctacag |
| 301 | tgagtcattc cagccttcaa ccatactacg ccagcactac gttctctaaa gccactctgc |
| 361 | gctagcttgc ggtgagggga ggggagaaaa ggaaagggga ggggagggga ggggagggag |
| 421 | aaaggaggtg ggaaggcaga gaggccggct gcgggggcgg gaccgactca caaactgttc |
| 481 | gatttcgttt ccacctccca gcgccccctc ggagatccct aggagccagc ctgctgggag |
| 541 | aaccagaggg tccggagcaa acctggaggc tgagagggca tcagagggga aaagactgag |
| 601 | ctagccactc cagtgccata cagaagctta agggacgcac cacgccagcc ccagcccagc |
| 661 | gacagccaac gcctgttgca gagcggcggc ttcgaagccg ccgcccagga gctgcccttt |
| 721 | cctcttcggt gaagtttcta aaagctgcgg gagactcaga ggaagcaagg aaagtgtccg |
| 781 | gtaggactac ggctgccttt gtcctcttcc cctctaccct taccccctcc tgggtcccct |
| 841 | ctccaggagc tgactaggca ggctttctgg ccaaccctct cccctacacc cccagctctg |
| 901 | ccagccagtt tgcacagagg taaactccct ttggctgaga gtaggggagc ttgttgcaca |
| 961 | ttgcaaggaa ggcttttggg agcccagaga ctgaggagca acagcacgcc caggagagtc |
| 1021 | cctggttcca ggttctcgcc cctgcacctc ctcctgcccg cccctcaccc tgtgtgtggt |
| 1081 | gttagaaatg aaaagatgaa aaggcagcta gggtttcagt agtcgaaagc aaaacaaaag |
| 1141 | ctaaaagaaa acaaaaagaa aatagcccag ttcttatttg cacctgcttc agtggacttt |
| 1201 | gaatttggaa ggcagaggat ttcccctttt ccctcccgtc aaggtttgag catcttttaa |
| 1261 | tctgttcttc aagtatttag agacaaactg tgtaagtagc agggcagatc ctgtcttgcg |
| 1321 | cgtgccttcc tttactggag actttgaggt tatctgggca ctccccccac ccaccccccc |
| 1381 | tcctgcaagt tttcttcccc ggagcttccc gcaggtgggc agctagctgc agatactaca |
| 1441 | tcatcagtca ggagaactct tcagagcaag agacgaggag gcaggataag ggaattc |
| 2. Genbank Accession No. X59591. Mouse gene for androgen receptor | |
| promoter region. | |
| 1 | ctgcagcttg ttctttaatg tcaggagact ctcccttctg cttgtcctgg tgggccctgg |
| 61 | ggggagcggg gagggaatac ctaagagcaa ttggtagctg gtacttctaa tgcctcttcc |
| 121 | tcctccaacc tccaagagtc tgttttggga ttgggttcag gaatgaaatt ctgcctgtgc |
| 181 | taacctcctg gggagccggt agacttgtct gttaaaaatc gcttctgctt ttggagccta |
| 241 | aagcccggtt ccgaaaaaca agtggtattt aggggaaaga ggggtcttca aaggctacag |
| 301 | tgagtcattc cagccttcaa ccatactacg ccagcactac gttctctaaa gccactctgc |
| 361 | gctagcttgc ggtgagggga ggggagaaaa ggaaagggga ggggagggga ggggagggag |
| 421 | aaaggaggtg ggaaggcaga gaggccggct gcgggggcgg gaccgactca caaactgttc |
| 481 | gatttcgttt ccacctccca gcgccccctc ggagatccct aggagccagc ctgctgggag |
| 541 | aaccagaggg tccggagcaa acctggaggc tgagagggca tcagagggga aaagactgag |
| 3. Genbank Accession No. X59590. Mouse gene for androgen receptor, | |
| 3′ UTR. | |
| 1 | cccaagcgct agtgttctgt tctctttttg taatcttgga atcttttgtt gctctaaata |
| 61 | caattaaaaa tggcagaaac ttgtttgttg gaatacatgt gtgactcttg gtttgtctct |
| 121 | gcgtctggct ttagaaatgt catccattgt gtaaaatact ggcttgttgg tctgccagct |
| 181 | aaaacttgcc acagcccctg ttgtgactgc aggctcaagt tattgttaac aaagagcccc |
| 241 | aagaaaagct gctaatgtcc tcttatcacc attgttaatt tgttaaaaca taaaacaatc |
| 301 | taaaatttca gatgaatgtc atcagagtlc ttttcattag ctctttttat tggctgtct |
| 4. Genbank Accession No. X59592. Mouse protein for androgen | |
| receptor. | |
| MEVQLGLGRVYPRPPSKTYRGAFQNLFQSVREAIQNPGPRHPEAANIAPPGACLQQRQETSPRRRRRQQ | |
| HTEDGSPQAHIRGPTGYLALEEEQQPSQQQAASEGHPESSCLPEPGAATAPGKGLPQQPPAPPDQDDSA | |
| APSTLSLLGPTFPGLSSCSADIKDILNEAGTMQLLQQQQQQQQHQQQHQQHQQQQEVISEGSSARAREA | |
| TGAPSSSKDSYLGGNSTISDSAKELCKAVSVSMGLGVEALEHLSPGEQLRGDCMYASLLGGPPAVRPTP | |
| CAPLPECKGLPLDEGPGKSTEETAEYSSFKGGYAKGLEGESLGCSGSSEAGSSGTLEIPSSLSLYKSGA | |
| LDEAAAYQNRDYYNFPLALSGPPHPPPPTHPHARIKLENPLDYGSAWAAAAAQCRYGDLGSLHGGSVAG | |
| PSTGSPPATTSSSWHTLFTAEEGQLYGPGGGGGSSSPSDAGPVAPYGYTRPPQGLTSQESDYSASEVWY | |
| PGGVVNRVPYPSPNCVKSEMGPWMENYSGPYGDMRLDSTRDHVLPIDYYFPPQKTCLICGDEASGCHYG | |
| ALTCGSCKVFFKRAAEGKQKYLCASRNDCTIDKFRRKNCPSCRLRKCYEAGMTLGARKLKKLGNLKLQE | |
| EGENSNAGSPTEDPSQKMTVSHIEGYECQPIFLNVLEAIEPGVVCAGHDNNQPDSFAALLSSLNELGER | |
| QLVHVVKWAKALPGFRNLHVDDQMAVIQYSWMGLMVFAMGWRSFTNVNSRMLYFAPDLVFNEYRMHKSR | |
| MYSQCVRMRHLSQEFGWLQITPQEFLCMKALLLFSIIPVDGLKNQKFFDELRMNYIKELDRIIACKRKN | |
| PTSCSRRFYQLTKLLDSVQPIARELHQFTFDLLIKSHMVSVDFPEMMAEIISVQVPKILSGKVKPIYFH | |
| TQ” | |
| 5. Genbank Accession No. X59592. Mouse mRNA for androgen receptor. | |
| 1 | gcttcccgca ggtgggcagc tagctgcaga tactacatca tcagtcagga gaactcttca |
| 61 | gagcaagaga cgaggaggca ggataaggga attcggtgga agctacagac aagctcaagg |
| 121 | atggaggtgc agttagggct gggaagggtc tacccacggc ccccatccaa gacctatcga |
| 181 | ggagcgttcc agaatctgtt ccagagcgtg cgcgaagcga tccagaaccc gggccccagg |
| 241 | caccctgagg ccgctaacat agcacctccc ggcgcctgtt tacagcagag gcaggagact |
| 301 | agcccccggc ggcggcggcg gcagcagcac actgaggatg gttctcctca agcccacatc |
| 361 | agaggcccca caggctacct ggccctggag gaggaacagc agccttcaca gcagcaggca |
| 421 | gcctccgagg gccaccctga gagcagctgc ctccccgagc ctggggcggc caccgctcct |
| 481 | ggcaaggggc tgccgcagca gccaccagct cctccagatc aggatgactc agctgcccca |
| 541 | tccacgttgt ccctgctggg ccccactttc ccaggcttaa gcagctgctc cgccgacatt |
| 601 | aaagacattt tgaacgaggc cggcaccatg caacttcttc agcagcagca acaacagcag |
| 661 | cagcaccaac agcagcacca acagcaccaa cagcagcagg aggtaatctc cgaaggcagc |
| 721 | agcgcaagag ccagggaggc cacgggggct ccctcttcct ccaaggatag ttacctaggg |
| 781 | ggcaattcaa ccatatctga cagtgccaag gagttgtgta aagcagtgtc tgtgtccatg |
| 841 | ggattgggtg tggaagcatt ggaacatctg agtccagggg aacagcttcg gggagactgc |
| 901 | atgtacgcgt cgctcctggg aggtccaccc gcggtgcgtc ccactccttg tgcgccgctg |
| 961 | cccgaatgca aaggtcttcc cctggacgaa ggcccaggca aaagcactga agagactgct |
| 1021 | gagtattcct ctttcaaggg aggttacgcc aaaggattgg aaggtgagag cttggggtgc |
| 1081 | tctggcagca gtgaagcagg tagctctggg acacttgaga tcccgtcctc tctgtctctg |
| 1141 | tataaatctg gagcactaga cgaggcagca gcataccaga atcgcgacta ctacaacttt |
| 1201 | ccgctggctc tgtccgggcc gccgcacccc ccgcccccta cccatccaca cgcccgtatc |
| 1261 | aagctggaga acccattgga ctacggcagc gcctgggctg cggcggcagc gcaatgccgc |
| 1321 | tatggggact tgggtagtct acatggaggg agtgtagccg ggcccagcac tggatcgccc |
| 1381 | ccagccacca cctcttcttc ctggcatact ctcttcacag ctgaagaagg ccaattatat |
| 1441 | gggccaggag gcgggggcgg cagcagcagc ccaagcgatg ccgggcctgt agccccctat |
| 1501 | ggctacactc ggccccctca ggggctgaca agccaggaga gtgactactc tgcctccgaa |
| 1561 | gtgtggtatc ctggtggagt tgtgaacaga gtaccctatc ccagtcccaa ttgtgtcaaa |
| 1621 | agtgaaatgg gaccttggat ggagaactac tccggacctt atggggacat gcgtttggac |
| 1681 | agtaccaggg accatgtttt acccatcgac tattactttc caccccagaa gacctgcctg |
| 1741 | atctgtggag atgaagcttc tggctgtcac tacggagctc tcacttgtgg cagctgcaag |
| 1801 | gtcttcttca aaagagccgc tgaagggaaa cagaagtatc tatgtgccag cagaaacgat |
| 1861 | tgtaccattg ataaatttcg gaggaaaaat tgcccatctt gtcgtctccg gaaatgttat |
| 1921 | gaagcaggga tgactctggg agctcgtaag ctgaagaaac ttggaaatct aaaactacag |
| 1981 | gaggaaggag aaaactccaa tgctggcagc cccactgagg acccatccca gaagatgact |
| 2041 | gtatcacaca ttgaaggcta tgaatgtcag cctatctttc ttaacgtcct ggaagccatt |
| 2101 | gagccaggag tggtgtgtgc cggacatgac aacaaccaac cagattcctt tgctgccttg |
| 2161 | ttatctagcc tcaatgagct tggagagagg cagcttgtgc atgtggtcaa gtgggccaag |
| 2221 | gccttgcctg gcttccgcaa cttgcatgtg gatgaccaga tggcggtcat tcagtattcc |
| 2281 | tggatgggac tgatggtatt tgccatgggt tggcggtcct tcactaatgt caactccagg |
| 2341 | atgctctact ttgcacctga cttggttttc aatgagtacc gcatgcacaa gtctcggatg |
| 2401 | tacagccagt gtgtgaggat gaggcacctg tctcaagagt ttggatggct ccaaataacc |
| 2461 | ccccaggaat tcctgtgcat gaaagcactg ctgctcttca gcattattcc agtggatggg |
| 2521 | ctgaaaaatc aaaaattctt tgatgaactt cgaatgaact acatcaagga actcgatcgc |
| 2581 | atcattgcat gcaaaagaaa gaatcccaca tcctgctcaa ggcgcttcta ccagctcacc |
| 2641 | aagctcctgg attctgtgca gcctattgca agagagctgc atcagttcac ttttgacctg |
| 2701 | ctaatcaagt cccatatggt gagcgtggac tttcctgaaa tgatggcaga gatcatctct |
| 2761 | gtgcaagtgc ccaagatcct ttctgggaaa gtcaagccca tctatttcca cacacagiga |
| 2821 | agatttggaa accctaatac ccaaaaccca ccttgttccc tttccagatg tcttctgcct |
| 2881 | gttatataac tctgcactac ttctctgcag tgccttgggg gaaattcctc tactgatgta |
| 2941 | cagtcagacg tgaacaggtt cctcagttct atttcctggg cttctcct |
| 6. Genbank Accession No. X59592. Mouse protein for androgen | |
| receptor. | |
| MEVQLGLGRVYPRPPSKTYRGAFQNLFQSVREAIQNPGPRHPEAANIAPPGACLQQRQETSPRRRRRQQ | |
| HTEDGSPQAHIRGPTGYLALEEEQQPSQQQAASEGHPESSCLPEPGAATAPGKGLPQQPPAPPDQDDSA | |
| APSTLSLLGPTFPGLSSCSADIKDILNEAGTMQLLQQQQQQQQHQQQHQQHQQQQEVISEGSSARAREA | |
| TGAPSSSKDSYLGGNSTISDSAKELCKAVSVSMGLGVEALEHLSPGEQLRGDCMYASLLGGPPAVRPTP | |
| CAPLPECKGLPLDEGPGKSTEETAEYSSFKGGYAKGLEGESLGCSGSSEAGSSGTLEIPSSLSLYKSGA | |
| LDEAAAYQNRDYYNFPLALSGPPHPPPPTHPHARIKLENPLDYGSAWAAAAAQCRYGDLGSLHGGSVAG | |
| PSTGSPPATTSSSWHTLFTAEEGQLYGPGGGGGSSSPSDAGPVAPYGYTRPPQGLTSQESDYSASEVWY | |
| PGGVVNRVPYPSPNCVKSEMGPWMENYSGPYGDMRLDSTRDHVLPIDYYFPPQKTCLICGDEASGCHYG | |
| ALTCGSCKVFFKRAAEGKQKYLCASRNDCTIDKFRRKNCPSCRLRKCYEAGMTLGARKLKKLGNLKLQE | |
| EGENSNAGSPTEDPSQKMTVSHIEGYECQPIFLNVLEAIEPGVVCAGHDNNQPDSFAALLSSLNELGER | |
| QLVHVVKWAKALPGFRNLHVDDQMAVIQYSWMGLMVFAMGWRSFTNVNSRMLYFAPDLVFNEYRMHKSR | |
| MYSQCVRMRHLSQEFGWLQITPQEFLCMKALLLFSIIPVDGLKNQKFFDELRMNYIKELDRIIACKRKN | |
| PTSCSRRFYQLTKLLDSVQPIARELHQFTFDLLIKSHMVSVDFPEMMAEIISVQVPKILSGKVKPIYFH | |
| TQ” | |
| 7. Genbank Accession No. X59592. Mouse mRNA for androgen | |
| receptor. | |
| 1 | gcttcccgca ggtgggcagc tagctgcaga tactacatca tcagtcagga gaactcttca |
| 61 | gagcaagaga cgaggaggca ggataaggga attcggtgga agctacagac aagctcaagg |
| 121 | atggaggtgc agttagggct gggaagggtc tacccacggc ccccalccaa gacctatcga |
| 181 | ggagcgttcc agaatctgtt ccagagcgtg cgcgaagcga tccagaaccc gggccccagg |
| 241 | caccctgagg ccgctaacat agcacctccc ggcgcctgtt tacagcagag gcaggagact |
| 301 | agcccccggc ggcggcggcg gcagcagcac actgaggatg gttctcctca agcccacatc |
| 361 | agaggcccca caggctacct ggccctggag gaggaacagc agccttcaca gcagcaggca |
| 421 | gcctccgagg gccaccctga gagcagctgc ctccccgagc ctggggcggc caccgctcct |
| 481 | ggcaaggggc tgccgcagca gccaccagct cctccagatc aggatgactc agctgcccca |
| 541 | tccacgttgt ccctgctggg ccccactttc ccaggcttaa gcagctgctc cgccgacatt |
| 601 | aaagacattt tgaacgaggc cggcaccatg caacttcttc agcagcagca acaacagcag |
| 661 | cagcaccaac agcagcacca acagcaccaa cagcagcagg aggtaatctc cgaaggcagc |
| 721 | agcgcaagag ccagggaggc cacgggggct ccctcttcct ccaaggatag ttacctaggg |
| 781 | ggcaattcaa ccatatctga caglgccaag gagttgtgta aagcagtgtc tgtgtccatg |
| 841 | ggattgggtg tggaagcatt ggaacatctg agtccagggg aacagcttcg gggagactgc |
| 901 | atgtacgcgt cgctcctggg aggtccaccc gcggtgcgtc ccactccttg tgcgccgctg |
| 961 | cccgaatgca aaggtcttcc cctggacgaa ggcccaggca aaagcactga agagactgct |
| 1021 | gagtattcct ctttcaaggg aggttacgcc aaaggattgg aaggtgagag cttggggtgc |
| 1081 | tctggcagca gtgaagcagg tagctctggg acacttgaga tcccgtcctc tctgtctctg |
| 1141 | tataaatctg gagcactaga cgaggcagca gcataccaga atcgcgacta ctacaacttt |
| 1201 | ccgctggctc tgtccgggcc gccgcacccc ccgcccccta cccatccaca cgcccgtatc |
| 1261 | aagctggaga acccattgga ctacggcagc gcctgggctg cggcggcagc gcaatgccgc |
| 1321 | tatggggact tgggtagtct acatggaggg agtgtagccg ggcccagcac tggatcgccc |
| 1381 | ccagccacca cctcttcttc ctggcatact ctcttcacag ctgaagaagg ccaattatat |
| 1441 | gggccaggag gcgggggcgg cagcagcagc ccaagcgatg ccgggcctgt agccccctat |
| 1501 | ggctacactc ggccccctca ggggctgaca agccaggaga gtgactactc tgcctccgaa |
| 1561 | gtgtggtatc ctggtggagt tgtgaacaga gtaccctatc ccagtcccaa ttgtgtcaaa |
| 1621 | agtgaaatgg gaccttggat ggagaactac tccggacctt atggggacat gcgtttggac |
| 1681 | agtaccaggg accatgtttt acccatcgac tattactttc caccccagaa gacctgcctg |
| 1741 | atctgtggag atgaagcttc tggctgtcac tacggagctc tcacttgtgg cagctgcaag |
| 1801 | gtcttcttca aaagagccgc tgaagggaaa cagaagtatc tatgtgccag cagaaacgat |
| 1861 | tgtaccattg ataaatttcg gaggaaaaat tgcccatctt gtcgtctccg gaaatgttat |
| 1921 | gaagcaggga tgactctggg agctcgtaag ctgaagaaac ttggaaatct aaaactacag |
| 1981 | gaggaaggag aaaactccaa tgctggcagc cccactgagg acccatccca gaagatgact |
| 2041 | gtatcacaca ttgaaggcta tgaatgtcag cctatctttc rtaacgtcct ggaagccatt |
| 2101 | gagccaggag tggtgtgtgc cggacatgac aacaaccaac cagattcctt tgctgccttg |
| 2161 | ttatctagcc tcaatgagct tggagagagg cagcttgtgc atgtggtcaa gtgggccaag |
| 2221 | gccttgcctg gcttccgcaa cttgcatgtg gatgaccaga tggcggtcat tcagtattcc |
| 2281 | tggatgggac tgatggtatt tgccatgggt tggcggtcct tcactaatgt caactccagg |
| 2341 | atgctctact ttgcacctga cttggttttc aatgagtacc gcatgcacaa gtctcggatg |
| 2401 | tacagccagt gtgtgaggat gaggcacctg tctcaagagt ttggatggct ccaaataacc |
| 2461 | ccccaggaat tcctgtgcat gaaagcactg ctgctcttca gcattattcc agtggatggg |
| 2521 | ctgaaaaatc aaaaattctt tgatgaactt cgaatgaact acatcaagga actcgatcgc |
| 2581 | atcattgcat gcaaaagaaa gaatcccaca tcctgctcaa ggcgcttcta ccagctcacc |
| 2641 | aagctcctgg attctgtgca gcctattgca agagagctgc atcagttcac ttttgacctg |
| 2701 | ctaatcaagt cccatatggt gagcgtggac tttcctgaaa tgatggcaga gatcatctct |
| 2761 | gtgcaagtgc ccaagatcct ttctgggaaa gtcaagccca tctatttcca cacacagtga |
| 2821 | agatttggaa accctaatac ccaaaaccca ccttgttccc tttccagatg tcttctgcct |
| 2881 | gttatataac tctgcactac ttctctgcag tgccttgggg gaaattcctc tactgatgta |
| 2941 | cagtcagacg tgaacaggtt cctcagttct atttcctggg cttctcct |
| 8. Genbank Accession No. M37890. Mouse androgen receptor protein | |
| MEVQLGLGRVYPRPPSKTYRGAFQNLFQSVREAIQNPGPRHPEAANIAPPGACLQQRQETSPRRRRR | |
| QQHTEDGSPQAHIRGPTGYLALEEEQQPSQQQAASEGHPESSCLPEPGAATAPGKGLPQQPPAPPDQDD | |
| SAAPSTLSLLGPTFPGLSSCSADIKDILNEAGTMQLLQQQQQQQQHQQQHQQHQQQQEVISEGSSARAR | |
| EATGAPSSSKDSYLGGNSTISDSAKELCKAVSVSMGLGVEALEHLSPGEQLRGDCMYASLLGGPPAVRP | |
| TPCAPLPECKGLPLDEGPGKSTEETAEYSSFKGGYAKGLEGESLGCSGSSEAGSSGTLEIPSSLSLYKS | |
| GALDEAAAYQNRDYYNFPLALSGPPHPPPPTHPHARIKLENPLDYGSAWAAAAAQCRYGDLGSLHGGSV | |
| AGPSTGSPPATTSSSWHTLFTAEEGQLYGPGGGGGSSSPSDAGPVAPYGYTRPPQGLTSQESDYSASEV | |
| WYPGGVVNRVPYPSPNGVKSEMGPWMENYSGPYGDMRLDSTRDHVLPIDYYFPPQKTCLICGDEASGCH | |
| YGALTCGSCKVFFKRAAEGKQKYLCASRNDCTIDKFRRKNCPSCRLRKCYEAGMTLGARKLKKLGNLKL | |
| QEEGENSNAGSPTEDPSQKMTVSHIEGYECQPIFLNVLEAIEPGVVCAGHDNNQPDSFAALLSSLNELG | |
| ERQLVHVVKWAKALPGFRNLHVDDQMAVIQYSWMGLMVFAMGWRSFTNVNSRMLYFAPDLVFNEYRMHK | |
| SRMYSQCVRMRHLSQEFGWLQITPQEFLCMKALLLFSIIPVDGLKNQKFFDELRMNYIKELDRIIACKR | |
| KNPTSCSRRFYQLTKLLDSVQPIARELHQFTFDLLIKSHMVSVDFPEMMAEIISVQVPKILSGKVKPIY | |
| FHTQ | |
| 9. Genbank Accession No. M37890. Mouse androgen receptor mRNA, | |
| complete cds | |
| 1 | atggaggtgc agttagggct gggaagggtc tacccacggc ccccatccaa gacctatcga |
| 61 | ggagcgttcc agaatctgtt ccagagcgtg cgcgaagcga tccagaaccc gggccccagg |
| 121 | caccctgagg ccgctaacat agcacctccc ggcgcctgtt tacagcagag gcaggagact |
| 181 | agcccccggc ggcggcggcg gcagcagcac actgaggatg gttctcctca agcccacatc |
| 241 | agaggcccca caggctacct ggccctggag gaggaacagc agccttcaca gcagcaggca |
| 301 | gcctccgagg gccaccctga gagcagctgc ctccccgagc ctggggcggc caccgctcct |
| 361 | ggcaaggggc tgccgcagca gccaccagct cctccagatc aggatgactc agctgcccca |
| 421 | tccacgttgt ccctgctggg ccccactttc ccaggcttaa gcagctgctc cgccgacatt |
| 481 | aaagacattt tgaacgaggc cggcaccatg caacttcttc agcagcagca acaacagcag |
| 541 | cagcaccaac agcagcacca acagcaccaa cagcagcagg aggtaatctc cgaaggcagc |
| 601 | agcgcaagag ccagggaggc cacgggggct ccctcttcct ccaaggatag ttacctaggg |
| 661 | ggcaattcaa ccatatctga cagtgccaag gagttgtgta aagcagtgtc tgtgtccatg |
| 721 | ggattgggtg tggaagcatt ggaacatctg agtccagggg aacagcttcg gggagactgc |
| 781 | atgtacgcgt cgctcctggg aggtccaccc gcggtgcgtc ccactccttg tgcgccgctg |
| 841 | cccgaatgca aaggtcttcc cctggacgaa ggcccaggca aaagcactga agagactgct |
| 901 | gagtattcct ctttcaaggg aggttacgcc aaaggattgg aaggtgagag cttggggtgc |
| 961 | tctggcagca gtgaagcagg tagctctggg acacttgaga tcccgtcctc tctgtctctg |
| 1021 | tataaatctg gagcactaga cgaggcagca gcataccaga atcgcgacta ctacaacttt |
| 1081 | ccgctggctc tgtccgggcc gccgcacccc ccgcccccta cccatccaca cgcccgtatc |
| 1141 | aagctggaga acccattgga ctacggcagc gcctgggctg cggcggcagc gcaatgccgc |
| 1201 | tatggggact tgggtagtct acatggaggg agtgtagccg ggcccagcac tggatcgccc |
| 1261 | ccagccacca cctcttcttc ctggcatact ctcttcacag ctgaagaagg ccaattatat |
| 1321 | gggccaggag gcgggggcgg cagcagcagc ccaagcgatg ccgggcctgt agccccctat |
| 1381 | ggctacactc ggccccctca ggggctgaca agccaggaga gtgactactc tgcctccgaa |
| 1441 | gtgtggtacc ctggtggagt tgtgaacaga gtaccctatc ccagtcccaa ttgtgtcaaa |
| 1501 | agtgaaatgg gaccttggat ggagaactac tccggacctt atggggacat gcgtttggac |
| 1561 | agtaccaggg accatgtttt acccatcgac tattactttc caccccagaa gacctgcctg |
| 1621 | atctgtggag atgaagcttc tggctgtcac tacggagctc tcacttgtgg cagctgcaag |
| 1681 | gtcttcttca aaagagccgc tgaagggaaa cagaagtatc tatgtgccag cagaaacgat |
| 1741 | tgtaccattg ataaatttcg gaggaaaaat tgcccatctt gtcgtctccg gaaatgttat |
| 1801 | gaagcaggga tgactctggg agctcgtaag ctgaagaaac ttggaaatct aaaactacag |
| 1861 | gaggaaggag aaaactccaa tgctggcagc cccactgagg acccatccca gaagatgact |
| 1921 | gtatcacaca ttgaaggcta tgaatgtcag cctatctttc ttaacgtcct ggaagccatt |
| 1981 | gagccaggag tggtgtgtgc cggacatgac aacaaccaac cagattcctt tgctgccttg |
| 2041 | ttatctagcc tcaatgagct tggagagagg cagcttgTgc atgtggtcaa gtgggccaag |
| 2101 | gccttgcctg gcttccgcaa cttgcatgtg gatgaccaga tggcggtcat tcagtattcc |
| 2161 | tggatgggac tgatggtatt tgccatgggt tggcggtcct tcactaatgt caactccagg |
| 2221 | atgctctact ttgcacctga cttggttttc aatgagtacc gcatgcacaa gtctcggatg |
| 2281 | tacagccagt gtgtgaggat gaggcacctg tctcaagagt ttggatggct ccaaataacc |
| 2341 | ccccaggaat tcctgtgcat gaaagcactg ctgctcttca gcattattcc agtggatggg |
| 2401 | ctgaaaaatc aaaaattctt tgatgaactt cgaatgaact acatcaagga actcgatcgc |
| 2461 | atcattgcat gcaaaagaaa gaatcccaca tcctgctcaa ggcgcttcta ccagctcacc |
| 2521 | aagctcctgg attctgtgca gcctattgca agagagctgc atcagttcac ttttgacctg |
| 2581 | ctaatcaagt cccatatggt gagcgtggac tttcctgaaa tgatggcaga gatcatctct |
| 2641 | gtgcaagtgc ccaagatcct ttctgggaaa gtcaagccca tctatttcca cacacagtga |

10. SEQ ID NO:10 Sequence flanking of mouse AR exon2: sequences of
exon 2 are underlined.
5′-CACCCCCCCAATCCCCTACCCACCCACTCCCCCTTTTTGGCCCTGGCGTTCCCCTGTACTGGGGCA
TATAAAGTTTGCAAGTCCAATGGGCCTCTCTCTTTGCCATGATGGCCGACTAGGCCATCTTTTGATACA
TATGCAGCTAAAGACAAGAGCTCCCGGGTACTGGTTAGTTCATATTGTTGTTCCACCTATAGGGTTGCA
GTTCCCTTTAGCTCCTTGGGTAATTTCTCTAGCTCCTCCATTAGGGGCCGTGTGACCCATCCAATAGCT
GACTGTGATCATCCACTTCTGTGTTTGCTAGGCCCCGACATAGTCTCACAAGAGAGAGCTATAACTGGG
TCCTTTCAGCGAAATCTTGCTAGTGTATGCAATGGTGTCAGCATTTGGAAGCTGATTATGGGATGGATC
CCTGCATATGGCATCTATTACATTTTTGTTACAGAACAGGGAAAGGGACACTGAGAGACTCAAGAAGAA
AGAAAAGGAATTAATACAAAAGAACAGTGAAAGCTGGTATGATAATACTAATTTATCCTTTACTTGTAT
ATTAATATCAAGAGTAACTCATACATCTGATTTATGTTGTCAGAGCAATAACTCAGTACTACTGGTAGC
AATATTGNTGTTTTTACAGGGTAAGACTCTAGGCTCCAAGAGCTAAAATATATAAAATTCTTCTGGTAT
TTGATAAGGCTGATCATAGGCCTCTCTCTGGAAGAAGTAAGATAGAGTTATGTTCATGCCATTTAATGA
CTGTATATGTCGTCATTAATGCATCACATTAAGTTGATACCTTAACCTCTGCTTAACTTCCTTCTCTTA
CAAATGCAGAGCTCATGAGATTGGCTATTCCCTCAGAACCTGTTTAATTCCTTGGCAGGATTCAAAGTG
TCCATAGGAAACCTTACAAACACTCTGTCCAGAGAAGGTCTCAAAAGAGTTCAGCTTTACACTGATTCA
CTCGAGCAATCCATAGAATAGTCACTTGGATGTATGTACAGTTTCTCAGAAGACCGTAGAATTCTGATC
GATGTCTGCCATCCACTGACATATGTTGCTTTGTTCTCTCTCTGTCTCTGTGTGTGTCTTTTTCAGTTT
GGACAGTACCAGGGACCATGTTTTACCCATCGACTATTACTTTCCACCCCAGAAGACCTGCCTGATCTG
TGGAGATGAAGCTTCTGGCTGTCACTACGGAGCTCTCACTTGTGGCAGCTGCAAGGTCTTCTTCAAAAG
AGCCGCTGAAGGTAAAAAGTCTTACCTACTTCCTGATATTTTCCCCTTCTCTTTTGCCTAGCAGAGAAT
GACAGTGACCTTCCAGGGCATTCTGATAATCCCAGAGACTGAGTCATTAGCAAGGGCCCTCTCACAGTA
CATGTAAGATCAAAGAAGCCCATGGTTATATTTGCTGAGCTGTCTTGGCTGCCCTGGTTGTACAAGCAA
TGATGGTGATGTAGGTGGTCCCAGCTGGTGCTTGGTGGCTCCCAGGACTGGAAGCAAAATTAATGATTT
GAAAAATTAAATTTCCTTCCTGCTTGTTTTCAACTCTGCTTCCTAGTGAGGAAAAGAAAACTTGTCCTT
ATTAGAGAGGTTAGAAGTGGAGAAACCCCAACTGAGTATACAGGCTGTTTTCTGTAGAGAATATGAGAC
TGTTCCTTAGCAAAAGCTTCCTGGCTTTAACCCCAGAAAAGGAAGTGTTCTCACTGTTCAGCAGACCAT
CAGTGTCTGCACCTGCTCCCTCCTGCTTGCTGCCTCTTTGGGACCTCTCTTTGCAATAAGGGACTCCAA
NGCANGAAAAAAACTCAGAGAGAAGCATCAGAGGACTGCTTTCAGGGCATGACAGTTGGTTCAAGAATC
CCAACGTAACTTGCATTTTGTATCCAGCTAAGTGGGATGGAGCCTTTACTTGTTATCTGCACTAATTAT
GATGTTTCTAACCTACATCATCTAGCAGAAACACCCACTCCAGGCCTTTACTGTAGTCTTAGTGATCCC
TCCCTTCTTAATCACAGGGTGGGGGTGGGAGCTTAAACCTTTATTCATACACTCTACTACCATCCCTCA
GTCTGGTACTCCTTTCTCAAAGAGTCACTGGAAAGCTGCCCCTACATGGTCTACTGTGGCTGCAGACTC
AGTTTTAAAGATTCCTTTGCAACTCTGCCCTGGTCTCTGGCTTCCCACCAAGGGGGANCTTCCGGCCAG
GGAGGTTTTCCTT-3′
11. SEQ ID NO: 11
CGGAATTCGTGGAAGCT
12. SEQ ID NO: 12
GAAAGATCTACATCAGTAG-AGG
13. SEQ ID NO: 13
GTCGGGCCCTATCCCAGTCCCACTTGCTCGAGCAAGTGGGACTGGGATAGGGCTTTTTTGAATT-CGC
14. SEQ ID NO: 14 Forward primer for Probasin
ATC ATC CTT CTG CTC ACA CTG CAT G
15. SEQ ID NO: 15 Reverse primer for Probasin
ACA GTT GTC CGT GTC CAT GAT ACG C
16. SEQ ID NO: 16 Forward primer for prostatic secretory protein-94
(PSP94)
CCT GTA AGG AGT CCT GCT TTG TC
17. SEQ ID NO: 17 Reverse primer for prostatic secretory protein-94
(PSP94)
ATG CTG GCT CTG CCT TCT GAG T
18. SEQ ID NO: 18 Forward primer for Nkx3.1
AGA CAC GCA CTG AAC CCG AGT CTG ATG CAC
19. SEQ ID NO: 19 Reverse primer for Nkx3.1
AGA CAG TAC AGG TAG GGG TAG TAG GGA TAG C
hAR siRNA
5-GTCGGGCCCTATCCCAGTCCCACTTGCTCGAGCAAGTGGG-3
5-GCGAATTCAAAAAGCCCTATCCCAGTCCCACTTGCTCGAG-3

| | |
|---|---|
| MAR exon-2-S | [symbol] ¦-GGACAGTACCAGGGACCATG- [symbol] ¦ |
| MARexon-2-AS | [symbol] ¦-TCCGTAGTGACAGCCAGAAG- [symbol] ¦ |
| M-TGF£]1-S | TAATGGTGGACCGCAACAAC |
| M-TGF£]1-AS | GTATTCCGTCTCCTTGGTTCAG |
| M-TGF£]2-S | CAGAGCGGAGGGTGAATG |
| M-TGF£]2-AS | GGCGAAGGCAGCAATTATC |
| M-TGF£]3-S | AGGAGTGGACAATGAAGATG |
| M-TGF£]3-AS | TGAGCAGAAGTTGGCATAG |
| mSmad4-F | CACTATGAGCGGGTTGTC |
| mSmad4-R | GGTGCTGGTGGCGTTAGA |
| mSmad2-F | CTACACCCACTCCATTCC |
| mSmad2-R | GCAGGTTCCGAGTAAGTAA |
| mSmad3-F | GGGCTTTGAGGCTGTCTA |
| mSmad3-R | AAGGGTCCATTCAGGTGTA |
| mCTGF-S | [symbol] ¦-ATCTCCACCCGAGTTACCA- [symbol] ¦ |
| mCTGF-AS | [symbol] ¦-AACTTAGCCCTGTATGTGTTCA- [symbol] ¦ |
| hCTGF-S | [symbol] ¦-AATGCTGCGAGGAGTGGG- [symbol] ¦ |
| hCTGF-AS | [symbol] ¦-GGCTCTAATCATAGTTGGGTCT- [symbol] ¦ |
| mEGF-S | [symbol] ¦-TGGTCCTGCTGCTCCTCTTG- [symbol] ¦ |
| mEGF-AS | [symbol] ¦-CCGCTGCTGCTCACACTTC- [symbol] ¦ |
| hEGF-S | [symbol] ¦-TACCGAGACCTGAAGTGG- [symbol] ¦ |
| hEGF-AS | [symbol] ¦-TCTGAGTCCTGTAGTAGTGGG- [symbol] ¦ |
| mHGF-S | [symbol] ¦-AGAGGTACGCTACGAAGTC- [symbol] ¦ |
| mHGF-AS | [symbol] ¦-GCTTGCCATCAGGATTGC- [symbol] ¦ |
| h-HGF | AGGGGCACTGTCAATACCATT |
| | CGTGAGGATACTGAGAATCCCAA |
| mIGF1-S | [symbol] ¦-GGTGGATGCTCTTCAGTTC- [symbol] ¦ |
| mIGF1-AS | [symbol] ¦-TTTGTAGGCTTCAGTGGG- [symbol] ¦ |
| hIGF1-S | [symbol] ¦-TATTTCAACAAGCCCACAG- [symbol] ¦ |
| hIGF1-AS | [symbol] ¦-ATACATCTCCAGCCTCCTTA- [symbol] ¦ |
| Mouse VEGF | F: [symbol] ¦-gga aca ccg aca aac cca- [symbol] ¦ |
| | R: [symbol] ¦-tcc cca aag cac agc aat- [symbol] ¦ |
| mFGF2-S | [symbol] ¦-AACGGCGGCTTCTTCCTG- [symbol] ¦ |
| mFGF2-AS | [symbol] ¦-TGGCACACACTCCCTTGATAG- [symbol] ¦ |
| mFGF7-S | [symbol] ¦-TCCTGCCAACTCTGCTCTAC- [symbol] ¦ |
| mFGF7-AS | [symbol] ¦-CTTTCACTTTGCCTCGTTTGTC- [symbol] ¦ |
| mFGF10-S | [symbol] ¦-CTGCTGTTGCTGCTTCTTG- [symbol] ¦ |
| mFGF10-AS | [symbol] ¦-TGACCTTGCCGTTCTTCTC- [symbol] ¦ |
| SDF1-S | [symbol] ¦CTGTGCCCTTCAGATTGTT [symbol] ¦ |
| SDF1-AS | [symbol] ¦GGCGGAGTGTCTTTATGC [symbol] ¦ |
| beta-actin-S | TGTGCCCATCTACGAGGGGTATGC |
| beta-actin-AS | GGTACATGGTGCCGCCAGACA |

What is claimed is:

1. A method of inhibiting cellular proliferation in a subject comprising administering to the subject a vector comprising an androgen or androgen receptor gene operably linked to an epithelial tissue specific promoter.

2. A method of treating a cancer in a subject comprising administering to the subject a vector comprising an androgen or androgen receptor gene operably linked to an epithelial tissue specific promoter.

3. The method of claim 2, wherein the cancer is prostate cancer.

4. The method of claim 2, further comprising administering to the subject an anti-androgen or anti-androgen receptor agent, wherein the agent is targeted to the stromal tissue.

5. A method of treating a cancer in a subject comprising administering to the subject an anti-androgen or anti-androgen receptor agent, wherein the agent is targeted to the stromal tissue.

6. The method of any of claim 2, wherein the cancer is prostate cancer.

7. The method of claim 4, wherein the agent is an anti-androgen or anti-androgen receptor antibody.

8. The method of claim 7, wherein the agent is an antibody fusion protein comprising a stromal tissue targeting site.

9. The method of claim 5, wherein the agent is an anti-androgen or anti-androgen receptor siRNA.

10. The method of claim 9, wherein the siRNA comprises the sequence set forth in SEQ ID NO: 13, 20 or 21.

11. The method of claim 5, wherein the agent is ASC-J9.

12. A vector comprising an androgen receptor gene operably linked to an androgen receptor promoter.

13. (canceled)

14. (canceled)

15. A method of treating a cancer or cellular proliferation comprising administering to a subject the vector of claim 12.

16. (canceled)

17. A vector comprising an androgen gene operably linked to an androgen promoter.

18. (canceled)

19. A method of inhibiting cellular proliferation or cancer comprising administering to a subject the vector of claim 17.

20. (canceled)

21. (canceled)

22. A vector comprising an androgen or androgen receptor gene operably linked to epithelial tissue specific promoter.

23. A method of inhibiting cellular proliferation or cancer comprising administering to a subject the vector of claim 22.

24. (canceled)

25. A method of screening for an agent that inhibits prostate growth comprising administering the agent to a prostate cell and monitoring the level of epithelial androgen receptor on the cell, wherein an increase in epithelial androgen receptor relative to a control indicates an agent that inhibits prostate growth.

26. A method of screening for an agent that inhibits androgen dependent tumor growth comprising administering the agent to a prostate cell and monitoring the level of epithelial androgen receptor on the cell, wherein an increase in epithelial androgen receptor relative to a control indicates an agent that inhibits androgen dependent tumor growth.

27. A method of screening for an agent that inhibits prostate growth comprising obtaining a tissue sample from a subject, administering the agent to the sample and monitoring the level of epithelial androgen receptor in the tissue, wherein an increase in epithelial androgen receptor relative to a control indicates an agent that inhibits prostate growth.

28. A method of screening for an agent that inhibits prostate growth comprising administering the agent to a prostate cell and monitoring the level of stromal androgen receptor on the cell, wherein an decrease in stromal androgen receptor relative to a control indicates an agent that inhibits prostate growth.

29. A method of screening for an agent that inhibits androgen dependent tumor growth comprising administering the agent to a prostate cell and monitoring the level of stromal androgen receptor on the cell, wherein an decrease in stromal androgen receptor relative to a control indicates an agent that inhibits androgen dependent tumor growth.

30. A method of screening for an agent that inhibits prostate growth comprising obtaining a tissue sample from a subject, administering the agent to the sample and monitoring the level of stromal androgen receptor in the tissue, wherein an decrease in stromal androgen receptor relative to a control indicates an agent that inhibits prostate growth.

31. A cell, wherein the cell has a disrupted AR gene, wherein the disrupted gene is produced by action of a recombinase operably linked to a promoter.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. A transgenic mammal comprising the cell of claims 31.

56. (canceled)

57. (canceled)

58. A transgenic mammal comprising a disrupted AR gene, wherein the disrupted gene is produced by action of a recombinase operably linked to a promoter.

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. A cell, wherein the cell is an Androgen Receptor (AR)-negative prostate metastatic cell, and wherein the cell is stably transfected with an AR gene under the control of an AR promoter.

83. (canceled)

84. (canceled)

85. (canceled)
